(19) United States
(12) Patent Application Publication
(10) Pub. No.: US 2005/0203086 A1
(43) Pub. Date: Sep. 15, 2005

(54) Methods of treatment using an EP2 selective receptor agonist

(75) Inventors: Alexander A. Constan, Waterford, CT (US); Prakash Keshary, Mansfield, CT (US); David B. MacLean, Middletown, RI (US); Vishwas M. Paralkar, Madison, CT (US); Doina Roman, Ann Arbor, MI (US); David D. Thompson, Gales Ferry, CT (US); Timothy M. Wright, Swampscott, MA (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/793,530

(22) Filed: Mar. 4, 2004

(51) Int. Cl. A61K 31/541; A61K 31/5377; A61K 31/496; A61K 31/454; A61K 31/433; A61K 31/4245

(52) U.S. Cl. 514/227.5; 514/362; 514/364; 514/381; 514/602; 514/235.2; 514/254.03; 514/326

(57) ABSTRACT

The present invention relates to methods of treating pulmonary hypertension, facilitating joint fusion, facilitating tendon and ligament repair, reducing the occurrence of secondary fracture, treating avascular necrosis, facilitating cartilage repair, facilitating bone healing after limb transplantation, facilitating liver regeneration, facilitating wound healing, reducing the occurrence of gastric ulceration, treating hypertension, facilitating the growth of tooth enamel or finger or toe nails, treating glaucoma, treating ocular hypertension, and repairing damage caused by metastatic bone disease using an EP₂ selective receptor agonist.

### FIELD OF THE INVENTION

The present invention relates to methods of treating pulmonary hypertension, facilitating joint fusion, facilitating tendon and ligament repair, reducing the occurrence of secondary fracture, treating avascular necrosis, facilitating cartilage repair, facilitating bone healing after limb transplantation, facilitating liver regeneration, facilitating wound healing, reducing the occurrence of gastric ulceration, treating hypertension, facilitating the growth of tooth enamel or finger or toe nails, treating glaucoma, treating ocular hypertension, and repairing damage caused by metastatic bone disease using an EP₂ selective receptor agonist.

### BACKGROUND OF THE INVENTION

Compounds that are prostaglandin receptor ligands are known to be useful to treat various diseases such as osteoporosis. A variety of natural prostaglandins such as PGE, PGD and PDF are associated with skeletal metabolism. PGE2 has been reported to stimulate bone formation, increase bone mass and bone strength in animal models of osteoporosis when administered systemically or locally. However, there are severe side effects associated with PGE2 such as diarrhea, gastrointestinal bleeding, decreased food consumption, dehydration, weight loss and decreased physical activity. Accordingly, PGE2 has not found widespread use in humans because of these side effects. Recently, four different subtypes of PGE2 receptors have been cloned. The four subtypes have been named EP₁, EP₂, EP₃ and EP₄, and research to better understand the pharmacology of the receptor subtypes is presently being conducted.

The present invention provides methods of treating pulmonary hypertension, facilitating joint fusion, facilitating tendon and ligament repair, reducing the occurrence of secondary fracture, treating avascular necrosis, facilitating cartilage repair, facilitating bone healing after limb transplantation, facilitating liver regeneration, facilitating wound healing, reducing the occurrence of gastric ulceration, treating hypertension, facilitating the growth of tooth enamel or finger or toe nails, treating glaucoma, treating ocular hypertension, and repairing damage caused by metastatic bone disease using an EP₂ selective receptor agonist. Certain EP₂ selective receptor agonists are known in the art. See, for example, U.S. Pat. No. 6,498,172.

### SUMMARY OF THE INVENTION

The present invention provides methods of treating pulmonary hypertension, facilitating joint fusion, facilitating tendon and ligament repair, reducing the occurrence of secondary fracture, treating avascular necrosis, facilitating cartilage repair, facilitating bone healing after limb transplantation, facilitating liver regeneration, facilitating wound healing, reducing the occurrence of gastric ulceration, treating hypertension, facilitating the growth of tooth enamel or finger or toe nails, treating glaucoma, treating ocular hypertension, and repairing damage caused by metastatic bone disease, the methods comprising administering to a patient in need thereof a therapeutically effective amount of an EP₂ selective receptor agonist.

The present invention also provides such methods wherein the EP₂ selective receptor agonist is a compound of Formula I 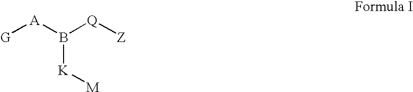
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
Bis N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkyleries each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-Q₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³,R R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
   a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
   b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

The present invention also provides methods of treating pulmonary hypertension, facilitating joint fusion, facilitating tendon and ligament repair, reducing the occurrence of secondary fracture, treating avascular necrosis, facilitating cartilage repair, facilitating bone healing after limb transplantation, facilitating liver regeneration, facilitating wound healing, reducing the occurrence of gastric ulceration, treating hypertension, facilitating the growth of tooth enamel or finger or toe nails, treating glaucoma, treating ocular hypertension, and repairing damage caused by metastatic bone disease, the methods comprising administering to a patient in need thereof a therapeutically effective amount of (3-(((4-tert-butyl-benzyl)-(pyridine-3-sulfonyl)-amino)-methyl)-phenoxy)-acetic acid or a pharmaceutically acceptable salt thereof.

### DETAILED DESCRIPTION OF THE INVENTION

Examples of EP₂ selective receptor agonists are set forth in U.S. Pat. No. 6,498,172. A preferred EP₂ selective receptor agonist that can be used in the present methods is a compound of Formula I as defined above.

A preferred group of compounds designated the A Group, comprises those compounds having the Formula I as shown above, prodrugs thereof and pharmaceutically acceptable salts of said compounds and said prodrugs, wherein B is N; Z is carboxyl, (C₁-C₆)alkoxycarbonyl or tetrazolyl; Ar is phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, 2H-pyrrolyl, 3H-pyrrolyl, pyrrolyl, 2-pyrrolinyl, 3-pyrrolinyl, pyrrolidinyl, 1,3-dioxolanyl, 2H-imidazolyl, 2-imidazolinyl, imidazolidinyl, 2-pyrazolinyl, pyrazolidinyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 2H-pyranyl, 4H-pyranyl, pyridyl, piperidinyl, 1,4-dioxanyl, morpholinyl, 1,4-dithianyl, thiomorpholinyl, piperazinyl, 1,3,5-triazinyl, 1,2,4-triazinyl, azepinyl, oxepinyl, thiepinyl, cyclopentenyl, cyclohexenyl, benzo(b)thienyl, benzoxazolyl, benzimidazolyl, benzthiazolyl, quinolinyl, isoquinolinyl, phthalazinyl, quinazolinyl, quinoxalinyl, naphthyl, tetralinyl, decalinyl, 2H-, 1-benzopyranyl and 1,4-benzodioxan; Ar¹, Ar², Ar³, Ar⁴ and Ar⁵ are each independently cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, 2H-pyrrolyl, 3H-pyrrolyl, pyrrolyl, 2-pyrrolinyl, 3-pyrrolinyl, pyrrolidinyl, 1,3-dioxolanyl, 2H-imidazolyl, 2-imidazolinyl, imidazolidinyl, 2-pyrazolinyl, pyrazolidinyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 2H-pyranyl, 4H-pyranyl, pyridyl, piperidinyl, 1,4-dioxanyl, morpholinyl, 1,4-dithianyl, thiomorpholinylpiperazinyl, 1,3,5-triazinyl, 1,2,4-triazinyl, azepinyl, oxepinyl, thiepinyl, 1,2,4-diazepinyl, cyclopentenyl, cyclohexenyl, cycloheptenyl, cyclooctenyl, cyclooctadienyl, indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, 1,4-benzodioxan, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl; and X is tetrahydrofuranyl, phenyl, thiazolyl, thienyl, pyridyl, pyrrazolyl, furanyl or pyrimidyl, wherein X is optionally mono-, di- or tri-substituted independently with chloro, fluoro, methoxy, difluoromethoxy, trifluoromethoxy, trifluoromethyl or methyl; and wherein each of said Ar, Ar¹ and Ar² groups are optionally substituted on carbon or nitrogen with up to three substituents independently selected from R³, R⁴ and R⁵; each of said Ar, Ar¹ and Ar² groups are optionally substituted independently on carbon or sulfur with one or two oxo groups; each of said Ar³, Ar⁴ and Ar⁵ groups are optionally substituted on carbon or nitrogen independently with up to three R³¹, R⁴¹ and R⁵¹ groups and each of said Ar³, Ar⁴ and Ar⁵ groups are optionally substituted independently on carbon or sulfur with one or two oxo groups.

A group of compounds within the A Group, designated the B Group, comprises those compounds, prodrugs thereof and pharmaceutically acceptable salts of said compounds and said prodrugs, wherein A is CO; G is oxy(C₁-C₆)alkylene; Q is
—(C₂-C₆)alkylene-O—(C₁-C₃)alkylene-,
—(C₄-C₈)alkylene-, said —(P₄—C₈)alkylene- optionally substituted with up to four substituents independently selected from fluoro or (C₁-C₄)alkyl,
—X—(C₂-C₅)alkylene-,
—(C₁-C₅)alkylene-X—,
—(C₁-C₃)alkylene-X—(C₁-C₃)alkylene-,
—(C₂-C₄)alkylene-O—X—(C₀-C₃)alkylene-, or
—(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl.

Another group of compounds which is preferred within the A Group, designated the C Group, comprises those compounds, prodrugs thereof and pharmaceutically acceptable salts of said compounds and said prodrugs, wherein A is CO; G is Ar; Q is
—(C₂-C₆)alkylene-O—(C₁-C₃)alkylene-,
—(C₄-C₈)alkylene-, said —(C₄-C₈)alkylene- optionally substituted with up to four substituents independently selected from fluoro or (C₁-C₄)alkyl,
—X—(C₂-C₅)alkylene-,
—(C₁-C₅)alkylene-X—,
—(C₁-C₃)alkylene-X—(C₁-C₃)alkylene-,
—(C₂-C₄)alkylene-O—X—(C₀-C₃)alkylene-, or
—(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl.

Another group of compounds which is preferred within the A Group, designated the D Group, comprises those compounds, prodrugs thereof and pharmaceutically acceptable salts of said compounds and said prodrugs, wherein A is CO; G is R¹R²-amino or amino substituted with Ar, or amino substituted with Ar(C₁-C₄)alkylene and R¹¹, wherein R¹¹ is H; Q is
—(C₂-C₆)alkylene-O—(C₁-C₃)alkylene-,
—(C₄-C₈)alkylene-, said —(C₄-C₈)alkylene- optionally substituted with up to four substituents independently selected from fluoro or (C₁-C₄)alkyl,
—X—(C₂-C₅)alkylene-,
—(C₁-C₅)alkylene-X—,
—(C₁-C₃)alkylene-X-(C₁-C₃)alkylene-,
—(C₂-C₄)alkylene-O—X—(C₀-C₃)alkylene-, or
—(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl; and
wherein R¹ and R² may be taken separately and are independently selected from H and (C₁-C₈)alkyl, or R¹ and R² are taken together to form a five- or six-membered azacycloalkyl, said azacycloalkyl optionally containing an oxygen atom.

Another group of compounds which is preferred within the G Group, designated the E Group, comprises those compounds, prodrugs thereof and pharmaceutically acceptable salts of said compounds and said prodrugs, wherein A is SO₂; G is R¹R²-amino, or amino substituted with Ar and R¹¹; Q is
—(C₂-C₆)alkylene-O—(C₁-C₃)alkylene-,
—(C₄-C₈)alkylene-, said —(C₄-C₈)alkylene- optionally substituted with up to four substituents independently selected from fluoro or (C₁-C₄)alkyl,
—X—(C₂-C₅)alkylene-,
—(C₁-C₅)alkylene-X—,
—(C₁-C₃)alkylene-X—(C₁-C₃)alkylene-,
—(C₂-C₄)alkylene-O—X—(C₀-C₃)alkylene-, or
—(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl; and
wherein R¹ and R² may be taken separately and are independently selected from H and (C₁-C₈)alkyl, or R¹ and R² are taken together to form a five- or six-membered azacycloalkyl, said azacycloalkyl optionally containing an oxygen atom.

Another group of compounds which is preferred within the A Group, designated the F Group, comprises those compounds, prodrugs thereof and pharmaceutically acceptable salts of said compounds and said prodrugs, wherein A is SO₂; G is Ar, Ar(C₁-C₂)alkylene or Ar¹—V—Ar²; Q is
—(C₂-C₆)alkylene-O—(C₁-C₃)alkylene-,
—(C₄-C₈)alkylene-, said —(C₄-C₈)alkylene- optionally substituted with up to four substituents independently selected from fluoro or (C₁-C₄)alkyl,
—X—(C₂-C₅)alkylene-,
—(C₁-C₅)alkylene-X—,
—(C₁-C₃)alkylene-X—(C₁-C₃)alkylene-,
—(C₂-C₄)alkylene-O—X—(C₀-C₃)alkylene-, or
—(C₀-C₄)alkylene-X—O—(C₁-C₃)alkylene-; and X is phenyl, pyrimidyl, pyridyl, thienyl, tetrahydrofuranyl, furanyl or thiazolyl, wherein X is optionally mono-, di- or tri-substituted with chloro, fluoro, methoxy, difluoromethoxy, trifluoromethoxy, trifluoromethyl or methyl.

A particularly preferred group of compounds within the F Group, designated the FA Group, comprises those compounds, prodrugs thereof and pharmaceutically acceptable salts of said compounds and said prodrugs, wherein G is Ar or Ar—(C₁-C₂)-alkylene; Ar is phenyl, furyl, thienyl, pyrrolyl, oxazolyl, thiazolyl, imidazolyl, pyrazolyl, isoxazolyl, pyridyl, pyridazinyl, pyrimidinyl, pyrazinyl, isothiazolyl, 1,2,3-triazolyl, 1,2,4-triazolyl or 1,3,4-thiadiazolyl wherein each of said Ar groups is optionally substituted on carbon or nitrogen with R¹, R² or R³; Ar⁴ is cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, pyrrolidinyl, 1,2,3-triazolyl, 1,2,4-triazolyl, pyranyl, thiomorpholinyl, piperazinyl, 1,3,5-triazinyl, 1,2,4-triazinyl, 1,2,3-triazinyl, azepinyl, oxepinyl or thiepinyl wherein each of said Ar⁴ groups is optionally mono- di- or tri-substituted on carbon or nitrogen with R³¹, R⁴¹ or R⁵¹; Ar⁵ is cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, pyrrolidinyl, 1,2,3-triazolyl, 1,2,4-triazolyl, pyranyl, 1,4-dioxanyl, thiomorpholinyl, piperazinyl, 1,3,5-triazinyl, 1,2,4-triazinyl, 1,2,3-triazinyl, azepinyl, oxepinyl or thiepinyl wherein each of said Ar⁵ groups is optionally mono- di- or tri-substituted on carbon or nitrogen with R³¹, R⁴¹ or R⁵¹; Q is —(C₅-C₇)-alkylene-, —(C₁-C₂)-alkylene-X—(C₁-C₂)-alkylene-, —(C₁-C₂)—X—O—(C₁-C₂)-alkylene-, —(C₂-C₄)-alkylene-thienyl-, —(C₂-C₄)-alkylene-furanyl- or —(C₂-C₄)-alkylene-thiazolyl-; X is phenyl, pyridyl, pyrimidyl or thienyl; and said X groups are optionally mono-, di- or tri-substituted with chloro, fluoro, methoxy, difluoromethoxy, trifluoromethoxy, trifluoromethyl or methyl; said —(C₂-C₄)-alkylene-furanyl- and —(C₂-C₄)-alkylene-thienyl- having a 2,5-substitution pattern, e.g.,

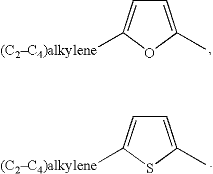

A preferred group of compounds within the FA Group, designated the FB Group, comprises those compounds, prodrugs thereof and pharmaceutically acceptable salts of said compounds and said prodrugs, wherein K is methylene, M is Ar⁴—Ar⁵, Ar⁴—O—Ar⁵ or Ar⁴—S—Ar⁵ and Ar is phenyl, pyridyl, pyrazolyl, imidazolyl, pyrimidyl, thienyl or thiazolyl, wherein Ar is optionally mono-, di- or tri-substituted on carbon or nitrogen with R³, R⁴ or R⁵.

A preferred group of compounds within the FB Group, designated the FC Group, comprises those compounds, prodrugs thereof and pharmaceutically acceptable salts of said compounds and said prodrugs, wherein M is Ar⁴—Ar⁵; Ar is phenyl, pyridyl or imidazolyl; Ar⁴ is phenyl, furanyl or pyridyl; and Ar⁵ is cyclopentyl, cyclohexyl, cycloheptyl, phenyl, pyridyl, imidazolyl, pyrimidyl, thienyl, pyridazinyl, pyrazinyl, imidazolyl, pyrazolyl or thiazolyl, wherein Ar, Ar⁴ and Ar⁵ are optionally mono, -di- or tri-substituted on carbon or nitrogen independently with chloro, fluoro, methyl, methoxy, difluoromethoxy, trifluoromethyl or trifluoromethoxy.

An especially preferred group of compounds within the FC Group, designated the FD Group, comprises those compounds, prodrugs thereof and pharmaceutically acceptable salts of said compounds and said prodrugs, wherein Q is —(C₅-C₇)alkylene-.

Another especially preferred group of compounds within the FC Group, designated the FE Group, comprises those compounds, prodrugs thereof and pharmaceutically acceptable salts of said compounds and said prodrugs, wherein Q is CH₂—X—CH₂— and X is metaphenylene optionally mono- or di-substituted with chloro, fluoro, methoxy, difluoromethoxy, trifluoromethoxy, trifluoromethyl or methyl.

A preferred group of compounds within the FE Group are those compounds, and pharmaceutically acceptable salts and prodrugs thereof, selected from (3-(((pyridine-3-sulfonyl)-(4-pyrimidin-5-yl-benzyl)-amino)-methyl)-phenyl)-acetic acid; (3-(((5-phenyl-furan-2-ylmethyl)-(pyridine-3-sulfonyl)-amino)-methyl)-phenyl)-acetic acid; (37(((pyridine-3-sulfonyl)-(4-pyrimidin-2-yl-benzyl)-amino)-methyl)-phenyl)-acetic acid; (3-(((pyridine-3-sulfonyl)-(4-thiazol-2-yl-benzyl)-amino)-methyl)-phenyl)-acetic acid; and (3-(((4-pyrazin-2-yl-benzyl)-(pyridine-3-sulfonyl)-amino)-methyl)-phenyl)-acetic acid.

An especially preferred compound within the FE Group is the compound wherein Ar is pyrid-3-yl; Z is carboxy; M is Ar⁴—Ar⁵ wherein Ar⁴ is a furanyl ring and Ar⁵ is phenyl wherein said phenyl moiety is substituted at the 5-position of said furanyl ring; and Q is —CH₂—X—CH₂— wherein X is metaphenylene.

Another especially preferred compound within the FE Group is the compound wherein Ar is pyrid-3-yl; Z is carboxy; M is Ar⁴—Ar⁵ wherein Ar⁴ is phenyl and Ar⁵ is pyrimid-2-yl and said pyrimid-2-yl moiety is substituted at the 4-position of said phenyl ring; and Q is —CH₂—X—CH₂— wherein X is metaphenylene.

Yet another especially preferred compound within th FE Group is the compound wherein Ar is pyrid-3-yl; Z is carboxy; M is Ar⁴—Ar⁵ wherein Ar⁴ is phenyl and Ar⁵ is thiazol-2-yl and said thiazol-2-yl moiety is substituted at the 4-position of said phenyl ring; and Q is —CH₂—X—CH₂— wherein X is metaphenylene.

Yet another especially preferred compound within the FE Group is the compound wherein Ar is pyrid-3-yl; Z is carboxy; M is Ar⁴—Ar⁵ wherein Ar⁴ is phenyl and Ar⁵ is pyrimid-5-yl and said pyrimid-5-yl moiety is substituted at the 4-position of said phenyl ring; and Q is —CH₂—X—CH₂— wherein X is metaphenylene.

Yet another especially preferred compound within the FE Group is the compound wherein Ar is pyrid-3-yl; Z is carboxy; M is Ar⁴—Ar⁵ wherein Ar⁴ is phenyl and Ar⁵ is pyrazin-2-yl and said pyrazin-2-yl is substituted at the 4-position of said phenyl ring; and Q is —CH₂—X—CH₂— wherein X is metaphenylene.

A preferred group of compounds within the FC Group, designated the G Group, comprises those compounds, prodrugs thereof and pharmaceutically acceptable salts of said compounds and said prodrugs, wherein Q is —(C₂-C₄)-alkylene-thienyl-, —(C₂-C₄)-alkylene-furanyl- or —(C₂-C₄)-alkylene-thiazolyl-.

An especially preferred compound within the G Group is 5-(3-((pyridine-3-sulfonyl)-(4-thiazol-2-yl-benzyl)-amino)-propyl)-thiophene-2-carboxylic acid.

An especially preferred compound within the G Group is the compound, prodrugs thereof and pharmaceutically acceptable salts of said compounds and said prodrugs, wherein Q is n-propylenyl; X is thienyl; Z is carboxy; Ar is 3-pyridyl; Ar⁴ is phenyl; and Ar⁵ is 2-thiazolyl; said 2-thiazolyl being substituted at the 4-position of said phenyl.

Another especially preferred group of compounds within the FC Group, designated the H Group, comprises those compounds, prodrugs thereof and pharmaceutically acceptable salts of said compounds and said prodrugs, wherein Q is —CH₂—X—O—CH₂—; Ar⁴ is phenyl or pyridyl; said phenyl and pyridyl are optionally substituted with chloro, fluoro, methoxy, difluoromethoxy, trifluoromethoxy, trifluoromethyl and methyl; and X is metaphenylene.

A preferred group of compounds within the H Group are (3-(((4-cyclohexyl-benzyl)-(pyridine-3-sulfonyl)-amino)-methyl)-phenoxy)-acetic acid; (3-(((pyridine-3-sulfonyl)-(4-pyridin-2-yl-benzyl)-amino)-methyl)-phenoxy)-acetic acid; (3-(((pyridine-3-sulfonyl)-(4-pyridin-3-yl-benzyl)-amino)-methyl)-phenoxy)-acetic acid; (3-(((pyridine-3-sulfonyl)-(4-pyridin-4-yl-benzyl)-amino)-methyl)-phenoxy)-acetic acid; and (3-(((pyridine-3-sulfonyl)-(4-thiazol-2-yl-benzyl)-amino)-methyl)-phenoxy)-acetic acid.

An especially preferred compound within the H Group is the compound, prodrugs thereof and pharmaceutically acceptable salts of said compounds and said prodrugs, wherein Ar is pyrid-3-yl; Z is carboxy; Ar⁴ is phenyl; Ar⁵ is cyclohexyl; and said cyclohexyl moiety is substituted at the 4-position of said phenyl ring.

Another especially preferred compound within the H Group is the compound wherein Ar is pyrid-3-yl; Z is carboxy; Ar⁴ is phenyl; Ar⁵ is thiazol-2-yl; and said thiazol-2-yl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 2-pyridyl; and said 2-pyridyl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 3-pyridyl; and said 3-pyridyl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 4-pyridyl; and said 4-pyridyl moiety is substituted at the 4-position of said phenyl ring.

A preferred group of compounds within the FA Group, designated the I Group, comprises those compounds, prodrugs thereof and pharmaceutically acceptable salts of said compounds and said prodrugs, wherein K is methylene, G is Ar; Ar is phenyl, pyridazinyl, pyrazolyl, pyrazinyl, pyridyl, imidazolyl, pyrimidyl, thienyl or thiazolyl, Ar is optionally mono-, di- or tri-substituted with R³, R⁴ or R⁵, and M is Ar³, wherein said Ar³ is cyclopentyl, cyclohexyl, phenyl, thienyl, pyridazinyl, pyrimidinyl, pyrazinyl, indolyl, benzofuryl, benzo(b)thienyl, benzoxazolyl, benzthiazolyl, quinolinyl, isoquinolinyl, naphthyl, tetralinyl, 2H-1-benzopyranyl or 1,4-benzodioxan and is optionally mono-, di- or tri-substituted with R³¹, chloro, fluoro, methyl, methoxy, difluoromethoxy, trifluoromethyl or trifluoromethoxy.

An especially preferred group of compounds within the I Group are (3-(((2,3-dihydro-benzo[1,4]dioxin-6-ylmethyl)-(pyridine-3-sulfonyl)-amino)-methyl)-phenyl)-acetic acid; and (3-((benzofuran-2-ylmethyl-(pyridine-3-sulfonyl)-amino)-methyl)-phenyl)-acetic acid.

An especially preferred compound within the I Group is the compound, prodrugs thereof and pharmaceutically acceptable salts of said compound and prodrugs, wherein Ar is pyrid-3-yl; Z is carboxy; M is 6-(1,4-benzodioxan); and Q is —CH₂—X—CH₂— wherein X is metaphenylene.

Another especially preferred compound within the I Group is the compound wherein Ar is pyrid-3-yl; Z is carboxy; M is 2-benzofuryl; and Q is —CH₂—X—CH₂-wherein X is metaphenylene.

Another especially preferred group of compounds within the I Group, designated the J. Group, comprises those compounds, prodrugs thereof and pharmaceutically acceptable salts of said compounds and said prodrugs, wherein Ar is phenyl, pyridyl or imidazolyl, said phenyl, pyridyl and imidazolyl optionally substituted independently with chloro, fluoro, methyl, methoxy, difluoromethoxy, trifluoromethyl or trifluoromethoxy; Ar³ is phenyl substituted with R³¹, wherein R³¹ is (C₁-C₇)alkyl, mono-N— or di-N,N—(C₁-C₄)alkylamine, or (C₁-C₅)alkoxy, said (C, —C₇)alkyl or (C₁-C₅)alkoxy optionally mono-, di- or tri-substituted independently with hydroxy or fluoro; and Ar³ is further optionally mono- or di-substituted with chloro, fluoro, methyl, methoxy, difluoromethoxy, trifluoromethoxy or trifluoromethyl.

A preferred group of compounds within the J Group, designated the K Group, comprises those compounds, prodrugs thereof and pharmaceutically acceptable salts of said compounds and said prodrugs, wherein Q is, —(C₅-C₇)alkylene-.

Another preferred group of compounds within the J Group, designated the L Group, comprises those compounds, prodrugs thereof and pharmaceutically acceptable salts of said compounds and said prodrugs, wherein Q is —CH₂—X—CH₂— and X is phenyl optionally mono-, di- or tri-substituted with chloro, fluoro, methoxy, difluoromethoxy, trifluoromethoxy, trifluoromethyl or methyl.

An especially preferred group of compounds within the L Group are (3-(((4-butyl-benzyl)-(pyridine-3-sulfonyl)-amino)-methyl)-phenyl)-acetic acid; (3-((benzenesulfonyl-(4-butyl-benzyl)-amino)-methyl)-phenyl)-acetic acid; (3-(((4-butyl-benzyl)-(1-methyl-1H-imidazole-4-sulfonyl)-amino)-methyl)-phenyl)-acetic acid; and (3-(((4-dimethylamino-benzyl)-(pyridine-3-sulfonyl)-amino)-methyl)-phenyl)-acetic acid.

An especially preferred compound within the L Group is the compound, prodrugs thereof and pharmaceutically acceptable salts of said compounds and said prodrugs, wherein Ar is pyrid-3-yl; Z is carboxy; M is phenyl substituted at the 4-position with n-butyl; and Q is —CH₂—X—CH₂— wherein X is metaphenylene.

Another especially preferred compound within the L Group is the compound, prodrugs thereof and pharmaceutically acceptable salts of said compounds and said prodrugs, wherein Ar is phenyl; Z is carboxy; M is phenyl substituted at the 4-position with n-butyl; and Q is —CH₂—X—CH₂— wherein X is metaphenylene.

Yet another especially preferred compound within the L Group is the compound, prodrugs thereof and pharmaceutically acceptable salts of said compounds and said prodrugs, wherein Ar is 4-(1-methyl-imidazolyl); Z is carboxy; M is phenyl substituted at the 4-position with n-butyl; and Q is —CH₂—X—CH₂— wherein X is metaphenylene.

Yet another especially preferred compound within the L Group is the compound, prodrugs thereof and pharmaceutically acceptable salts of said compounds and said prodrugs, wherein Ar is pyrid-3-yl; Z is carboxy; M is phenyl substituted at the 4-position with dimethylamino; and Q is —CH₂—X—CH₂— wherein X is metaphenylene.

Another preferred group of compounds within the J Group comprises those compounds, prodrugs thereof and pharmaceutically acceptable salts of said compounds and said prodrugs, wherein Q is —(C₂-C₄)alkylene-thienyl, —(C₂-C₄)alkylene-furanyl or —(C₂-C₄)alkylene-thiazolyl.

A preferred group of compounds within the J Group, designated the M Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene, said X being optionally mono-, di- or tri-substituted with Chloro, fluoro, methoxy, difluoromethoxy, trifluoromethoxy, trifluoromethyl or methyl.

An especially preferred group of compounds within the M Group are (3-(((4-dimethylamino-benzyl)-(pyridine-3-sulfonyl)-amino)-methyl)-phenoxy)-acetic acid and (3-(((4-tert-butyl-benzyl)-(pyridine-3-sulfonyl)-amino)-methyl)-phenoxy)-acetic acid.

An especially preferred compound within the M Group is the compound, prodrugs thereof and pharmaceutically acceptable salts of said compounds and said prodrugs, wherein Ar is pyrid-3-yl; Z is carboxy; M is phenyl substituted at the 4-position with dimethylamino; and Q is —CH₂—X—O—CH₂— wherein X is metaphenylene.

Another especially preferred compound within the M Group is the compound, prodrugs thereof and pharmaceutically acceptable salts of said compounds and said prodrugs, wherein Ar is pyrid-3-yl; Z is carboxy; M is phenyl substituted at the 4-position with tert-butyl; and Q is —CH₂—X—O—CH₂— wherein X is metaphenylene.

Another preferred group of compounds within the FA Group, designated the N Group, comprises those compounds, prodrugs thereof and pharmaceutically acceptable salts of said compounds and said prodrugs, wherein G is Ar; K is (C₂-C₄) alkylene or n-propenylene; Ar is phenyl, pyrazolyl, pyridazinyl, pyrazinyl, pyridyl, imidazolyl, pyrimidyl, thienyl or thiazolyl, wherein Ar is optionally mono-, di- or tri-substituted with R³, R⁴ or R⁵; and M is Ar³, optionally mono-, di- or tri-substituted with chloro, fluoro, methyl, methoxy, difluoromethoxy, trifluoromethoxy and trifluoromethyl.

An especially preferred compound within the N Group is trans-(3-(((3-(3,5-dichloro-phenyl)-allyl)-(pyridine-3-sulfonyl)-amino)-methyl)-phenyl)-acetic acid.

An especially preferred compound within the N Group is the compound, prodrugs thereof and pharmaceutically acceptable salts of said compounds and said prodrugs, wherein K is trans-n-propenylene, said M group being attached to the 1-position of the n-propenylene and said N atom being attached to the 3-position of the n-propenylene; Ar is pyrid-3-yl; M is phenyl 3,5-disubstituted with chloro; Z is carboxy; and Q is CH₂—X—CH₂— wherein X is metaphenylene.

A preferred group of compounds within the N Group, designated the O Group, comprises those compounds, prodrugs thereof and pharmaceutically acceptable salts of said compounds and said prodrugs, wherein Ar³ is phenyl optionally substituted with chloro, fluoro, methyl, methoxy, difluoromethoxy, trifluoromethoxy or trifluoromethyl.

A preferred group of compounds within the O Group, designated the P Group, comprises those compounds, prodrugs thereof and pharmaceutically acceptable salts of said compounds and said prodrugs, wherein Q is —(C₅-C₇)alkylene-.

Another group of compounds within the O Group, designated the Q Group, comprises those compounds, prodrugs thereof and pharmaceutically acceptable salts of said compounds and said prodrugs, wherein Q is —CH₂—X—CH₂— and X is metaphenylene.

Yet another group of compounds within the O Group, designated the R Group, comprises those compounds, prodrugs thereof and pharmaceutically acceptable salts of said compounds and said prodrugs, wherein Q is —(C₂-C₄)alkylene-X— and X is furanyl, thienyl or thiazolyl. Yet another preferred group of compounds within the O Group, designated the S Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene.

Another preferred group of compounds within the FA Group, designated the T Group, comprises those compounds, prodrugs thereof and pharmaceutically acceptable salts of said compounds and said prodrugs, wherein G is Ar; K is thioethylene or oxyethylene, Ar is phenyl, pyrazolyl, pyridazinyl, pyrazinyl, pyridyl, imidazolyl, pyrimidyl, thienyl or thiazolyl, wherein Ar is optionally substituted with up to three R³, R⁴ or R⁵; and M is Ar³, optionally mono-, di- or tri-substituted with chloro, fluoro, methyl, difluoromethoxy, trifluoromethoxy or trifluoromethyl.

A preferred group of compounds within the T Group, designated the U Group, comprises those compounds, prodrugs thereof and pharmaceutically acceptable salts of said compounds and said prodrugs, wherein Ar³ is phenyl.

A preferred group of compounds within the U Group, designated the V Group, comprises those compounds, prodrugs thereof and pharmaceutically acceptable salts of said compounds and said prodrugs, wherein Q is —(C₅-C₇)alkylene-.

Another preferred group of compounds within the U Group, designated the W Group, comprises those compounds, prodrugs thereof and pharmaceutically acceptable salts of said compounds and said prodrugs, wherein Q is —CH₂—X—CH₂— and X is metaphenylene.

Another preferred group of compounds within the U Group, designated the X Group, comprises those compounds, prodrugs thereof and pharmaceutically acceptable salts of said compounds and said prodrugs, wherein Q is —(C₂-C₄)alkylene-X— and X is furanyl, thienyl or thiazolyl.

Another preferred group of compounds within the U Group, designated the Y Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene.

An especially preferred compound within the Y Group is (3-(((2-(3,5-dichloro-phenoxy)-ethyl)-(pyridine-3-sulfonyl)-amino)-methyl)-phenoxy)-acetic acid.

An especially preferred compound within the Y Group is the compound, prodrugs thereof and pharmaceutically acceptable salts of said compounds and said prodrugs, wherein K is ethylenyloxy; said M group being attached to the oxygen atom of the ethylenyloxy group and said N atom being attached to the 2-position of the ethylenyloxy group; Ar is pyrid-3-yl; M is phenyl 3,5-disubstituted with chloro; Z is carboxy and Q is —CH₂—X—O—CH₂— wherein X is a second phenyl ring and said CH₂ and OCH₂ substituents are situated in a meta substitution pattern on said second phenyl ring.

Another preferred group of compounds, designated the Z Group, comprises those compounds of Formula I, prodrugs thereof and pharmaceutically acceptable salts of said compounds and said prodrugs, wherein B is CH.

A preferred group of compounds within the Z Group comprises those compounds, prodrugs thereof and pharmaceutically acceptable salts of said compounds and said prodrugs, wherein A is CO; G is Ar, K is methylenyl, propylenyl, propenylenyl or oxyethylenyl; M is Ar³ or Ar⁴—Ar⁵; Ar³ is phenyl or pyridyl; Ar⁴ is phenyl, thienyl, pyridyl or furanyl; Ar⁵ is (C₅-C₇) cycloalkyl, phenyl, pyridyl, imidazolyl, pyrimidyl, thienyl, pyridazinyl, pyrazinyl, imidazolyl, pyrazolyl or thiazolyl; Ar is phenyl, pyrazolyl, pyridazinyl, pyrazinyl, pyridyl, imidazolyl, pyrimidyl, thienyl or thiazolyl, wherein Ar, Ar³, Ar⁴ and Ar⁵ are optionally substituted independently with up to three chloro, fluoro, methyl, difluoromethoxy, trifluoromethoxy or trifluoromethyl.

Another especially preferred group of compounds within the Z Group comprises those compounds, prodrugs thereof and pharmaceutically acceptable salts of said compounds and said prodrugs, wherein A is CO; G is Ar, K is methylenyl, propylenyl, propenylenyl or oxyethylenyl; M is Ar³ or Ar⁴—Ar⁵; Ar³ is phenyl or pyridyl; Ar⁴ is phenyl, thienyl, pyridyl or furanyl; Ar⁵ is (C₅-C₇) cycloalkyl, phenyl, pyridyl, imidazolyl, pyrimidyl, thienyl, pyridazinyl, pyrazinyl, imidazolyl, pyrazolyl or thiazolyl; Ar is phenyl, pyrazolyl, pyridazinyl, pyrazinyl, pyridyl, imidazolyl, pyrimidyl, thienyl or thiazolyl, wherein Ar, Ar³, Ar⁴ and Ar⁵ are optionally substituted independently with, up to three chloro, fluoro, methyl, difluoromethoxy, trifluoromethoxy or trifluoromethyl. Exemplary five to six membered aromatic rings optionally having one or two heteroatoms selected independently from oxygen, nitrogen and sulfur (i.e., X rings) are phenyl, furyl, thienyl, pyrrolyl, oxazolyl, thiazolyl, imidazolyl, pyrazolyl, isoxazolyl, isothiazolyl, pyridyl, pyridiazinyl, pyrimidinyl and pyrazinyl.

Exemplary partially saturated, fully saturated or fully unsaturated five to eight membered rings optionally having one to four heteroatoms selected independently from oxygen, sulfur and nitrogen (i.e., Ar, Ar¹ and Ar²) are cyclopentyl, cyclohexyl, cycloheptyl, cyclooctyl and phenyl. Further exemplary five membered rings are furyl, thienyl, 2H-pyrrolyl, 3H-pyrrolyl, pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 1,3,4-thiadiazolyl, 1,2,3,4-oxatriazolyl, 1,2,3,5-oxatriazolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered rings are 2H-pyranyl, 4H-pyranyl, pyridyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Further exemplary seven membered rings are azepinyl, oxepinyl, thiepinyl and 1,2,4-diazepinyl.

Further exemplary eight membered rings are cyclooctyl, cyclooctenyl and cyclooctadienyl.

Exemplary bicyclic rings consisting of two fused independently partially saturated, fully saturated or fully unsaturated five and/or six membered rings, taken independently, optionally having one to four heteroatoms selected independently from nitrogen, sulfur and oxygen are indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazblyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, 1,4-benzodioxan, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

Exemplary tricyclic rings consisting of three fused independently partially saturated, fully saturated or fully unsaturated five and/or six membered rings, taken independently, optionally having one to four heteroatoms selected independently from nitrogen, sulfur and oxygen are indacenyl, biphenylenyl, acenaphthylenyl, fluorenyl, phenalenyl, phenanthrenyl, anthracenyl, naphthothienyl, thianthrenyl, xanthenyl, phenoxathiinyl, carbazolyl, carbolinyl, phenanthridinyl, acridinyl, perimidinyl, phenanthrolinyl, phenazinyl, phenothiazinyl and phenoxazinyl. It will be understood that the fully saturated and all partially unsaturated forms of these rings are within the scope of this invention. Further, it will be understood that nitrogen may be substituted as the heteroatom at any position, including a bridgeghead position, in the heterocyclic rings. Further still, it will be understood that sulfur and oxygen may be substituted as the heteroatom at any non-bridgehead position within the heterocyclic rings.

By alkylene is meant saturated hydrocarbon (straight chain or branched) wherein a hydrogen atom is removed from each of the terminal carbons. Exemplary of such groups (assuming the designated length encompases the particular example) are methylene, ethylene, propylene, butylene, pentylene, hexylene and heptylene.

By alkenylene is meant a hydrocarbon containing monounsaturation in the, form of one double bond wherein said hydrocarbon, is straight chain or branched and wherein a hydrogen atom is removed from each of the terminal carbons. Exemplary of such groups (assuming the designated length encompasses the particular example) are ethenylene (or vinylene), propenylene, butenylene, pentenylene, hexenylene and heptenylene.

By alkynylene is meant a hydrocarbon containing di-unsaturation in the form of one triple bond wherein said hydrocarbon is straight chain or branched and wherein a hydrogen atom is removed from each of the terminal carbons. Exemplary of such groups (assuming the designated length encompasses the particular example) are ethynylene, propynylene, butynylene, pentynylene, hexynylene and heptynylene.

By halo is meant chloro, bromo, iodo, or fluoro.

By alkyl is meant straight chain saturated hydrocarbon or branched saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl.

By alkoxy is meant straight chain saturated alkyl or branched saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

As used herein, the term mono-N— or di-N,N-(C₁-Cₓ)alkyl . . . refers to the (C₁-Cₓ)alkyl moiety taken independently when it is di-N,N—(C₁-Cₓ)alkyl . . . (x refers to integers and is taken independently when two (C₁-Cₓ)alkyl groups are present, e.g., methylethylamino is within the scope of di-N,N—(C₁-Cₓ)alkyl).

Unless otherwise stated the “M” moieties defined above are optionally substituted (e.g., the mere listing of a substituent such as R¹ in a subgenus or dependent claim does not mean that M is always substituted with the R¹ moiety unless it is stated that the M moiety is substituted with R¹). However, in the compounds of Formula I, when K is a bond and M is phenyl, said phenyl group is substituted with one to three substituents. Additionally, in the compounds of Formula I, when Ar or Ar¹ is a fully saturated five to eight membered ring, said ring is unsubstituted.

It is to be understood that if a carbocyclic or heterocyclic moiety may be bonded or otherwise attached to a designated substrate, through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

A particularly preferred compound of Formula I is (3-(((4-tert-butyl-benzyl)-(pyridine-3-sulfonyl)-amino)-methyl)-phenoxy)-acetic acid, or a pharmaceitcally acceptable, salt or prodrug thereof, or a salt of a prodrug. A particularly preferred salt is the sodium salt.

Other EP₂ selective receptor agonists that can be used in the present invention include the prostaglandin receptor agonists disclosed in U.S. Pat. Nos. 6,288,120; and 6,124,314; and PCT published patent application WO 98/58911 (PCT/IB98/00866). A preferred EP₂ compound disclosed in U.S. Pat. No. 6,288,120 is 7-[(4-butyl-benzyl)-methanesulfonyl-amino]-heptanoic acid or a pharmaceutically acceptable salt or prodrug thereof, or a salt of a prodrug. A preferred salt of 7-[(4-butyl-benzyl)-methanesulfonyl-amino]-heptanoic acid is the monosodium salt.

Other EP₂ selective receptor agonists that can be used in the present invention include the compounds disclosed in the following: Burk, Robert M.; Holoboski, Mark; Posner, Mari F., Preparation of prostaglandin E2 analogs as EP2-receptor agonists-U.S. patent application No. 2002187961; Burk, Robert M.; Holoboski, Mark; Posner, Mari F., Preparation of prostaglandin E2 analogs as EP2-receptor agonists—U.S. Pat. No. 6,376,533; Duckworth, N.; Marshall, K.; Clayton, J. K., An investigation of the effect of the prostaglandin EP2 receptor agonist, butaprost, on the human isolated myometrium from pregnant and non-pregnant women, Journal of Endocrinology (2002), 172(2), 263-269; Tani, Kousuke; Naganawa, Atsushi; Ishida, Akiharu; Egashira, Hiromu; Odagaki, Yoshihiko; Miyazaki, Toru; Hasegawa, Tomoyuki; Kawanaka, Yasufumi; Nakai, Hisao; Ohuchida, Shuichi; Toda, Masaaki. Synthesis of a highly selective EP2-receptor agonist, Synlett (2002), (2), 239-242; Tani, Kousuke; Naganawa, Atsushi; Ishida, Akiharu; Egashira, Hiromu; Sagawa, Kenji; Harada, Hiroyuki; Ogawa, Mikio; Maruyama, Takayuki; Ohuchida, Shuichi; Nakai, Hisao; Kondo, Kigen; Toda, Masaaki. Development of a highly selective EP2-receptor agonist. Part 2. Identification of 16-Hydroxy-17,17-trimethylene 9b-chloro PGF derivatives, Bioorganic & Medicinal Chemistry (2002), 10(4), 1107-1114; Tani, Kousuke; Naganawa, Atsushi; Ishida, Akiharu; Sagawa, Kenji; Harada, Hiroyuki; Ogawa, Mikio; Maruyama, Takayuki; Ohuchida, Shuichi; Nakai, Hisao; Kondo, Kigen; Toda, Masaaki, Development of a highly selective EP2-receptor agonist. Part 1. Identification of 16-hydroxy-17,17-trimethylene PGE2 derivatives, Bioorganic & Medicinal Chemistry (2002), 10(4), 1093-1106; Michelet, Jean-Francois; Mahe, Yann; Bernard, Bruno, Use of non-prostanoic agonists of EP-2 and/or EP-4 prostaglandin receptors as cosmetic agent for reducing or stopping hair loss—European patent application EP 1175891 A1; Tani, K.; Naganawa, A.; Ishida, A.; Egashira, H.; Sagawa, K.; Harada, H.; Ogawa, M.; Maruyama, T.; Ohuchida, S.; Nakai, H.; Kondo, K.; Toda, M., Design and Synthesis of a Highly Selective EP2-Receptor Agonist, Bioorganic & Medicinal Chemistry Letters (2001), 11(15), 2025-2028; Crider, J. Y.; Sharif, N. A., Functional pharmacological evidence for EP2 and EP4 prostanoid receptors in immortalized human trabecular meshwork and non-pigmented ciliary epithelial cells., International Journal of Environmental Studies (2000), 58(1), 35-46; Crider, J. Y.; Sharif, N., A. Functional pharmacological evidence for EP2 and EP4 prostanoid receptors in immortalized human trabecular meshwork and nonpigmented ciliary epithelial cells. Journal of Ocular Pharmacology and Therapeutics (2001), 17(1), 35-46; Klimko, Peter G.; Sharif, Najam A.; Griffin, Brenda W. Prostaglandin E agonists for treatment of glaucoma—WO 0038667 A2; Woodward, David F., EP2 receptor agonists as neuroprotective agents for the eye—U.S. Pat. No. 5,877,211; Regan, John W.; Gil, Daniel W.; Woodward, David F., Cloning of a novel human prostaglandin receptor with characteristics of the pharmacologically defined EP2 subtype—U.S. Pat. No. 5,716,835; Woodward, David F. EP2-receptor agonists as agents for lowering intraocular pressure—U.S. Pat. No. 5,698,598; Woodward, David F. EP2-receptor agonists as agents for lowering intraocular pressure.—WO 9519964; Woodward, D. F.; Bogardus, A. M.; Donello, J. E.; Fairbairn, C. E.; Gil, D. W.; Kedzie, K. M.; Burke, J. A.; Kharlamb, A.; Runde, E.; et al., Molecular characterization and ocular hypotensive properties of the prostanoid EP2 receptor, Journal of Ocular Pharmacology and Therapeutics (1995), 11 (3), 447-54; Nials, Anthony T.; Vardey, Christopher J.; Denyer, Lois H.; Thomas, Malcolm; Sparrow, Susan J.; Shepherd, Gillian D.; Coleman, Robert A., AH13205, a selective prostanoid EP2-receptor agonist, Cardiovascular Drug Reviews (1993), 11(2), 165-79; and Woodward, D. F.; Protzman, C. E.; Krauss, A. H. P.; Williams, L. S., Identification of 19(R)—OH prostaglandin E2 as a selective prostanoid EP2-receptor agonist, Prostaglandins (1993), 46(4), 371-83.

The present methods can be used to treat pulmonary hypertension. Pulmonary hypertension, also known as primary pulmonary hypertension, is a disease of unknown origin that involves the medium and small pulmonary arteries and results in right ventricular failure or fatal syncope, typically 2 to 5 years after detection. Intimal hyperplasia and consequent narrowing of the vessel lumen are always present. Areas of medial hypertrophy and hyperplasia, irreversible plexiform lesions, and necrotizing arteritis (plexogenic arteriopathy) occur in more advanced cases. Those skilled in the art are familiar with the identification of patients having pulmonary hypertension.

The present methods can also be used in facilitating joint fusion. Examples of joint fusions include fusion of bones in the wrist or ankle as well as other joints. In a joint fusion, two or more bones are fused together.

The present methods can also be used to facilitate tendon and/or ligament repair. The repair can comprise the strengthening of a tendon or ligament or can comprise the reconstruction of a damaged portion of a tendon and/or ligament. Another aspect of tendon and ligament repair is to strengthen or repair the attachment of a tendon or ligament to a bone.

The present methods can also be used to reduce the occurrence of secondary fractures. A secondary fracture is a fracture subsequent to a primary fracture. Once a fracture has occurred, the present methods can be used to prevent another fracture from occurring or reduce the magnitude or complexity of any secondary fracture. For example, if a patient has a hip fracture, the present method may be used to help avoid or ameliorate the extent of a second fracture in the hip either on the same side of the hip as the first fracture or on the other side of the hip. The prevention or amelioration of damage caused by secondary fractures is also important in cases where the risk of secondary fractures is enhanced, such as in response to chemotherapy. Also, the prevention of secondary fractures in the spine and spinal stabilization are important.

The present invention also provides methods of treating avascular necrosis. Avascular necrosis is characterized by cell death in bone resulting from a compromised blood supply. The hip, femur and shoulder are commonly affected bones. Various conditions have been associated with avascular necrosis including fracture of the femoral neck, hip dislocation, decompression sickness, sickle cell disease, radiotherapy, Gaucher's disease, and corticosteroid high-dose therapy. Other conditions that have been associated with avascular necrosis include systemic lupus erythrmatosus, renal transplantation, polycythemia versa, Cushing's syndrome, Diabetes mellitus, atherosclerosis, cytotoxic chemotherapy, alcohol abuse, fatty liver, psoriasis, pancreatitis, pancreatic cancer and gout. Thus, the presence of a condition associated with avascular necrosis can be an indicator to apply the methods of the present invention to prevent or ameliorate the occurrence of avascualr necrosis in a patient.

The present invention also be used to facilitate cartilage repair, facilitate bone healing after limb transplantation, facilitate liver regeneration, facilitate wound healing, reduce the occurrence of gastric ulceration, treat hypertension, facilitate the growth of tooth enamel or finger or toe nails, treat glaucoma, treat ocular hypertension, or repair damage caused by metastatic bone disease, which conditions are well known to those skilled in the art. Patients having such conditions are easily identified by those skilled in the art.

The term “facilitating”, “facilitate”, and the like, with regard to the present methods, means to make the methods less difficult or improve the speed of the methods. For example, facilitating bone fusion means to make the procedure less difficult to accomplish or proceed more rapidly in the presence of an EP₂ selective receptor agonist than in the absence of an EP₂ selective receptor agonist.

A preferred dosage is about 0.001 to 100 mg/kg/day of an EP₂ selective receptor agonist, such as a compound of Formula I. An especially preferred dosage is about 0.01 to 10 mg/kg/day of an EP₂ selective receptor agonist, such as a compound of Formula I.

The present invention is also concerned with pharmaceutical compositions comprising an EP₂ selective receptor agonist, such as a compound of Formula I, and a carrier, solvent, diluent and the like.

Another aspect of this invention is directed to combinations of an EP₂ selective receptor agonist, such as a compound of Formula I, and other therapeutically useful compounds.

In one embodiment, the combinations of this invention comprise a therapeutically effective amount of a first compound, said first compound being an EP₂ selective receptor agonist, such as a compound of Formula I; and a therapeutically effective amount of a second compound, the second compound being an anti-resorptive agent such as an estrogen agonist/antagonist or a bisphosphonate. An estrogen agonist/antagonist is also called a selective estrogen receptor modulator (SERM).

It is noted that when compounds are discussed herein, it is contemplated that the compounds may be administered to a patient as a therapeutically acceptable salt, prodrug, or a salt of a prodrug. All such variations are intended to be included in the invention.

Another aspect of this invention is a kit comprising:
a. an amount of an EP₂ selective receptor agonist, such as a compound of Formula I, and a pharmaceutically acceptable carrier or diluent in a first unit dosage form;
b. an amount of an anti-resorptive agent, and a pharmaceutically acceptable carrier or diluent in a second unit dosage form; and
c. a container.

Yet another aspect of this invention is directed to pharmaceutical compositions or kits comprising an EP₂ selective receptor agonist, such as a compound of Formula I, and another bone anabolic agent (although the other bone anabolic agent may be another EP₂ selective receptor agonist, such as a different compound of Formula I). Such compositions comprise a therapeutically effective amount of a first compound, said first compound being an EP₂ selective receptor agonist, such as a compound of Formula I; and a therapeutically effective amount of a second compound, said second compound being another bone anabolic agent.

The first compound can administered at the same time as the second compound in the same dosage form or in different dosage forms. Alternatively, the first compound and second compound can be administered at different times. Moreover, the combinations of the present invention can comprise more than two compounds. For example, two EP₂ selective receptor agonists and an anti-repsorptive or bone anabolic compound can be administered to a patient.

Another aspect of this invention is a kit comprising:
a. an amount of an EP₂ receptor selective agonist, such as a compound of Formula I, in a first unit dosage form;
b. an amount of a second compound, said second compound being another bone anabolic agent in a second unit dosage form; and
c. a container.

Preferred bone anabolic agents include IGF-1, prostaglandins, prostaglandin agonists/antagonists, sodium fluoride, parathyroid hormone (PTH), active fragments of parathyroid hormone, parathyroid hormone related peptides and active fragments and analogues of parathyroid hormone related peptides, growth hormones or growth hormone secretagogues and the pharmaceutically acceptable salts or prodrugs thereof or a salt of a prodrug.

Since the present invention has an aspect that relates to a combination of active ingredients, which may be administered separately, the invention also relates to combining separate pharmaceutical compositions in kit form. The kit comprises two separate pharmaceutical compositions: an EP₂ selective receptor agonist, such ap a compound of Formula I and a second compound as described above. The kit comprises container means for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . .. Second Week, Monday, Tuesday, . . . ” etc. Other variations of memory aids will be readily apparent. A “daily dose” can be a single tablet or capsule or several tablets or capsules to be taken on a given day. Also, a daily dose of a Formula I compound, a prodrug thereof or a pharmaceutically acceptable salt of said compound or said prodrug can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Preferred estrogen agonists/antagonists of the present invention include the compounds described in U.S. Pat. No. 5,552,412. Those compounds are described by the formula designated herein as formula (I) given below: 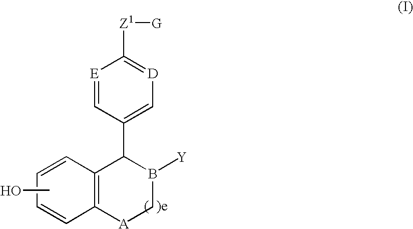
wherein:
A is selected from CH₂ and NR;
B, D and E are independently selected from CH and N;
Y is
   (a) phenyl, optionally substituted with 1-3 substituents independently selected from R⁴;
   (b) naphthyl, optionally substituted with 1-3 substituents independently selected from R⁴;
   (c) C₃-C₈ cycloalkyl, optionally substituted with 1-2 substituents independently selected from R
   (d) C₃-C₈ cycloalkenyl, optionally substituted with 1-2 substituents independently selected from R⁴;
   (e) a five membered heterocycle containing up to two heteroatoms selected from the group consisting of —O—, —NR²— and —S(O)ₙ—, optionally substituted with 1-3 substituents independently selected from R⁴;
   (f) a six membered heterocycle containing up to two heteroatoms selected from the group consisting of —O—, —NR²— and —S(O)ₙ— optionally substituted with 1-3 substituents independently selected from R⁴; or
   (g) a bicyclic ring system consisting of a five or six membered heterocyclic ring fused to a phenyl ring, said heterocyclic ring containing up to two heteroatoms selected from the group consisting of —O—, —NR²— and —S(O)ₙ—, optionally substituted with 1-3 substituents independently selected from R⁴;
Z¹ is
   (a) —(CH₂)ₚW(CH₂)_{q}—;
   (b)—Q(CH₂)ₚCR⁵R⁵—;
   (c) —O(CH₂)ₚW(CH₂)_{q}—;
   (d) —OCHR²CHR³—; or
   (e) —SCHR²CHR³—;
G is 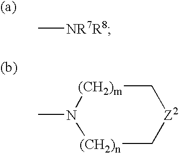
wherein n is 0, 1 or 2; m is 1, 2 or 3; Z² is —NH—, —O—, —S—, or —CH₂—; optionally fused on adjacent carbon atoms with one or two phenyl rings and, optionally independently substituted on carbon with one to three substituents and, optionally, independently on nitrogen with a chemically suitable substituent selected from R⁴; or
   (c) a bicyclic amine containing five to twelve carbon atoms, either bridged or fused and optionally substituted with 1-3 substituents independently selected from R⁴; or
Z¹ and G in combination may be 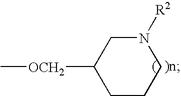
W is 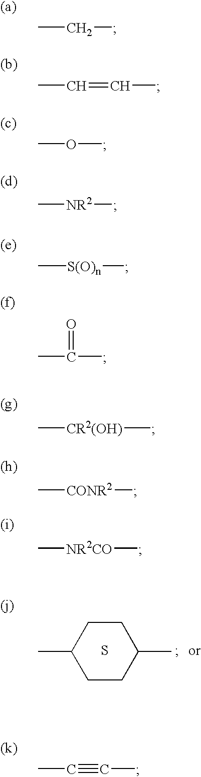
R is hydrogen or C₁-C₆ alkyl,
R² and R³ are independently
   (a) hydrogen; or
   (b) C₁-C₄ alkyl;
R⁴ is
   (a) hydrogen;
   (b) halogen;
   (c) C₁-C₆ alkyl;
   (d) C₁-C₄ alkoxy;
   (e) C₁-C₄ acyloxy;
   (f) C₁-C₄ alkylthio;
   (g) C₁-C₄ alkylsulfinyl;
   (h) C₁-C₄ alkylsulfonyl;
   (i) hydroxy (C₁-C₄)alkyl;
   (j) aryl (C₁-C₄)alkyl;
   (k) —CO₂H;
   (l) —CN;
   (m) —CONHOR;
   (n) —SO₂NHR;
   (O) —NH₂;
   (p) C₁-C₄ alkylamino;
   (q) C₁-C₄ dialkylamino;
   (r) —NHSO₂R;
   (s) —NO₂;
   (t) -aryl; or
   (u) —OH;
R⁵ and R⁶⁵ are independently C₁-C₈ alkyl or together form a C₃-C₁₀ carbocyclic ring;
R⁷ and R⁸ are independently
   (a) phenyl;
   (b) a C₃-C₁₀ carbocyclic ring, saturated or unsaturated;
   (c) a C₃-C₁₀ heterocyclic ring containing up to two heteroatoms, selected from —O—, —N— and —S—;
   (d) H;
   (e) C₁-C₆ alkyl; or
   (f) form a 3 to 8 membered nitrogen containing ring with R⁵ or R⁶;
R⁷ and R⁸ in either linear or ring form may optionally be substituted with up to three substituents independently selected from C₁-C₆ alkyl, halogen, alkoxy, hydroxy and carboxy;
a ring formed by R⁷ and R⁸ may be optionally fused to a phenyl ring;
e is 0, 1 or
m is 1, 2 or 3;
n is 0, 1 or 2;
p is 0, 1, 2 or 3;
q is 0, 1, 2 or 3;
and optical and geometric isomers thereof; and nontoxic pharmaceutically acceptable acid addition salts, N-oxides, esters, quaternary ammonium salts and prodrugs thereof.

Additional preferred estrogen agonists/antagonists are disclosed in U.S. Pat. No. 5,552,412 and are described by the formula designated herein as formula (IA): 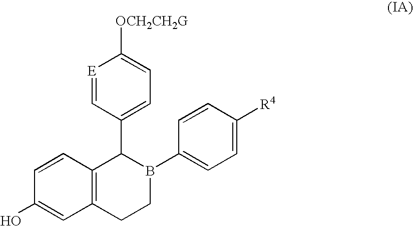
wherein G is 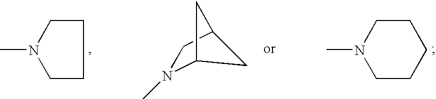
R⁴ is H, OH, F, or Cl; and B and E are independently selected from CH and N, and optical and geometric isomers thereof; and nontoxic pharmaceutically acceptable acid addition salts, N-oxides, esters, quaternary ammonium salts and prodrugs thereof.

Especially preferred estrogen agonists/antagonists for the methods of the invention are:
cis-6-(4-fluoro-phenyl)-5-[4-(2-piperidin-1-yl-ethoxy)-phenyl]-5,6,7,8-tetrahydro-naphthalene-2-ol;
(−)-cis-6-phenyl-5-[4-(2-pyrrolidin-1-yl-ethoxy)-phenyl]-5,6, 7,8-tetrahydro-naphthalene-2-ol; cis-6-phenyl-5-[4-(2-pyrrolidin-1-yl-ethoxy)-phenyl]-5,6,7,8-tetrahydronaphthalene-2-ol;
cis-1-[6′-pyrrolidinoethoxy-3′-pyridyl]-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,4-tetrahydroisoquinoline;
cis-6-(4-hydroxyphenyl)-5-[4-(2-piperidin-1-yl-ethoxy)-phenyl]-5,6,7,8-tetrahydro-naphthalene-2-ol;
1-(4′-pyrrolidinoethoxyphenyl)-2-phenyl-6-hydroxy-1,2,3,4-tetrahydroisoquinoline and pharmaceutically acceptable salts thereof.

An especially preferred salt of (−)-cis-6-phenyl-5-[4-(2-pyrrolidin-1-yl-ethoxy)-phenyl]-5,6,7,8-tetrahydro-naphthalene-2-ol is the D-tartrate salt.

Other preferred estrogen agonists/antagonists are disclosed in U.S. Pat. No. 5,047,431. The structure of these compounds are described by the formula designated herein as formula (II) below: 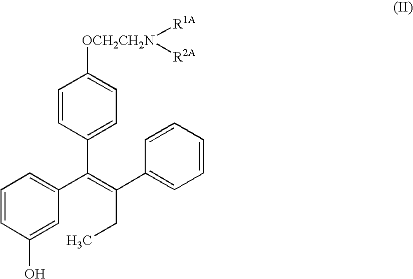
wherein
R^{1A} and R^{2A} may be the same or different and are either H, methyl, ethyl or a benzyl group; and optical or geometric isomers thereof; and pharmaceutically acceptable salts, N-oxides, esters, quaternary ammonium salts, and prodrugs thereof.

Additional preferred estrogen agonists/antagonists are the compounds disclosed in U.S. Pat. No. 4,536,516; 4-hydroxy tamoxifen (i.e., tamoxifen wherein the 2-phenyl moiety has a hydroxy group at the 4 position) and other compounds as disclosed in U.S. Pat. No. 4,623,660; raloxifene: (methanone, [6-hydroxy-2-(4-hydroxyphenyl)benzo[b]thien-3-yl][4-[2-(1-piperidinyl)ethoxy]phenyl]-, hydrochloride) and other compounds as disclosed in U.S. Pat. Nos. 4,418,068; 5,393,763; 5,457,117; 5,478,847 and 5,641,790; toremifene: (ethanamine, 2-[4-(4-chloro-1,2-diphenyl-1-butenyl)phenoxy]-N,N-dimethyl-, (Z)-, 2-hydroxy-1,2,3-propanetricarboxylate (1:1) and other compounds as disclosed in U.S. Pat. Nos. 4,696,949 and 4,996,225; centchroman: 1-[2-[[4-(-methoxy-2,2, dimethyl-3-phenyl-chroman-4-yl)-phenoxy]-ethyl]-pyrrolidine and other compounds as disclosed in U.S. Pat. No. 3,8,22,287; idoxifene: pyrrolidine, 1-[-[4-[[1-(4-iodophenyl)-2-phenyl-1-butenyl]phenoxy]ethyl], and other compounds as disclosed in U.S. Pat. No. 4,839,155; 6-(4-hydroxy-phenyl)-5-[4-(2-piperidin-1-yl-ethoxy)-benzyl]-naphthalen-2-ol and other compounds as disclosed in U.S. Pat. No. 5,484,795; and {4-[2-(2-aza-bicyclo[2.2.1]hept-2-yl)-ethoxy]-phenyl}-[6-hydroxy-2-(4-hydroxy-phenyl)-benzo[b]thiophen-3-yl]-methanone and other compounds as disclosed in published international patent application WO 95/10513. Other preferred compounds include GW 5638 and GW 7604, the synthesis of which is described in Willson et al., J. Med. Chem., 1994;37:1550-1552.

Further preferred estrogen agonists 1 antagonists include EM-652 (as shown in the formula designated herein as formula (III) and EM-800 (as shown in the formula designated herein as formula (IV)). The synthesis of EM-652 and EM-800 and the activity of various enantiomers is described in Gauthier et al., J. Med. Chem., 1997;40:2117-2122.

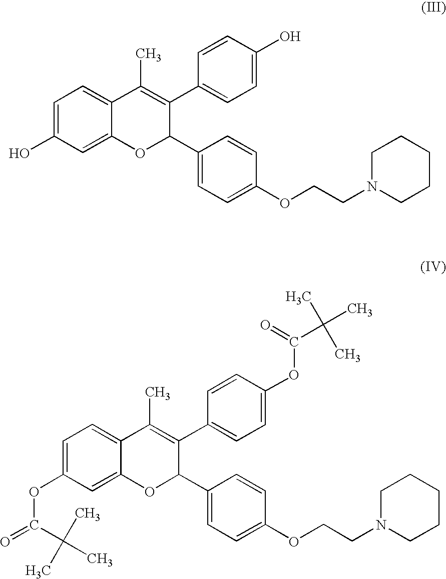

Further preferred estrogen agonists/antagonists include TSE 424 and other compounds disclosed in U.S. Pat. No. 5,998,402, U.S. Pat. No. 5,985,910, U.S. Pat. No. 5,780,497, U.S. Pat. No. 5,880,137, and European Patent Application EP 0802183 A1 including the compounds described by the formulae designated herein as formulae V and VI, below: 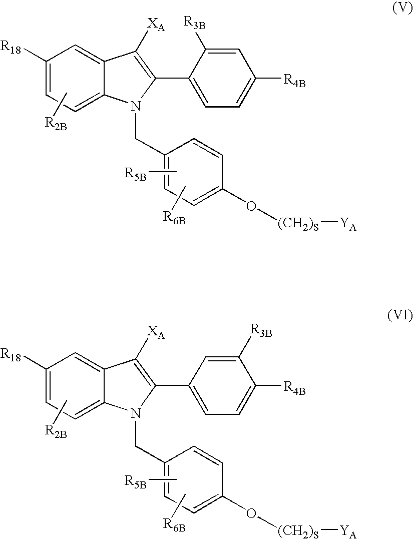
wherein:
R_{1B} is selected from H, OH or the C₁-C₁₂ esters (straight chain or branched) or C₁-C₁₂ (straight chain or branched or cyclic) alkyl ethers thereof, or halogens; or C₁-C₄ halogenated ethers including trifluoromethyl ether and trichloromethyl ether.
R_{2B}, R_{3B}, R_{4B}, R_{5B}, and R_{6B} are independently selected from H, OH or the C₁-C₁₂ esters (straight chain or branched) or C₁-C₁₂ alkyl ethers (straight chain or branched or cyclic) thereof, halogens, or C₁-C₄ halogenated ethers including trifluoromethyl ether and trichloromethyl ether, cyano, C₁-C₆ alkyl (straight chain or branched), or trifluoromethyl;
X_{A} is selected from H, C₁-C₆ alkyl, cyano, nitro, trifluoromethyl, and halogen;
s is 2 or 3;
Y_{A} is selected from:
a) the moiety: 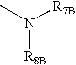
wherein R_{7B} and R_{8B} are independently selected from the group of H, C₁-C₆ alkyl, or phenyl optionally substituted by CN, C₁-C₆ alkyl (straight chain or branched), C₁-C₆ alkoxy (straight chain or branched), halogen, —OH, —CF₃, or —OCF₃;
b) a five-membered saturated, unsaturated or partially unsaturated heterocycle containing up to two heteroatoms selected from the group consisting of —O—, —NH—, —N(C₁-C₄ alkyl)-, —N═, and —S(O)ᵤ—, wherein u is an integer of from 0-2, optionally substituted with 1′-3 substituents independently selected from the group consisting of hydrogen, hydroxyl, halo, C₁-C₄ alkyl, trihalomethyl, C₁-C₄ alkoxy, trihalomethoxy, C₁-C₄ acyloxy, C₁-C₄ alkylthio, C₁-C₄ alkylsulfinyl, C₁-C₄ alkylsulfonyl, hydroxy (C₁-C₄)alkyl, —CO₂H, —CN, —CONHR_{1B}, —NH₂, C₁-C₄ alkylamino, di(C₁-C₄)alkylamino, —NHSO₂R_{1B}, —NHCOR_{1B}, —NO₂, and, phenyl optionally substituted with 1-3 (C₁-C₄)alkyl;
c) a six-membered saturated, unsaturated or partially unsaturated heterocycle containing up to two heteroatoms selected from the group consisting of —O—, —NH—, —N(C₁-C₄ alkyl)-, —N═, and —S(O)ᵤ—, wherein u is an integer of from 0-2, optionally substituted with 1-3 substituents independently selected from the group consisting of hydrogen, hydroxyl, halo, C₁-C₄ alkyl, trihalomethyl, C₁-C₄ alkoxy, trihalomethoxy, C₁-C₄ acyloxy, C₁-C₄ alkylthio, C₁-C₄ alkylsulfinyl, C₁-C₄ alkylsulfonyl, hydroxy (C₁-C₄)alkyl, —CO₂H, —CN, —CONHR₁, —NH₂, C₁-C₄ alkylamino, di(C₁-C₄)alkylamino, —NHSO₂R_{1B}, —NHCOR_{1B}, —NO₂, and phenyl optionally substituted with 1-3 (C₁-C₄)alkyl;
d) a seven-membered saturated, unsaturated or partially unsaturated heterocycle containing up to two heteroatoms selected from the group consisting of —O—, —NH—, —N(C₁-C₄ alkyl)-, —N═, and —S(O)ᵤ—, wherein u is an integer of from 0-2, optionally substituted with 1-3 substituents independently selected from the group consisting of hydrogen, hydroxyl, halo, C₁-C₄ alkyl, trihalomethyl, C₁-C₄ alkoxy, trihalomethoxy, C₁-C₄ acyloxy, C₁-C₄ alkylthio, C₁-C₄ alkylsulfinyl, C₁-C₄ alkylsulfonyl, hydroxy (C₁-C₄)alkyl, —CO₂H, —CN, —CONHR_{1B}, —NH₂, C₁-C₄ alkylamino, di(C₁-C₄)alkylamino, —NHSO₂R_{1B}, —NHCOR_{1B}, —NO₂, and phenyl optionally substituted with 1-3 (C₁-C₄)alkyl; or
e) a bicyclic heterocycle containing from 6-12 carbon atoms either bridged or fused and containing up to two heteroatoms selected from the group consisting of —O—, —NH—, —N(C₁-C₄ alkyl)-, and —S(O)ᵤ—, wherein u is an integer of from 0-2, optionally substituted with 1-3 substituents independently selected from the group consisting of hydrogen, hydroxyl, halo, C₁-C₄ alkyl, trihalomethyl, C₁-C₄ alkoxy, trihalomethoxy, C₁-C₄ acyloxy, C₁-C₄ alkylthio, C₁-C₄ alkylsulfinyl, C₁-C₄ alkylsulfonyl, hydroxy (C₁-C₄)alkyl, —CO₂H—, —CN—, —CONHR_{1B}, —NH₂, —N═, C₁-C₄ alkylamino, di(C₁-C₄)alkylamino, —NHSO₂RIB, —NHCOR_{1B}, —NO₂, and phenyl optionally substituted with 1-3 (C₁-C₄) alkyl; and optical and geometric isomers thereof; and nontoxic pharmaceutically acceptable acid addition salts, N-oxides, esters, quaternary ammonium salts, and prodrugs thereof.

Preferred compounds of this invention are those having the general structures V or VI, above, wherein:
R_{1B} is selected from H, OH or the C₁-C₁₂ esters or alkyl ethers thereof, and halogen;
R_{2B}, R_{3B}, R_{4B}, R_{5B}, and R_{6B} are independently selected from H, OH or the C₁-C₁₂ esters or alkyl ethers thereof, halogen, cyano, C₁-C₆ alkyl, or trihalomethyl, preferably trifluoromethyl, with the proviso that, when R_{1B} is H, R_{2B} is not OH;
X_{A} is selected from H, C₁-C₆ alkyl, cyano, nitro, trifluoromethyl, and halogen;
Y_{A} is the moiety: 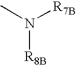
R_{7B} and R_{8B} are selected independently from H, C₁-C₆ alkyl, or combined by —(CH₂)_{w}—, wherein w is an integer of from 2 to 6, so, as to form a ring, the ring being optionally substituted by up to three substituents selected from the group of hydrogen, hydroxyl, halo, C₁-C₄ alkyl, trihalomethyl, C₁-C₄ alkoxy, trihalomethoxy, C₁-C₄ alkylthio, C₁-C₄ alkylsulfinyl, C₁-C₄ alkylsulfonyl, hydroxy (C₁-C₄)alkyl, —CO₂H, —CN, —CONH(C₁-C₄alkyl), —NH₂, C₁-C₄ alkylamino, Q₁-C₄ dialkylamino, —NHSO₂(C₁-C₄alkyl), —CO(C₁-C₄alkyl), and —NO₂; and optical and geometric isomers thereof; and nontoxic pharmaceutically acceptable acid addition salts, N-oxides, esters, quaternary ammonium salts, and prodrugs thereof.

The rings formed by a concatenated R_{7B} and R_{8B}, mentioned above, may include, but are not limited to, aziridine, azetidine, pyrrolidine, piperidine, hexamethyleneamine or heptamethyleneamine rings.

Preferred compounds of structural formulas V and VI, above, are those wherein R_{1B} is OH; R_{2B}-R_{6B} are as defined above; X_{A} is selected from the group of Cl, NO₂, CN, CF₃, or CH₃; Y_{A} is the moiety 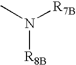
and R_{7B} and R_{8B} are concatenated together as —(CH₂)ₜ—, wherein t is an integer of from 4 to 6, to form a ring optionally substituted by up to three subsituents selected from the group of hydrogen, hydroxyl, halo, C₁-C₄ alkyl, trihalomethyl, C₁-C₄ alkoxy, trihalomethoxy, C₁-C₄ alkylthio, C₁-C₄ alkylsulfinyl, C₁-C₄ alkylsulfonyl, hydroxy (C₁-C₄)alkyl, —CO₂H, —CN, —CONH(C₁-C₄)alkyl, —NH₂, C₁-C₄ alkylamino, di(C₁-C₄)alkylamino, —NHSO₂(C₁-C₄)alkyl, —NHCO(C₁-C₄)alkyl, and —NO₂; and optical and geometric isomers thereof; and nontoxic pharmaceutically acceptable acid addition salts, N-oxides, esters, quaternary ammonium salts, and prodrugs thereof.

Another preferred compound is TSE-424 as described by the formula designated herein as formula (Va) below:

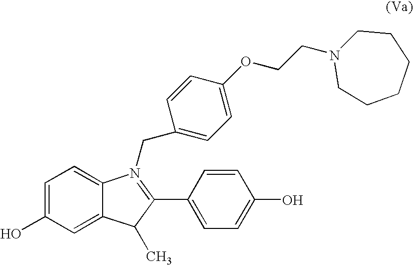

Another estrogen agonist/antagonist that can be used in the combination, aspect of the present invention is arzoxifene, which is disclosed in U.S. Pat. No. 5,723,474.

A particularly preferred combination of an EP₂ selective receptor agonist and an estrogen agonist/antagonist is (3-(((4-tert-butyl-benzyl)-(pyridine-3-sulfonyl)-amino)-methyl)-phenoxy)-acetic acid and (−)-cis-6-phenyl-5-(4-(2-pyrrolidin-1-yl-ethoxy)-phenyl)-5,6,7,8-tetrahydro-naphthalene-2-ol. In a more preferred combination, the (3-(((4-tert-butyl-benzyl)-(pyridine-3-sulfonyl)-amino)-methyl)-phenoxy)-acetic acid is in the form of the sodium salt, and the (−)-cis-6-phenyl-5-(4-(2-pyrrolidin-1-yl-ethoxy)-phenyl)-5,6,7,8-tetrahydro-naphthalene-2-ol is in the form of the D-tartrate salt.

Preferred bisphosphonates include, tiludronic acid, alendronic acid, zoledronic acid, ibandronic acid, risedronic acid, etidronic acid, clodronic acid, and pamidronic acid and their pharmaceutically acceptable salts or prodrugs or salts of the prodrugs.

It will be recognized that prodrugs and pharmaceutically acceptable salts may be formed from the compounds of this invention. All of such prodrugs and pharmaceutically acceptable salts so formed are within the scope of this invention. Particularly preferred salt forms of the estrogen agonists/antagonists include, raloxifene hydrochloride, tamoxifen citrate, toremifene citrate, and lasofoxifene tartrate.

Those skilled in the art will recognize that anti-resorptive agents such as progestins, polyphosphonates, bisphosphonate(s), estrogen agonists/antagonists, estrogen, estrogen/progestin combinations, Premarin® (conjugated estrogens), estrone, estriol or 1 7α- or 17β-ethynyl estradiol may be used in conjunction with the EP₂ selective receptor agonists in the present methods.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary polyphosphonates include polyphosphonates of the type disclosed in U.S. Pat. No. 3,683,080. Preferred polyphosphonates are geminal diphosphonates (also referred to as bis-phosphonates). Tiludronate disodium is an especially preferred polyphosphonate. Ibandronic acid is an especially preferred polyphosphonate. Alendronate is an especially preferred polyphosphonate. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1, 1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid,N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

Any prostaglandin may be used in combinaiton with an EP₂ selective receptor agonist in the present methods. The term prostaglandin refers to compounds that are analogs of the natural prostaglandins PGD₁, PGD₂, PGE₂, PGE₁ and PGF₂. These compounds bind to the prostaglandin receptors. Such binding is readily determined by those skilled in the art of standard assays (e.g., An S. et al., Cloning and Expression of the EP₂ Subtype of Human Receptors for Prostaglandin E₂, Biochemical and Biophysical Research Communications, 1993, 197(1):263-270).

Prostaglandins are alicyclic compounds related to the basic compound prostanoic acid. The carbon atoms of the basic prostaglandin are numbered sequentially from the carboxylic carbon atom through the cyclopentyl ring to the terminal carbon atom on the adjacent side chain. Normally the adjacent side chains are in the trans orientation. The presence of an oxo group at C-9 of the cyclopentyl moiety is indicative of a prostaglandin within the E class while PGE₂ contains a trans unsaturated double bond at the C₁₃-C₁₄ and a cis double bond at the C₅-C₆ position.

A variety of prostaglandins are described and referenced below. However, other prostaglandins will be known to those skilled in the art. Exemplary prostaglandins are disclosed in U.S. Pat. Nos. 4,171,331 and 3,927,197.

Norrdin et al., The Role of Prostaglandins in Bone In Vivo, Prostaglandins Leukotriene Essential Fatty Acids 41, 139-150, 1990 is a review of bone anabolic prostaglandins.

Any prostaglandin agonist/antagonist may be used as the second compound in certain aspects of this invention. The term prostaglandin agonist/antagonist refers to compounds which bind to prostaglandin receptors (e.g., An S. et al., Cloning and Expression of the EP₂ Subtype of Human Receptors for Prostaglandin E₂, Biochemical and Biophysical Research Communications, 1993, 197(1):263-270) and mimic the action of prostaglandin in vivo. Such actions are readily determined by those skilled in the art of standard assays. Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296. A variety of these compounds are described and referenced below. However, other prostaglandin agonists/antagonists will be known to those skilled in the art. Exemplary prostaglandin agonists/antagonists are disclosed as follows: U.S. Pat. No. 3,932,389 discloses 2-descarboxy-2-(tetrazol-5-yl)-11-desoxy-15-substituted-omega-pentanorprostaglandins; U.S. Pat. No. 4,018,892 discloses 16-aryl-13,14-dihydro-PGE₂ p-biphenyl esters; U.S. Pat. Nos. 4,219,483 and 4,132,847 discloses 2,3,6-substituted-4-pyrones; U.S. Pat. Nos. 4,000,309 and 3,982,016 discloses 16-aryl-13,14-dihydro-PGE₂ p-biphenyl esters; U.S. Pat. No. 4,621,100 discloses substituted cyclopentanes; and U.S. Pat. No. 5,216,183 discloses cyclopentanones.

Sodium fluoride may be used as the second compound in certain aspects of this invention. The term sodium fluoride refers to sodium fluoride in all its forms (e.g., slow release sodium fluoride, sustained release sodium fluoride). Sustained release sodium fluoride is disclosed in U.S. Pat. No. 4,904,478. The activity of sodium fluoride is readily determined by those skilled in the art of biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296).

Any parathyroid hormone (PTH) may be used as the second compound in certain aspects of this invention. The term parathyroid hormone refers to parathyroid hormone, fragments or metabolites thereof and structural analogs thereof which can stimulate bone formation and increase bone mass. Also included are parathyroid hormone related peptides and active fragments and analogs of parathyroid related peptides (see PCT publication no. WO 94/01460). A variety of these compounds are described and referenced below. However, other parathyroid hormones will be known to those skilled in the art. Exemplary parathyroid hormones are disclosed in the following references. “Human Parathyroid Peptide Treatment of Vertebral Osteoporosis”, Osteoporosis Int., 3, (Supp 1):199-203.

“PTH 1-34 Treatment of Osteoporosis with Added Hormone Replacement Therapy: Biochemical, Kinetic and Histological Responses” Osteoporosis Int. 1:162-170.

Any growth hormone or growth hormone secretagogue may be used as the second compound in certain aspects of this invention. The term growth hormone secretagogue refers to a compound which stimulates the release of growth hormone or mimics the action of growth hormone (e.g., increases bone formation leading to increased bone mass). Such actions are readily determined by those skilled in the art of standard assays well known to those of skill in the art. A variety of these compounds are disclosed in the following published PCT patent applications: WO 95/14666; WO 95/13069; WO 94/19367; WO 94/13696; and WO 95/34311. However, other growth hormones or growth hormone secretagogues will be known to those skilled in the art.

In particular a preferred growth hormone secretagogue is N-[1(R)-[1,2-Dihydro-1-methanesulfonylspiro[3H-indole-3,4′-piperidin)-1′-yl)carbonyl]-2-(phenylmethyloxy)ethyl]-2-amino-2-methylpropanamide:MK-677.

Other preferred growth hormone secretagogues include
2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide or its L-tartaric acid salt;
2-amino-N-(1-(R)-benzyloxymethyl-2-(3a-(R)-(4-fluoro-benzyl)-2-methyl-3-oxo-2,3,3a,4,6,7-hexahydro-pyrazolo[4,3-c]pyridin-5-yl)-2-oxo-ethyl)isobutyramide;
2-amino-N-(2-(3a-(R)-benzyl-3-oxo-2,3,3a,4,6,7-hexahydro-pyrazolo[4,3-c]pyridin-5-yl)-1-(R)benzyloxymethyl-2-oxo-ethyl)isobutyramide; and
2-amino-N-(1-(2,4-difluoro-benzyloxymethyl)-2-oxo-2-(3-oxo-3a-pyridin-2-ylmethyl-2-(2,2,2-trifluoro-ethyl)-2,3,3a,4,6,7-hexahydro-pyrazolo[4,3-c]pyridin-5-yl)-ethyl)-2-methyl-propionamide.

The term “treating”, “treat” or “treatment” as used herein includes preventative (e.g., prophylactic), palliative and curative treatment.

By “pharmaceutically acceptable” it is meant the carrier, diluent, excipients, and/or salts or prodrugs must be compatible with the other ingredients of the formulation, and not deleterious to the recipient thereof.

The term “prodrug” means a compound that is transformed in vivo to yield a compound of the present invention. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, when a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, when a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

When a compound of the present invention comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently (C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein Y^{X} is H, (C₁-C₆)alkyl or benzyl), C(OY^{X0}) Y^{X1} wherein Y^{X0} is (C₁-C₄) alkyl and Y^{X1} is (C₁-C₆)alkyl, carboxy(C₁-C₆)alkyl, amino(C₁-C₄)alkyl or mono-N— or di-N,N—(C₁-C₆)alkylaminoalkyl, C(Y^{X2}) Y^{X3} wherein Y^{X2} is H or methyl and Y^{X3} is mono-N— or di-N,N—(C₁-C₆)alkylamino, morpholino, piperidin-1-yl or pyrrolidin-1-yl.

The expression “pharmaceutically acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

As used herein, the expressions “reaction inert solvent” and “inert solvent” refers to a solvent that does not interact with starting materials, reagents, intermediates or products in a manner which adversely affects the yield of the desired product.

It will be recognized that the compounds of this invention can exist in radiolabelled form, i.e., said compounds may contain one or more atoms containing an atomic mass or mass number different from the atomic mass or mass number ordinarily found in nature. Radioisotopes of hydrogen, carbon, phosphorous, fluorine and chlorine include ³H, ¹⁴C, ³²P, ³⁵S, ¹⁸F and ³⁶Cl, respectively. Compounds of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radiolabelled compounds of this invention can generally be prepared of methods well known to those skilled in the art. Conveniently, such radiolabelled compounds can be prepared by carrying out the procedures disclosed in the above Schemes and/or in the Examples and Preparations below by substituting a readily available radiolabelled reagent for a non-radiolabelled reagent.

It will be recognized by persons of ordinary skill in the art that some of the compounds of this invention have at least one asymmetric carbon atom and therefore are enantiomers or diastereomers. Diasteromeric mixtures can be separated into their individual diastereomers on the basis of their physicochemical differences by methods known per se as, for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing, including both chemical hydrolysis methods and microbial lipase hydrolysis methods, e.g., enzyme catalyzed hydrolysis) the individual diastereomers to the corresponding pure enantiomers. All such isomers, including diastereomers, enantiomers and mixtures thereof are considered as part of this invention. Also, some of the compounds of this invention are atropisomers (e.g., substituted biaryls) and are considered as part of this invention.

In addition, when the compounds of this invention, including the compounds of Formula I, the anti-resorptive agents, bone anabolic agents, prostaglandin agonists/antagonists, parathyroid hormones, growth hormones and growth hormone secretagogues, form hydrates or solvates, they are also within the scope of the invention.

Administration of the compounds of this invention can be via any method that delivers a compound of this invention systemically and/or locally. These methods include oral routes, parenteral, intraduodenal routes, etc. Generally, the compounds of this invention are administered orally, but parenteral administration (e.g., intravenous, intramuscular, transdermal, subcutaneous, rectal or intramedullary) may be utilized, for example, where oral administration is inappropriate for the target or where the patient is unable to ingest the drug.

The compounds of this invention may also be applied locally to a site in or on a patient in a suitable carrier or diluent, optionally in combination with one or more of the anabolic agents or anti-resorptive agents described above.

The amount and timing of compounds administered will, of course, be dependent on the subject being treated, on the severity of the affliction, on the manner of administration and on the judgment of the prescribing physician. Thus, because of patient to patient variability, the dosages given herein are guidelines and the physician may titrate doses of the drug to achieve the treatment that the physician considers appropriate for the patient. In considering the degree of treatment desired, the physician must balance a variety of factors such age of the patient, presence of preexisting disease, as well as presence of other diseases (e.g., cardiovascular disease).

In general an effective dosage for the anabolic agents used in this invention described above is in the range of 0.001 to 100 mg/kg/day, preferably 0.01 to 50 mg/kg/day.

The following paragraphs provide preferred dosage ranges for various anti-resorptive agents.

In general, an effective dosage for an anti-resorptive agent is about 0.001 mg/kg/day to about 20 mg/kg/day.

In general, an effective dosage for progestins is about 0.1 to 10 mg per day; the preferred dose is about 0.25 to 5 mg per day.

In general, an effective dosage for polyphosphonates is determined by its potency as a bone resorption inhibiting agent of standard assays.

Ranges for the daily administration of some polyphosphonates are about 0.001 mg/kg/day to about 20 mg/kg/day.

In general an effective dosage for the treatment of this invention, for example the bone resorption treatment of this invention, for the estrogen agonists/antagonists of this invention is in the range of 0.01 to 200 mg/kg/day, preferably 0.5 to 100 mg/kg/day.

In particular, an effective dosage for raloxifene is in the range of 0.1 to 100 mg/kg/day, preferably 0.1 to 10 mg/kg/day.

In particular, an effective dosage for tamoxifen is in the range of 0.1 to 100 mg/kg/day, preferably 0.1 to 5 mg/kg/day.

In particular, an effective dosage for 2-(4-methoxy-phenyl)-3-[4-(2-piperidin-1-yl-ethoxy)-phenoxy]-benzo[b]thiophen-6-ol is 0.001 to 1 mg/kg/day.

In particular, an effective dosage for
cis-6-(4-fluoro-phenyl)-5-(4-(2-piperidin-1-yl-ethoxy)-phenyl)-5,6,7,8-tetrahydro-naphthalene-2-ol;
(−)-cis-6-phenyl-5-(4-(2-pyrrolidin-1-yl-ethoxy)-phenyl)-5,6,7,8-tetrahydronaphthalene-2-ol;
cis-6-phenyl-5-(4-(2-pyrrolidin-1-yl-ethoxy)-phenyl)-5,6,7,8-tetrahydronaphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,4-tetrahydroisoquinoline;
cis-6-(4-hydroxyphenyl)-5-(4-(2-piperidin-1-yl-ethoxy)-phenyl)-5,6,7,8-tetrahydro-naphthalene-2-ol; or 1-(4′-pyrrolidinolethoxyphenyl)-2-phenyl-6-hydroxy-1,2,3,4-tetrahydroisoquinoline is in the range of 0.0001 to 100 mg/kg/day, preferably 0.001 to 10 mg/kg/day.

The compounds of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

Pharmaceutical compositions of the invention may contain 0.1%-95% of the compound(s) of this invention, preferably 1%-70%. In any event, the composition or formulation to be administered will contain a quantity of a compound(s) of this invention in an amount effective to treat the disease/condition of the subject being treated.

The present invention can also be administered using an injectable, flowable composition that provides sustained release at the local site of the injection by forming a biodegradable solid or gel depot, matrix or implant. An example of such an administration system is an EP₂ selective, receptor agonist compound in a slow-release biodegradable polymer based delivery system.

The polymer based delivery system contains EP₂ selective receptor agonist compound, and optionally any additional therapeutically useful compounds, dissolved or dispersed in biodegradable, thermoplastic polymer solution or dispersion in an organic solvent. Upon injection of the flowable composition, the organic solvent diffuses away from the injection site, causing the polymer to precipitate or gel; thereby entrapping the compound in a sustained-release depot. The compound is subsequently released by diffusion from, and erosion of, the polymeric matrix. The polymeric matrix slowly erodes by hydrolysis and eventually disappears from the site of administration. The molecular weight and concentration of the polymer can control the in vivo release of the compound as well as the degradation rate of the matrix.

The polymer based delivery system provides sustained release of an EP₂ selective receptor agonist compound in vivo for a sustained period of time with minimum or reduced burst in a patient in need thereof. A large burst of compound would result in poor local toleration due to local effects of the compound (e.g., irritation) and would minimize the amount of compound available for efficacy. The advantages this administration method is that it minimizes or reduces the initial burst, but still delivers compound at efficacious levels for a sustained period of time upon a single local injection.

The polymer system is prepared by contacting the flowable composition with a gelation medium to coagulate or gel the composition into a solid, microporous polymeric matrix or a gel polymeric matrix. The flowable composition contains a thermoplastic polymer or copolymer in combination with a suitable solvent. The polymers or copolymers, which form the body of the matrix, are substantially insoluble, preferably essentially completely insoluble, in water and body fluids. The insolubility of the matrix body enables it to function as a single site for the controlled release of the EP₂ selective receptor agonist compound. The polymers or copolymers also are biocompatible and biodegradable and/or bioerodible within the body of an animal, e.g., mammal. The biodegradation enables the patient to metabolize the polymer matrix so that it can be excreted by the patient without the need for further surgery to remove it. Because, the, flowable composition and polymer system are biocompatible, the insertion process and the presence of the polymer system within the body do not cause substantial tissue irritation or necrosis at the implant site. The composition of the present invention is administered as a flowable composition directly into body tissues.

Suitable thermoplastic polymers for incorporation into the solid matrix of the controlled release polymer system are solids, pharmaceutically compatible and biodegradable by cellular action and/or by the action of body fluids. Examples of appropriate thermoplastic polymers include polyesters of diols and dicarboxylic acids or of hydroxycarboxylic acids, such as polylactides, polyglycolides and copolymers thereof. More preferably the polymer is the copolymer, poly-lactic-co-glycolic acid (abbreviated PLGH), which upon hydrolysis, produces lactic and glycolic acid. The burst of release of this copolymer can be minimized further by the addition of polyethylene glycol (PEG) to form the PEG end-capped PLGH.

Preferred materials for use in the present invention are the polylactides, polyglycolides and copolymers thereof. These polymers can be used to advantage in the polymer system in part because they show excellent biocompatibility. They produce little, if any, tissue irritation, inflammation, necrosis or toxicity. In the presence of water, these polymers produce lactic and glycolic acid, respectively, which are readily, metabolized by the body. The polylactides can also incorporate glycolide monomer to enhance the resulting polymer's degradation. These polymers can also be used because they effectively control the rate of release of the EP₂ selective receptor agonist compound from the polymer system, and because they result in the local retention of the EP₂ selective receptor agonist compound at the site of the site of administration.

The solubility or miscibility of a thermoplastic polymer in the organic solvent of the composition will vary according to factors such as crystallinity, hydrophilicity, capacity for hydrogen bonding and molecular weight of the polymer. Consequently, the molecular weight and the concentration of the polymer in the solvent are adjusted to achieve desired miscibility, as well as a desired release rate for the incorporated EP₂ selective receptor agonist compound.

The flowable composition of thermoplastic polymer, solvent and the EP₂ selective receptor agonist compound is a stable flowable substance. A homogenous solution of the EP₂ selective receptor agonist compound in an organic solvent preferably results. The thermoplastic polymer is substantially soluble in the organic solvent. Upon placement of the flowable composition into the body, the solvent will dissipate and the polymer will solidify or gel to form the polymer system having the EP₂ selective receptor agonist compound within a solid or gel polymeric matrix.

It has been discovered that the molecular weight of the polymer used distinctly affects the rate of release of the EP₂ selective receptor agonist compound and the rate of degradation of the polymer from the site as long as the flowable composition has been used as an intermediate.

For certain preferred polymers for use in the present invention, the molecular weight of the polymer or copolymer is adjusted to be within a range of about 0.2 to about 0.4 inherent viscosity (I.V. in deciliters/g) for effective sustained release of the bone growth promoting compound. The typical rate of release of the incorporated bone growth promoting compound occurs at an I.V. of about 0.2 (about 8,000 to about 16,000 molecular weight) or about 0.3 (about 23,000 to about 45,000 molecular weight) but can vary depending on the particular components of the composition. For most systems, it is preferred to adjust the molecular weight of the polymer to about 0.2 I.V. for an effective sustained release of the EP₂ selective receptor agonist compound. The unit of measure for the molecular weight is daltons.

For a poly(DL-lactide) or a lactide-co-glycolide polymer system, the desired molecular weight range is about 0.2 to about 0.4 I.V., with an I.V. of about 0.2 being most preferred. The molecular weight of a polymer can be varied by any of a variety of methods. The choice of method is typically determined by the type of polymer composition. The preferred polymers for use are commercially available. Highly preferred thermoplastic polymers for use in the present invention are the following: PLGH copolymer with 1:1 ratio of lactic and glycolic acid with an inherent viscosity of about 0.2 dl/g (commercially available from Boehringer Ingelheim as Copolymer RESOMER® RG 502H) (about 12,000 molecular weight); PLGH copolymer with 1:1 ratio of lactic and glycolic acid with an inherent viscosity of about 0.3 dl/g (commercially available from Boehringer Ingelheim as Copolymer RESOMER® RG 503H)(about 37,000 molecular weight); PLGH copolymer with 1:1 ratio of lactic and glycolic acid with an inherent viscosity of about 0.4 dl/g (commercially available from Boehringer Ingelheim as Copolymer RESOMER®D RG 504H) (about 47,000 molecular weight); and polyethylene glycol (PEG) end-capped PLGH copolymer with 1:1 ratio of lactic and glycolic acid with an inherent viscosity of about 0.79 dl/g (commercially available from Boehringer Ingelheim as PLG-PEG) (about 52,000 molecular weight). By appropriate choice of the polymer molecular weight and viscosity, the rate and extent of release of the EP₂ selective receptor agonist compound from the polymer system can be varied from very fast to very slow. For example, according to the present invention, the release rate of (3-(((4-tert-butyl-benzyl)-(pyridine-3-sulfonyl)-amino)-methyl)-phenoxy)-acetic acid, sodium salt, can be slowed to produce substantially complete release of the compound within about seven days. With the use of a greater viscosity of polymer according to the present invention, the period of time can be increased to about fourteen days. The desired release rate of the EP₂ selective receptor agonist compound will depend on several factors, such as the species of animal being treated as well as the specific condition being treated.

The concentration of the polymer in the system can also be varied to adjust the release rate of the incorporated EP₂ selective receptor agonist compound. It has been discovered that the more dilute the polymer concentration, the more readily the EP₂ selective receptor agonist compound compound will be released. This effect can be used in combination with other methods to more effectively control the release of the incorporated EP₂ selective receptor agonist compound as desired. For example, by adjusting the concentration of the polymer and EP₂ selective receptor agonist compound, if desired, a wide range of release rates can be obtained.

The solvents used in the thermoplastic compositions of the present invention are preferably pharmaceutically acceptable, biocompatible and will dissipate into body fluid in situ such that they may be classed as having a solubility in water ranging from highly soluble to insoluble. Preferably, they cause relatively little, if any, tissue irritation or necrosis at the site of the injection and implantation. Preferably, the solvent may have at least a minimal degree of water solubility. When the organic solvent is water insoluble or is minimally soluble in water, the solvent will slowly disperse from the flowable polymeric composition. The result will be an implant that during the course of its life may contain a varying amount of residual solvent. Especially preferably, the organic solvent has a moderate to high degree of water solubility so that it will facilely disperse from the polymeric composition into the body fluids. Most preferably, the solvent disperses rapidly from the polymeric composition so as to quickly form a solid implant. Concomitant with the dispersion of solvent, the thermoplastic polymer coagulates or gels into the solid polymer system. Preferably, as the thermoplastic polymer coagulates, the solvent dispersion causes pore formation within the polymer system. As a result, the flowable composition containing thermoplastic polymer, solvent and EP₂ selective receptor agonist compound will form a porous solid polymer system. Also, when the solvent is slightly water soluble or is water insoluble, the solvent dispersion may result in the formation of a solid porous implant, or if some solvent remains with the implant, the result may be formation of a gel implant having few or no pores.

Suitable solvents include those liquid organic compounds meeting the foregoing criteria. The preferred solvent for use in the present invention is N-methyl-2-pyrrolidone (NMP) due, at least in part, to its solvating ability and its biocompatibility.

The solvents for the thermoplastic polymer flowable compositions are chosen for compatibility and appropriate solubility of the polymer and solvent. Lower molecular weight thermoplastic polymers will normally dissolve more readily in the solvents than high molecular weight polymers. As a result, the concentration of a thermoplastic polymer dissolved in the various solvents differs depending upon type of polymer and its molecular weight. Conversely, the higher molecular weight thermoplastic polymers will tend to coagulate, gel or solidify faster than the very low molecular weight thermoplastic polymers. Moreover, the higher molecular weight polymers tend to give higher solution viscosities than the low molecular weight materials. Thus, for advantageous injection efficiency, in addition to advantageous release rate, the molecular weight and the concentration of the polymer in the solvent are controlled.

Upon formation of the polymer system from the flowable composition, the EP₂ selective receptor agonist compound becomes incorporated into the polymer matrix. After insertion of the flowable composition to form in situ the polymer system, the EP₂ selective receptor agonist compound will be released from the matrix into the adjacent tissues or fluids by diffusion and polymer degradation mechanisms. Manipulation of these mechanisms also can influence the release of the EP₂ selective receptor agonist compound into the surroundings at a controlled rate. For example, the polymer matrix can be formulated to degrade after an effective and/or substantial amount of the EP₂ selective receptor agonist compound is released from the matrix. Thus, the release of the EP₂ selective receptor agonist compound from the matrix can be varied by, for example, the solubility of the EP₂ selective receptor agonist compound in water, the distribution of the bone growth promoting compound within the matrix, or the size, shape, porosity, solubility and biodegradability of the polymer matrix, among other factors. The release of the EP₂ selective receptor agonist compound from the matrix: is controlled relative to its inherent rate by varying the polymer molecular weight to provide a desired duration and rate of release.

For example, a preferred dosage form of the EP₂ selective receptor agonist compound, (3-(((4-tert-butyl-benzyl)-(pyridine-3-sulfonyl)-amino)-methyl)-phenoxy)-acetic acid, is a lyophile of the sodium salt to be reconstituted with a solution of PLGH in NMP before administration. The dosage form, consisting of the lyophilized compound in one syringe (syringe A) and a solution of PLGH in NMP in a second syringe (syringe B), is known as the A/B reconstitution system. The contents of both syringes are mixed together immediately prior to dose delivery at or near site. After reconstitution, the contents are transferred into a graduated dosing syringe for delivery. The administered dosage forms will be a solution and will result in the dispersion of the compound with PLGH in NMP at desired strengths of, for example, 5 and 50 mgA/ml (mgA/ml refers to the free acid equivalent of the sodium salt form of the compound). The dosage form is a parenteral (e.g., subcutaneous, intramuscular or intramedullary) sustained release injection for local administration. This compound in a slow-release polymer matrix (depot injection) is designed for administration at or near a site, and is not intended for intravenous administration. To provide adequate shelf-life stability for the dosage form, a two-syringe system (A/B), as described above, may be used, preferably with the sodium salt form of the compound. A uniphase formulation, preferably with the free acid form of the compound, is a preferred alternative formulation. Based on the compound and polymer stability, sterile filtration of the compound and irradiation of the polymer solution may be preferred for manufacturing a stable sterile product. In one embodiment, the dosage form can be manufactured and shipped as separate aluminum pouches containing syringes filled with the lyophile form of the compound in one pouch and the polymer solution in the other pouch. Delivery containers, systems and methods for the lyophilization of the bone growth promoting compounds of the present invention to prepare pharmaceutical compositions and kits are described in published International patent application WO 01/73363.

### EXAMPLE A

To obtain dosage form at strengths of 5 and 50 mgA/ml, the following combinations A) and B) of lyophile and polymer syringe, respectively, were used:
A) 5 mgA/ml (upon reconstitution) of (3-(((4-tert-butyl-benzyl)-(pyridine-3-sulfonyl)-amino)-methyl)-phenoxy)-acetic acid, sodium salt formulation:
Drug Syringe A contained 4 mgA of the sodium salt lyophile in 1.25 ml male syringe without graduations; and
Vehicle Syringe B contained 0.8 ml 50% RG502H/50% NMP solution in 1.25 ml female syringe without graduations.
B) 50 mgA/ml (upon reconstitution) of (3-(((4-tert-butyl-benzyl)-(pyridine-3-sulfonyl)-amino)-methyl)-phenoxy)-acetic acid, sodium salt formulation:
Drug Syringe A contained 40 mgA of the sodium salt lyophile in 1.25 ml male (fat) B-D syringe without graduations; and
Vehicle Syringe B contained 0.8 ml 50% RG502H/50% NMP solution in 1.25 ml female (thin) syringe without graduations.

MgA refers to free acid equivalent of the sodium salt form of the compound;
The percentages used in these examples are based on the weight of the indicated ingredients;
RG502H is a PLGH copolymer with 1:1 ratio of lactic and glycolic acid with inherent viscosity of 0.2 dl/gm, which is commercially available such as from Boehringer Ingelheim as Copolymer RESOMER® RG 502H.

### EXAMPLE 1

50% RG502H/50% NMP with 5 mgA/ml of sodium salt of (3-(((4-tert-butyl-benzyl)-(pyridine-3-sulfonyl)-amino)-methyl)-phenoxy)-acetic acid, mixed A/B (polymer solution autoclaved, compound lyophilized)

### EXAMPLE 2

50% RG502H/50% NMP with 10 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized)

### EXAMPLE 3

50% RG502H/50% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized)

### EXAMPLE 4

47% RG502H/3% PLG-PEG/50% NMP with 50 mgA/ml of sodium Salt of (3-(((4-tert-butyl-benzyl)-(pyridine-3-sulfonyl)-amino)-methyl)-phenoxy)-acetic acid, uniphase

### EXAMPLE 5

47% RG503H/3% PLG-PEG/50% NMP with 50 mgA/ml of sodium salt of (3-(((4-tert-butyl-benzyl)-(pyridine-3-sulfonyl)-amino)-methyl)-phenoxy)-acetic acid, uniphase

### EXAMPLE 6

45% RG504H/55% NMP with 50 mgA/ml of sodium salt of (3-(((4-tert-butyl-benzyl)-(pyridine-3-sulfonyl)-amino)-methyl)-phenoxy)-acetic acid, uniphase

### EXAMPLE 7

37% RG503H/63% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution autoclaved, compound lyophilized)

### EXAMPLE 8

37% RG503H/63% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized)

### EXAMPLE 9

50% RG502H/50% NMP with 5 mgA/ml of (3-(((4-tert-butyl-benzyl)-(pyridine-3-sulfonyl)-amino)-methyl)-phenoxy)-acetic acid, uniphase.

Further exemplification of the polymer matrix delivery system described above can be found in U.S. provisional patent application No. 60/337,255, filed Nov. 30, 2001.

In a preferred administration system, syringe A contains the lyophile of the sodium salt of (3-(((4-tert-butyl-benzyl)-(pyridine-3-sulfonyl)-amino)-methyl)-phenoxy)-acetic acid which in made to result in 4 mg per syrinige or 40 mg per syringe. Syringe B contains Resomer 502H, 50:50 Poly(D,L lactide-co-glycolide), (50,50 PLGH) and N-methyl-2-pyrroliddone (NMP). The dose using the above describe A/B syringe system can vary widely and is deterimed by the disese being treated among other factors. A preffered dose range includes 0.5 mgA up to about 100 mgA. In syringes containing 50 mgA/ml, preferred doses include 0.1 ml, 0.2 ml, 0.6 ml and 2 ml. In syringes contianing 5 mgA/ml, preferred doses include 0.1 ml, 0.2 ml, 0,3 ml, 0.6 and 2 ml.

Other methods of administration of an EP₂ selective receptor agonist include local administration by injection to a particular site or delivery by a catheter to a site. Additional examples can be found in U.S. provisional patent application No. 60/335,156, filed Nov. 30, 2001.

### General Experimental Procedures

In general the compounds of this invention can be made by processes which include processes known in the chemical arts, particularly in light of the description contained herein. Certain processes for the manufacture of the compounds of this invention are provided as further features of the invention and are illustrated by the following reaction schemes. Other processes are described in the experimental section.

Some substituents (e.g., carboxyl) may best be prepared through conversion of another functional group (e.g., carboxyl substituents may be prepared through conversion of, e.g., hydroxyl or carboxaldehyde) at a point later in the synthetic sequence.

Compounds of Formula I wherein B is nitrogen may be prepared using methods described in SCHEMES 1-5. These methods include (a) sequential alkylation of a sulfonamide or amide with two appropriate alkylating agents, generally alkyl halides or alkyl sulfonates; (b) alkylation of a sulfonamide or amide with an alkyl halide or alkyl sulfonate; or (c) reductive amination of an aldehyde followed by reaction with an acylating agent such as an acyl chloride, a chloroformate, an isocyanate or a chlorocarbonyl amide; or a sulfonylating agent such as a sulfonyl chloride. When performing sequential alkylation, one of the alkylating agents will contain a Q-Z portion, where the Z portion is suitably protected if necessary, and the other alkylating agent will contain a K-M portion, where any functional groups requiring protection are suitably protected. The order of the alkylation, i.e., whether the alkylating agent containing the Q-Z portion is added first or second, will depend upon the reactivity of the electrophilic side chain. When performing a reductive amination, the Q-Z portion may be attached to either, the amine reagent or the aldehyde reagent depending upon the ease of preparation of the reagent and the reactivity of the reagents in the reductive amination reaction. The reductive amination is followed by acylation or sulfonylation with an appropriate acylating agent or sulfonyl chloride and, if desired the product is hydrolysed. The starting materials, including amines, aldehydes and alkylating agents, are prepared using methods well known to those skilled in the art. Certain preferred methods for their preparation are described herein.

For example, compounds of Formula I wherein B is N are prepared of the methods set forth in SCHEMES 1 and 2 below. In general, the sequences involve sequential alkylation of an appropriate sulfonamide of Formula 1 or amide of Formula 1 with two appropriate alkyl halides or alkyl sulfonates. SCHEMES 1 and 2 differ only in the order of addition of the two alkylating agents. The alkylation order is typically chosen depending on the reactivity of the electrophilic side-chain. It is generally preferable to react the less reactive electrophilic side chain first. This reduces the amount of dialkylation which occurs in that first alkylation step, thereby resulting in a greater yield of monoalkylated material to be carried on to the next alkylation. In SCHEMES 1 and 2, one of the alkylating agents contains a carboxylic acid or a carboxylic acid isostere, suitably protected with an appropriate protecting group, if necessary. Further, in SCHEMES 1 and 2, the carboxylic acid precursor of Formula 3 is a carboxylic acid ester where R is a suitable carboxylic acid protecting group. Generally, the protecting group is either a straight chain lower alkyl, preferably methyl or ethyl, or a tert-butyl or phenyl group. Other acid isosteres can be employed by appropriately modifying SCHEMES 1 and 2 of methods well known to those skilled in the art (e.g., see SCHEME 6 which sets forth the preparation of a tetrazole). Typical alkylating agents are primary, secondary, benzylic or allylic halides and sulfonates and are preferably alkyl bromides or alkyl iodides.

The Formula 1 sulfonamide or amide is converted to its anion with a strong base such as sodium hydride, lithium diisopropylamide, lithium bis(trimethylsilyl)amide, potassium bis(trimethylsilyl)amide, potassium tert-butoxide, etc. in an aprotic solvent such as dimethylformamide, tetrahydrofuran or N,N-dimethylformamide/benzene at a temperature of about −78° C. to about 100° C. The resulting anion is alkylated with an appropriate alkyl halide of Formula 2 or 3 or an appropriate alkyl sulfonate of Formula 2 or 3, wherein X′ is the halide or sulfonate portion of the alkylating agent, at a temperature of about 0° C. to about 100° C. to yield the corresponding mono-alkylated compound of Formula 4 or 5. In some cases, varying amounts of a side-product resulting from dialkylation of the amide or sulfonamide are obtained and can be removed using chromatographic techniques, preferably by flash chromatography (W. C. Still, M. Kahn, A. Mitra, J. Org. Chem. 43, 2923, 1978). After the first alkylation is complete, the compound of Formula 4 or 5 is converted to an anion using a suitable base such as sodium hydride, lithium bis(trimethylsilyl)amide, lithium diisopropylamide, potassium bis(trimethylsilyl)amide, potassium tert-butoxide, or potassium carbonate in an aprotic solvent such as N,N-dimethylformamide, tetrahydrofuran, N,N-dimethylformamide/benzene, or acetone at a temperature of about −78° C. to about 100° C. Alkylation of the anion with an appropriate second alkyl halide of 0Formula 3 or 2 or alkyl sulfonate of Formula 3 or 2 provides the corresponding dialkylated compound of Formula 6. When R is methyl or ethyl, the ester of Formula 6 is hydrolyzed to the corresponding carboxylic acid of Formula I with a dilute aqueous basic solution. This hydrolysis is preferably carried out using sodium or potassium hydroxide in aqueous methanol or ethanol, lithium hydroxide in aqueous alcoholic solvent or aqueous tetrahydrofuran at a temperature of about 0° C. to about 80° C. Alternatively, the hydrolysis may be carried out by using methods well known to those skilled in the art, for example, methods described in “Protecting Groups in. Organic Synthesis,” Second Edition, T. W. Greene and P. G. M. Wuts, John Wiley and Sons, Inc., 1991.

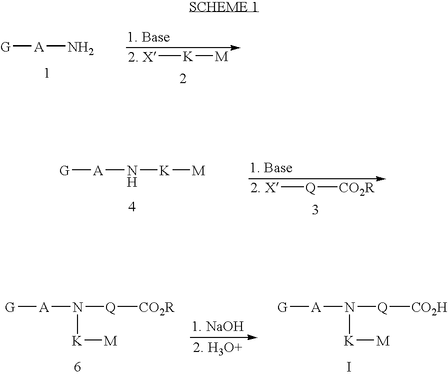

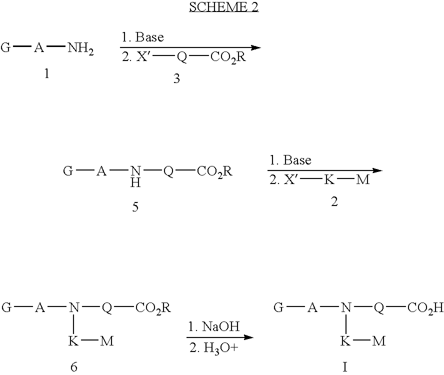

Compounds of Formula I wherein B is N are also prepared from amines as set forth in SCHEMES 3-4. Generally, the appropriate amine starting materials of Formulas 9 and 10 are commercially obtained or can be prepared using methods well known to those skilled in the art (see “The Chemistry of Amino, Nitroso and Nitro Compounds and their Derivatives,” Ed. S. Patai, J. Wiley, New York, 1982). For example, the amine starting materials are prepared from the corresponding nitriles of Formulas 7 or 8. Said nitriles are available from commercial sources or can be prepared using methods well known to those skilled in the art (see Rappaport, “The Chemistry of the Cyano Group,” Interscience, New York, 1970 or Patai and Rappaport, “The Chemistry of Functional Groups,” pt. 2, Wiley, New York, 1983). The nitrile of Formula 7 or 8 is reduced with a reducing agent such as borane-tetrahydrofuran complex, borane-methyl sulfide complex or lithium aluminum hydride in an aprotic solvent such as tetrahydrofuran or diethyl ether at a temperature of about −78° C. to about 60° C. Alternatively, the nitrile is hydrogenated under a hydrogen atmosphere typically at 0 to 50 psi in the presence of Raney nickel or a platinum or palladium catalyst in a protic solvent such as methanol or ethanol at a temperature of about 0° C. to about 50° C. It may be desired to add an equivalent of an acid, such as hydrogen chloride, to accomplish the reduction. The amine of Formula 9 or 10 thus obtained is converted to the sulfonamide of Formula 11 or 12 by sulfonylation with a sulfonyl chloride or said amine is converted to an amide of Formula 11 or 12 by acylation with an appropriate acyl chloride. Both the sulfonylation reactions and the acylation reactions are generally carried out in the presence of a weak base such as triethylamine, pyridine, or 4-methylmorpholine in an aprotic solvent such as methylene chloride or diethyl ether at a temperature of about −20° C. to about 50° C., Alternatively, coupling of amines of Formulas 9 or 10 with carboxylic acids are conveniently carried out in an inert solvent such as dichloromethane or N,N-dimethylformamide by a coupling reagent such as 1-(3-dimethylaminopropyl)-3-ethylcarbodiimide hydrochloride (EDC) or 1, 3-dicyclohexylcarbodiimide (DCC) in the presence of 1-hydroxybenzotriazole hydrate (HOBT) to generate compounds of Formulas 11 or 12. Where the amine is present as the hydrochloride or other salt, it is preferable to add one equivalent of a suitable base such as triethylamine to the reaction mixture. Alternatively, the coupling can be effected with a coupling reagent such as benzotriazol-1-yloxy-tris(dimethylamino)-phosphonium hexafluorophosphate (BOP) in an inert solvent such as methanol. Such coupling reactions are generally conducted at temperatures of about −30° C. to about 80° C., preferably 0° C. to about 25° C. For a discussion of other conditions used for coupling peptides see Houben-Weyl, Vol. XV, part 11, E. Wunsch, Ed., George Theime Verlag, 1974, Stuttgart. Alkylation and if desired, deprotection, of the Formula 11 or 12 compound as described in SCHEMES 1 and 2 affords the corresponding acid Formula 13 and 14 compound. The compounds of Formulas 11 and 12 are alkylated in a manner analogous to the alkylation of the compounds of Formulas 1, 4 and 5 of SCHEMES 1 and 2 hereinabove. The alkylated products are deprotected, if necessary, to afford the compounds of Formulas 13 and 14.

The amines of Formulas 9 and 10 are also prepared via reduction of an appropriate amide of Formulas 15 and 16. This reduction is achieved using reagents such as a borane-tetrahydrofuran complex, a borane-methyl sulfide complex, or diisobutyaluminum hydride in an aprotic solvent such as tetrahydrofuran or diethyl ether at a temperature of about −78° C. to about 60° C.

The amines of Formulas 9 and 10 are also obtained from the corresponding nitro precursors by reduction of the nitro group using reducing reagents such as zinc/HCl, hydrogenation in the presence of Raney nickel, palladium, or platinum catalysts, and other reagents as described by P. N. Rylander in “Hydrogenation Methods,” Academic Press, New York, 1985.

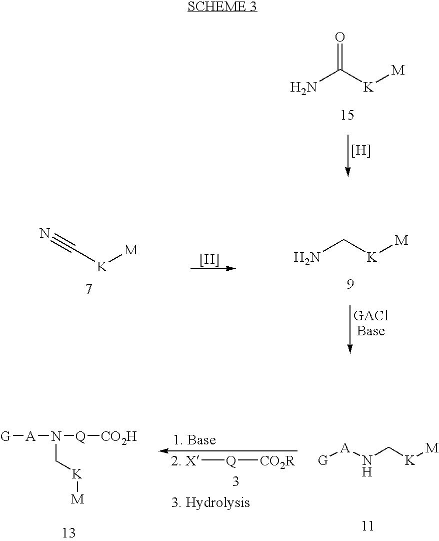

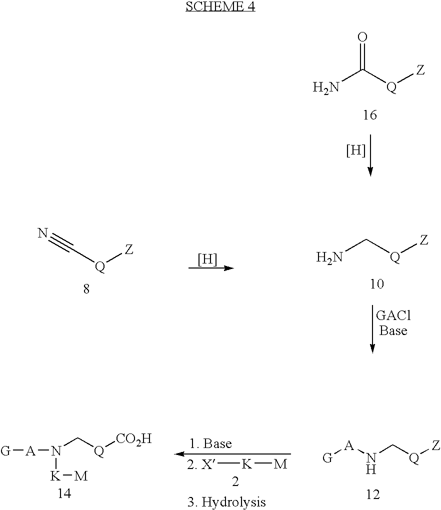

Amines and alkylating agents useful for the above syntheses are described and prepared as set forth in the section entitled PREPARATIONS below.

Alternatively, the compounds of Formula I wherein B is N are prepared by reductive amination of an aldehyde containing the appropriate suitably protected acidic functionality with an amine. This sequence is set forth in SCHEME 5. Alternatively, the amine may contain the appropriate suitably protected acidic functionality.

The reductive amination is typically carried out at a pH of between 6 and 8, using a reducing agent such as sodium cyanoborohydride or sodium triacetoxyborohydride. The reaction is normally performed in a protic solvent such as methanol or ethanol at temperatures of about −78° C. to about 40° C. (e.g., see A. Abdel-Magid, C. Maryanoff, K. Carson, Tetrahedron Lett. 39, 31, 5595-5598, 1990.) The reductive amination reaction may also be carried out using titanium isopropoxide and sodium cyanoborohydride (R. J. Mattson et al, J. Org. Chem. 1990, 55, 2552-4) or by preformation of the imine under dehydrating conditions followed by reduction. The resulting amine of Formulas 42 and 42A, is transformed to the desired amide or sulfonamide by coupling with an acid chloride, sulfonyl chloride, or carboxylic acid as set forth in SCHEMES 3 and 4. If desired, the amine intermediate of Formulas 42 or 42A may be converted to a urethane by treatment with a chloroformate or to a tetrasubstituted urea by treatment with a chlorocarbonyl amide. These reactions are performed in the presence of a weak base such as triethylamine, pyridine, or 4-methylrorpholine in an aprotic solvent such as methylene chloride or diethyl ether at a temperature of about −20° C. to about 50° C. Conversion of the amine of Formulas 42 or 42A to a trisubstituted urea is accomplished by treatment with an isocyanate in an aprotic solvent such as methylene chloride or diethyl ether at temperatures ranging between −20° C. and 50° C. (for example, see SCHEME 5A). In cases where the amine is present as the hydrochloride salt, it is preferable to add an equivalent of a suitable base such as trieihylamine to the reaction. If desired, hydrolysis of the resulting sulfonamide or amide provides the corresponding acid.

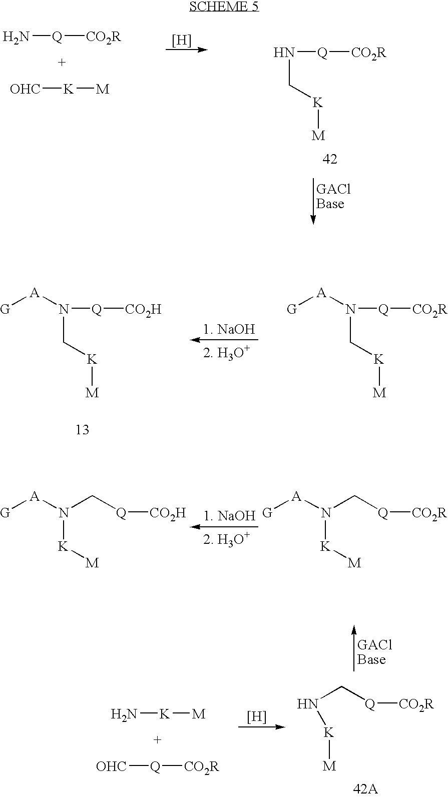

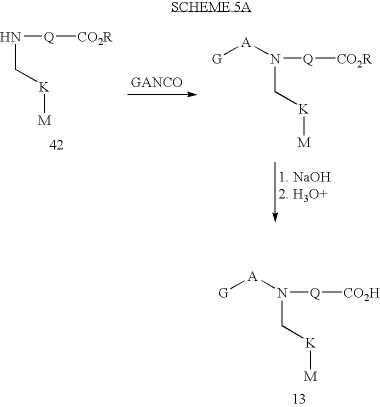

Aldehydes useful in the above SCHEME 5 are described and prepared as set forth in the section entitled PREPARATIONS below.

Compounds of Formula I where B is N and Z is tetrazolyl are prepared as set forth in SCHEME 6. A sulfonamide or amide of Formula 4 is alkylated with the appropriate alkyl halide or sulfonate (wherein X′ is halide or sulfonate), preferably a primary, secondary, benzylic, or allylic alkyl bromide, iodide, or sulfonate, which contains a nitrile to provide a nitrile of Formula 59. This alkylation is achieved by treatment of the sulfonamide or amide of Formula 59 with a base such as sodium hydride, lithium bis(trimethylsilyl)amide, potassium bis(trimethylsilyl)amide, potassium tert-butoxide, or potassium carbonate in an aprotic solvent such as dimethylformamide, dimethylformamide/benzene, or acetone followed by reaction of the resulting anion with a suitable alkylating agent. Alkylation occurs at a temperature of about −78° C. to about 100° C. A preferred method for converting the resulting nitrile of Formula 59 to the tetrazole of Formula 60 is treatment of the alkylated nitrile with dibutyltin oxide and trimethylsilylazide, in refluxing toluene (S. J. Wittenberger and B. G. Donner, J. Org. Chem. 1993, 58, 4139-4141, 1993). For a review of alternative preparations of tetrazoles see R. N. Butler, Tetrazoles, In Comprehensive Heterocyclic Chemistry; Potts, K. T. Ed.; Pergamon Press: Oxford, 1984, Vol. 5, pp 791-838.

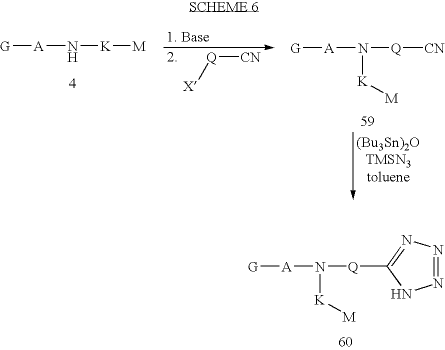

Alternatively, certain compounds of Formula I wherein B is N are prepared as set forth in SCHEME 7. Thus, esters of Formula 46 are prepared using the procedures described above in SCHEMES 1 and 2. Subsequent Heck coupling of this intermediate to an arylhalide (preferably an aryl bromide or aryl iodide), an aryl triflate, or a ring system which contains a vinyl bromide, iodide, or triflate is accomplished with a palladium catalyst, such as palladium acetate or tetrakis(triphenylphosphine)palladium(0) in the presence of a trialkylamine, such as triethylamine. In some cases, an additive such as a triarylphosphine or triarylarsene may be added to the reaction. The reaction is typically performed in an aprotic solvent such as dimethylformamide or acetonitrile at a temperature of about 0° C. to about 150° C. (see R. F. Heck in Comp. Org. Syn., Vol. 4, Ch. 4.3, p. 833 or Daves and Hallberg, Chem. Rev. 1989, 89, 1433). If desired, the compound of Formula 47 can be hydrolyzed to the corresponding acid. Alternatively, the compound of Formula 47 can be hydrogenated and, if desired, further hydrolyzed to the corresponding acid of Formula 49. Hydrogenation is preferably achieved under a hydrogen atmosphere typically at 0 to 50 psi in the presence of a palladium or platinum catalyst in an alcoholic solvent such as ethanol or methanol at a temperature of about 0° C. to about 50° C. In cases where M represents a partially saturated ring system, hydrogenation will generate a fully saturated ring system.

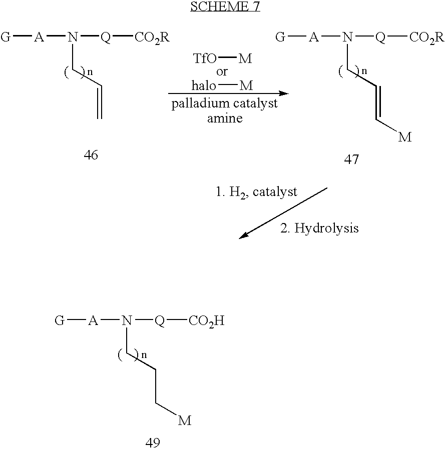

Alternatively, certain compounds of Formula I wherein B is N are prepared as described in SCHEME 8. Compounds of Formula 51 are prepared as described in SCHEMES 1 and 2 by alkylation of compounds of Formula 5 with an electrophile of Formula 2 which contains the appropriate functionality on the M ring., At least one of the substituents on the M ring must be suitable for subsequent conversion to an aldehyde. For example, electrophiles of Formula 2 containing a protected alcohol on the M ring may be alkylated and then deprotected and oxidized to the aldehyde, using reagents well known to those skilled in the art, to generate compounds of Formula 51. An alternative method is to alkylate with an electrophile of Formula 2 where M contains a vinyl group. After alkylation, oxidative cleavage of the double bond provides the desired aldehyde of Formula 51. The oxidative cleavage is accomplished by transforming the double bond to the 1,2-diol with catalytic osmium tetroxide and N-methylmorpholine followed by oxidative cleavage to the aldehyde using sodium periodate. Alternatively, oxidative cleavage via ozonolysis followed by reduction using reagents such as methyl sulfide, triphenylphosphine, zinc/acetic acid, or thiourea, generates the desired aldehyde of Formula 51. Addition of LMetal where LMetal is any organometallic reagent such as an organolithium or a Grignard reagent in an aprotic solvent such as diethyl ether or tetrahydrofuran at a temperature of about −78° C. to about 80° C., followed by hydrolysis of the ester as described above, provides the desired compound of Formula 50.

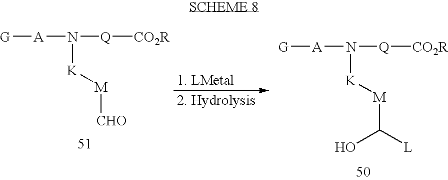

Alternatively, certain compounds of Formula I wherein B is N are prepared as described in SCHEME 9. The appropriate sulfonamide or amide of Formula 5 is alkylated using the conditions described in SCHEMES 1 and 2. The alkylating agent is an electrophile which contains an aromatic bromide or iodide or a ring system which contains a vinyl bromide or iodide (Ar¹) to provide compounds of Formula 53. Suzuki-type coupling of the compound of Formula 53 thus obtained with an aryl boronic acid (Ar²) provides Formula 53a compounds. For a review of the Suzuki reaction see A. R. Martinand Y. Yang in Acta Chem. Scand. 1993, 47, 221. The coupling reaction is achieved using about two equivalents of a base, such as sodium carbonate, potassium carbonate, sodium hydroxide, thallium hydroxide or potassium phosphate, in the presence of a palladium catalyst, such as tetrakis(triphenylphosphine)palladium(0), palladium acetate, palladium chloride, tris(dibenzylideneacetone)dipalladiurn(0) or [1,4-bis(diphenylphosphine)butane]palladium(0). The reaction may be run in an aqueous alcoholic solvent such as methanol or ethanol; or in other aqueous solvents such as aqueous tetrahydrofuran, aqueous acetone, aqueous glycol dimethyl ether, or aqueous benzene at temperatures ranging from about 0° C. to about 120° C. When Ar¹ is a partially saturated ring, reduction of the ring to provide a saturated ring system may, if desired, be performed at this point. This transformation is achieved by hydrogenating the partially saturated ring in the presence of a catalyst such as palladium or platinum in an alcoholic solvent (ethanol or methanol) and/or ethyl acetate. Ester hydrolysis of compounds of Formulas 53 or 53a, if desired, provides the corresponding acid. The resulting acids may contain functional groups on either of the ring systems (Ar¹ or Ar²) which can be modified using methods well known to those skilled in the art. Examples of such modifications are shown in SCHEME 10.

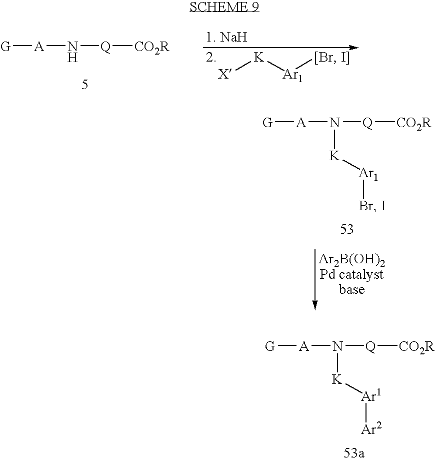

Compounds of Formula 54 which contain an aldehyde functional group are prepared using methods described in SCHEMES 8 and 9. Of SCHEME 10, treatment of a compound of Formula 54 with an appropriate organometallic reagent (LMetal), such as an organolithium or Grignard reagent, in an aprotic solvent such as diethyl ether or tetrahydrofuran at a temperature of about −78° C. to about 80° C., followed by hydrolysis of the ester, provides compounds of Formula 56. Alternatively, reduction of the aldehyde followed by hydrolysis provides Formula 55 compounds.

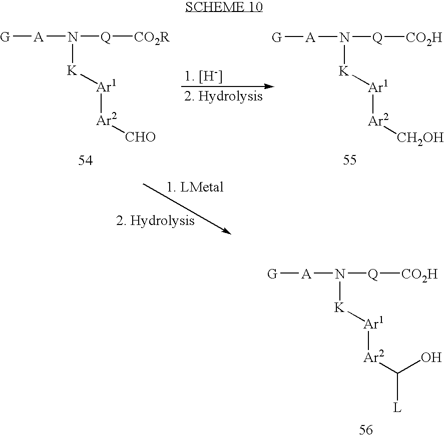

Alternatively, certain compounds of Formula I wherein B is N are prepared as described in SCHEME 11. The starting alcohols of Formula 58 are prepared of methods well known to persons skilled in the art, for example, by using methods described in SCHEMES 1 and 2. It will be recognized by a person of ordinary skill in the art that protecting groups may be required in the synthesis of certain of these alcohols. Intermediate 58 is coupled with a variety of aryl alcohols (M is as defined above) using Mitsonobu coupling conditions (for a review see O. Mitsonobu, Synthesis, 1, 1981). Typically the coupling is achieved by addition of a coupling agent such as triphenylphosphine and diethyl azodicarboxylate or diisopropyl azodicarboxylate in an inert solvent such as methylene chloride or tetrahydrofuran at a temperature of about 0° C. to about 80° C. If desired, subsequent hydrolysis yields the corresponding acid.

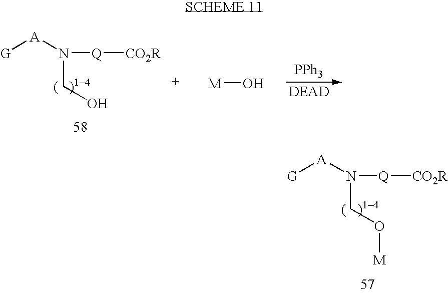

Alternatively, certain compounds of Formula I wherein B is N are prepared as described in SCHEME 12. A compound of Formula 102 is added to a compound of Formula 105 (wherein X is as defined above for the compound of Formula I) in the presence of a Lewis acid such as titanium tetrachloride or a mineral acid such as hydrochloric acid. If desired the ester of Formula 106 can be converted to the corresponding acid by hydrolysis or deprotection.

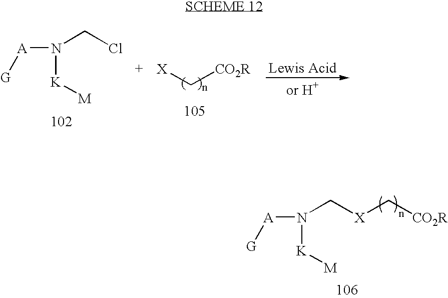

Alternatively, certain compounds of Formula I wherein B is N are prepared as described in SCHEME 13. Chloromethyl compounds of Formula 104 are treated with the appropriate substituted aromatic ring system, M, such as 4-ethoxybenzene or thiophene in the presence of a Lewis acid such as titanium tetrachloride or a mineral acid such as hydrochloric acid in an aprotic solvent such as chloroform at a temperature of about 0° C. to about 80° C. to yield compounds of Formula 107 which may subsequently be hydrolyzed or deprotected as described above to yield the corresponding carboxylic acids. Alternatively, chloromethyl compounds of Formula 104 can be treated with a Lewis acid such as titanium tetrachloride and an appropriately substituted vinyl silane in an aprotic solvent such as methylene chloride at a temperature of about 50° C. to about 50° C. to give compounds of Formula 108. If desired, the compounds of Formula 108 may subsequently be hydrolyzed or deprotected as described above to yield the corresponding acid. If desired, reduction of the double bond can be accomplished using conditions described in SCHEME 7.

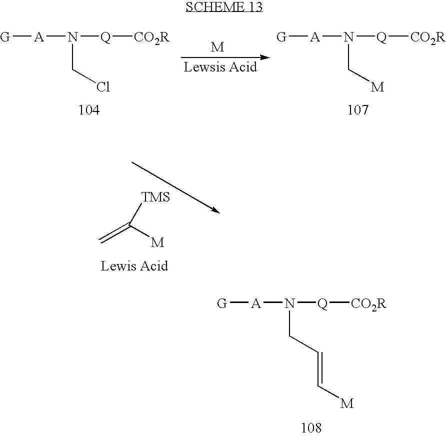

Alternatively, certain compounds of Formula I wherein B is N are prepared as described in SCHEME 14. Chloromethyl compounds of Formula 104 are treated with a Lewis acid such as titanium tetrachloride and an appropriately substituted allyl silane in an aprotic solvent such as chloroform at a temperature of about 0° C. to about 80° C. to give compounds of Formula 109 which may subsequently be hydrolyzed or deprotected as described above to afford the corresponding carboxylic acids.

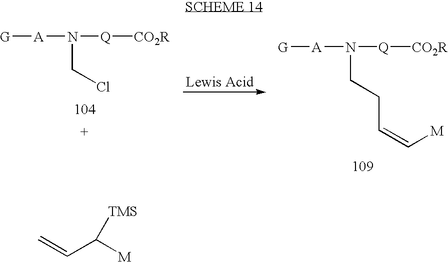

Alternatively, certain compounds of Formula I wherein B is N are prepared as described in SCHEME 15. Chloromethyl compounds of Formula 104 are treated with a sulfinic acid of Formula III in the presence of a base such as triethylamine in an aprotic solvent such as chloroform at a temperature of about −30° C. to about 50° C. to give compounds of Formula 112 which may subsequently be hydrolyzed or deprotected as described above to yield the corresponding acid.

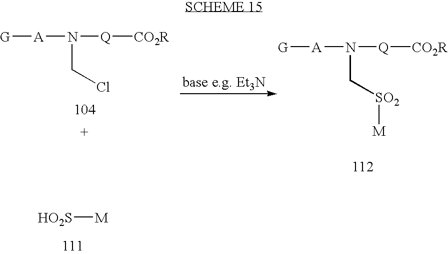

Formula I compounds wherein B is C(H) and Q, G, M and K are as described above in the Summary of the Invention can be prepared of SCHEME 16. Formula 0.113 beta-ketoesters are alkylated sequentially with Formula 114 compounds to form Formula 115 compounds followed by alkylation with Formula 116 compounds to give Formula 117 compounds (J. Med. Chem. 26, 1993, p 335-41). Alkylations can be carried out in a suitable solvent such as DMF, THF, ether, or benzene using an appropriate base such as sodium hydride, LDA, or potassium carbonate at a temperature of about −78° C. to about 80° C. The resulting Formula 117 disubstituted keto esters are hydrolyzed and decarboxylated to give the corresponding Formula 118 compound by using an aqueous base such as sodium hydroxide to hydrolyze the ester, followed by an acidic quench such as with aqueous hydrochloric acid to effect decarboxylation.

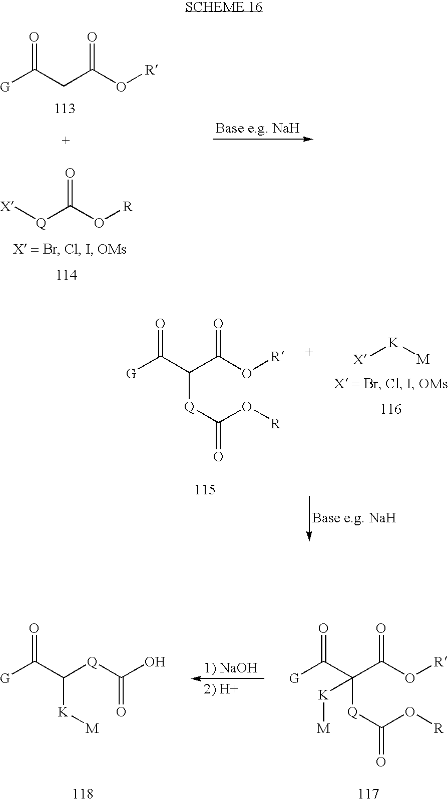

Alternatively, Formula I compounds wherein B is C(H) and G, Q, M and K are as described above in the Summary of the Invention may be prepared of SCHEME 17. Sequential alkylation of a malonate derivative of Formula 119 provides the Formula 121 dialkylated compound. Deprotection of the ester group by treatment with a strong acid such as TFA or HCl in ethanol at a temperature of about −20° C. to about 50° C. leads to the Formula 122 decarboxylated product. Conversion of the acid to an acid chloride using thionyl chloride or oxalyl chloride in an aprotic solvent at a temperature of about −78° C. to about 50° C. or to a Weinreb amide using methoxymethyl amine in the presence of a suitable coupling agent such as DCC or DEC in an aprotic solvent at a temperature of about −30° C. to about 50° C. provides Formula 123 compounds. Formula 123 compounds are suitable substrates for addition of various organometallic species, e.g., Grignard reagents and organo-cadmium reagents which, after hydrolysis of the terminal ester, provide the keto-acid compounds of Formula 118.

Alternatively, Formula 118 compounds can be prepared using methods described previously in Schemes 7-11 where one or both of the side chains are further functionalized after attachment to the alkanoyl fragment.

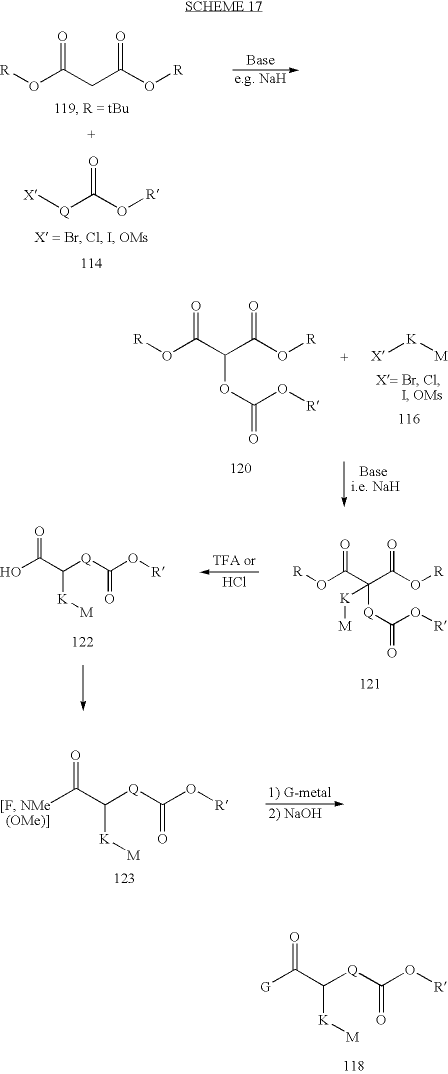

### Preparations

Amines, Amides and Sulfonamides

Certain amides or sulfonamides described by Formulas 21, 22, and 23 wherein W and Z are as described above in the Summary of the Invention and X and M are aromatic or saturated ring systems are prepared as set forth in SCHEME 18. Alkynyl amides or sulfonamides of Formulas 25, 26 and 27 are prepared by coupling an alkynyl sulfonamide or alkynyl amide of Formula 24 to an aromatic or vinyl halide, preferably an aromatic or vinyl bromide or iodide wherein W and Z are as defined above and where X and M represent an aromatic ring or a partially saturated ring system. The coupling is typically accomplished in the presence of copper iodide, a palladium catalyst, such as palladium chloride, bis(triphenylphosphine)palladium dichloride, or tetrakis(triphenylphosphine)palladium(0), and an amine such as triethylamine, diisopropylamine, or butylamine in an aprotic solvent such as acetonitrile at a temperature of about 0° C. to about 100° C. The alkynes of Formulas 25, 26 and 27 are converted to the corresponding alkanes of Formulas 21, 22 or 23 via hydrogenation in the presence of a palladium or platinum catalyst in a solvent such as methanol, ethanol, and/or ethyl acetate at a temperature of about 0° C. to about 50° C. In the case where M represents a partially saturated ring system, hydrogenation will convert M to a fully saturated ring system. Alternatively, the alkynes are converted to cis-alkenes using the Lindlar catalyst (Pd—CaCO₃—PbO) or other suitable catalyst. Alkylation and deprotection as described in SCHEMES 1 and 2 affords the corresponding compounds of Formula I.

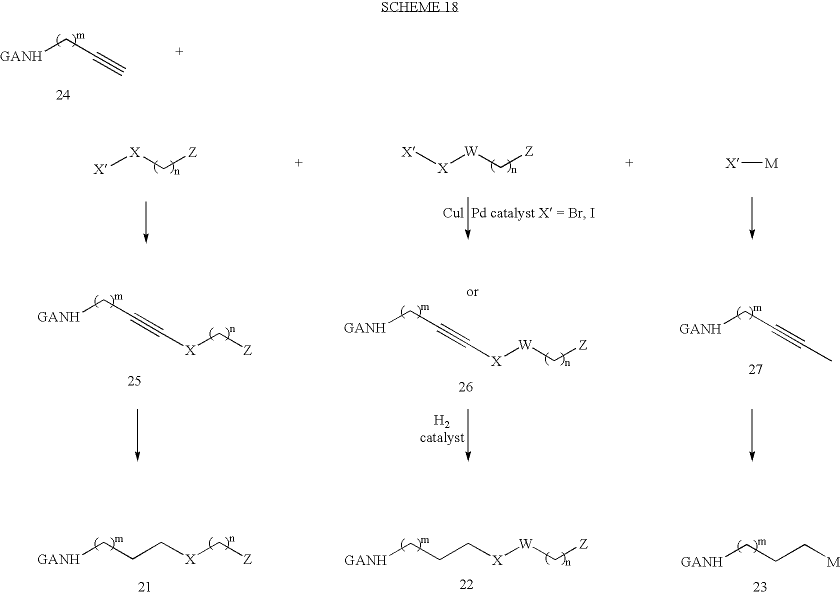

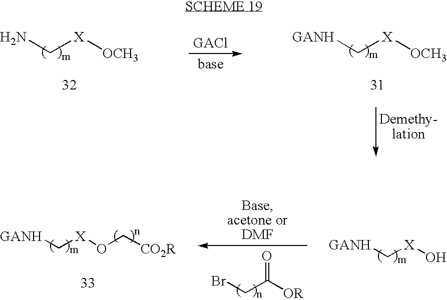

Compounds of Formula 33 are prepared from a suitable amine of Formula 32 (e.g., methoxyarylalkylamine). Amines of Formula 32 are commercially available or are prepared by methods well known to those skilled in the art (for example, see SCHEME 4). Amines of Formula 32 are converted to sulfonamides or amides of Formula 31 using methods, for example, described in SCHEME 3 and 4. The resulting aromatic methyl ether of Formula 31 is deprotected with reagents such as boron tribromide, pyridinium hydrochloride, hydrogen bromide/acetic acid, or other reagents as described in Protecting Groups in Organic Synthesis, Second Edition, T. W. Greene and P. G. M. Wuts, John Wiley and Sons, Inc., 1991. Alkylation with a bromoalkylester using a mild base such as potassium carbonate in an aprotic solvent such as dimethylformamide or acetone at a temperature of about 0° C. to about 100° C. generates an amide or sulfonamide of Formula 33.

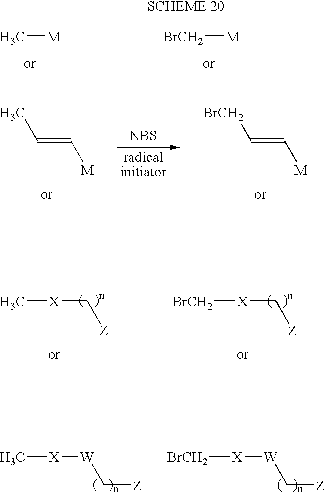

Alkylating Agents

Numerous methods exist for the synthesis of the desired alkylating agents used in the above procedures and are known to those skilled in the art (see “The Chemistry of the Carbon-Halogen Bond,” Ed. S. Patai, J. Wiley, New York, 1973 and “The Chemistry of Halides, Pseudo-Halides, and Azides,” Eds. S. Patai and Z. Rappaport, J. Wiley, New York, 1983). Some examples are shown in SCHEMES 20-24. As shown in SCHEME 20, tolyl or allylic substrates can be converted via halogenation to benzylic or allylic bromides wherein M, X, W and Z are as described above in the Summary of the Invention. This reaction is typically performed with N-bromosuccinimide (NBS) in the presence of a radical initiator such as 2,2′-azobisisobutyronitrile (AIBN) or a peroxide, preferably benzoyl peroxide. Alternatively, the reaction can be initiated with light. The reaction is performed in an inert solvent such as carbon tetrachloride or chloroform at a temperature of about 50° C. to about 100° C.

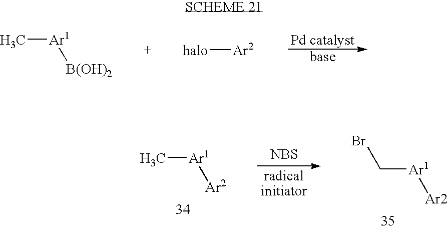

SCHEME 21 sets forth the synthesis of alkylating agents useful for preparing compounds of Formula I where M represents a biaryl or aryl cyclic group: Suzuki-type coupling of an aryl iodide or bromide or a ring system containing a vinyl bromide or iodide (Ar²) with a methylaryl boronic acid (Ar¹) using the conditions described in SCHEME 9 provides compounds of Formula 34. Where a vinyl bromide or iodide is used, compounds of Formula 34 can be reduced to generate a fully saturated ring. The reduction is accomplished by hydrogenation in the presence of palladium or platinum catalysts typically in protic solvents such as methanol or ethanol; or in tetrahydrofuran or ethyl acetate. Halogenation of the methyl group using reagents and conditions as described in SCHEME 20 provides alkylating agents of Formula 35.

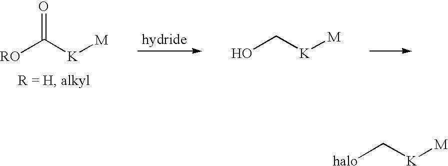

Another common method for accessing alkyl, halides is by halogenation of an alcohol or an alcohol derivative. Alcohols are obtained from commercial sources or can be prepared using methods well known to those skilled in the art. For example, SCHEME 22 sets forth the reduction of a carboxylic acid or ester to the corresponding alcohol using reagents such as, but not limited to, sodium borohydride, lithium aluminum hydride, borane-tetrahydrofuran complex, borane-methyl sulfide complex, etc. The corresponding alkyl chlorides are typically prepared from the alcohols with reagents such as hydrogen chloride, thionyl chloride, phosphorous pentachloride, phosphorous oxychloride, or triphenylphosphine/carbon tetrachloride. For the preparation of alkyl bromides, the alcohol is commonly treated with reagents such as hydrogen bromide, phosphorous tribromide, triphenylphosphine/bromine, or carbonyldiimidazole/allyl bromide (Kamijo, T., Harada, H., Iizuka, K. Chem. Pharm. Bull. 1983, 38, 4189). To access alkyl iodides, an appropriate alcohol is typically reacted with reagents such as triphenylphosphine/iodine/imidazole or hydogen iodide. Alternatively, alkyl chlorides can be converted to the more reactive alkyl bromides or alkyl iodides by reaction with an inorganic salt such as sodium bromide, lithium bromide, sodium iodide, or potassium iodide in solvents such as acetone or methyl ethyl ketone. Alkyl sulfonates can also be used as electrophiles or can be converted to alkyl halides. Alkyl sulfonates are prepared from the corresponding alcohol using a mild base such as triethylamine or pyridine and a sulfonyl chloride in an inert solvent such as methylene chloride or diethyl ether. If desired, conversion to the halide is accomplished by treating the alkyl sulfonate with an inorganic halide (sodium iodide, sodium bromide, potassium iodide, potassium bromide, lithium chloride, lithium bromide, etc) or a tetrabutylammonium halide.

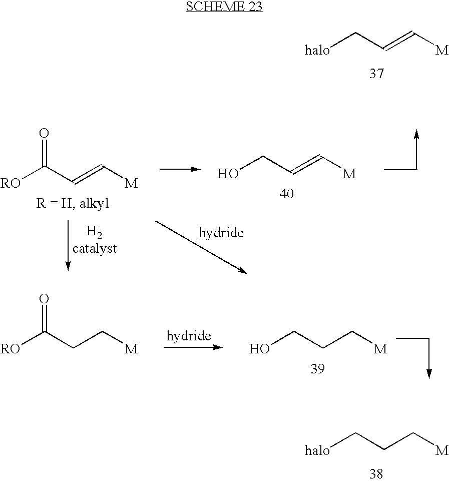

Cinnamic acids or esters are commonly available from commercial sources and can by converted to alkylating agents of Formulas 37 or 38 as follows (see SCHEME 23). The cinnamic acid or ester derivatives are reduced by hydrogenation in the presence of palladium or platinum catalysts typically in protic solvents (e.g., methanol or ethanol), tetrahydrofuran, or ethyl acetate. Reduction and conversion to the alkyl halide or sulfonate as described in SCHEME 22 provides the compounds of Formula 38. Where appropriate, the cinnamic acids or esters are converted directly to the alcohols of Formula 39 by treat those cinnamic acids or esters with reagents such as lithium aluminum hydride in an inert solvent such as tetrahydrofuran and diethyl ether. Alternatively, the cinnamic acid or ester can be reduced to an allylic alcohol of Formula 40 using reagents such as lithium aluminum hydride/aluminum chloride, diisobutylaluminum hydride or lithium borohydride. Conversion to the allylic halide or sulfonate as described in SCHEME 22 provides the compounds of Formula 37.

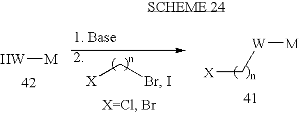

The preparation of alkylating agents of Formula 41 wherein W and M are as described above in the Summary of the Invention is set forth in SCHEME 24. Compounds of Formula 42 can be alkylated with a variety of bases. The choice of base is dependent on the nature of W and M. Some preferred bases include, but are not limited to, sodium hydroxide, sodium hydride, lithium diisopropylamide, lithium bis(trimethylsilyl)amide, potassium bis(trimethylsilyl)amide and potassium tert-butoxide. Treatment of the resulting anion with one of a variety of dialkylhalides generates the desired alkylating agents of Formula 41. For the preparation of compounds where W is an oxygen and M is an aromatic ring, it is preferred to form the alkoxide anion with sodium hydroxide followed by addition of a dihaloalkane, e.g. dibromoalkane. The reaction is normally performed in water at about 75° C. to about 125° C.

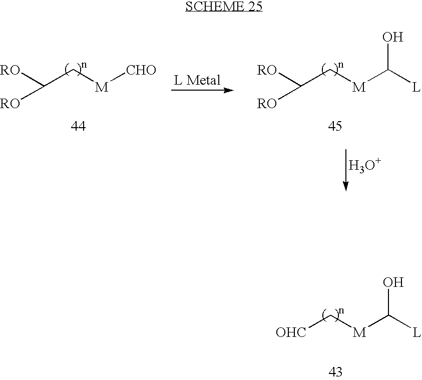

Aldehydes useful for the method described in SCHEME 5 are available from commercial sources or can be prepared from available intermediates using methods well known to those skilled in the art (for a general reference see “The Chemistry of the Carbonyl Group,” Ed. S. Patai, Interscience, New York (1966-70)). SCHEME 25 demonstrates an exemplary method used to prepare Formula 43 hydroxy aldehydes where M in SCHEME 5 contains a hydroxy substituted alkyl group. Treatment of a dialdehyde, where one of the aldehydes is protected as an acetal of Formula 44 wherein the OR groups are conventional substituents used in an acetal protecting group, with an organometallic reagent (LMetal), preferably an organolithium or Grignard reagent, in an inert solvent such as tetrahydrofuran or diethyl ether, provides compounds of Formula 45. Subsequent acetal hydrolysis under mildly acidic conditions, e.g., dilute hydrogen chloride, Amberlyst-15® resin, silica gel, or other reagents as described in “Protecting Groups in Organic Synthesis,” Second Edition, T. W. Greene and P. G. M. Wuts, John Wiley and Sons, Inc., 1991 provides the desired hydroxy aldehydes of Formula 43.

Aldehydes useful for the method described in SCHEME 5 may be prepared using the methods described in SCHEMES 26-28. For example, as shown in SCHEME 26, an aryl boronic acid which contains an aldehyde can be coupled to an aryl bromide, aryl iodide, or a ring system which contains a vinyl bromide or iodide using the Suzuki protocol described for SCHEME 9 to provide aldehydes of Formula 60.

SCHEME 27 describes the preparation of aldehydes of Formula 62 which contain a suitably protected acid moiety and can be used for the preparation of compounds of Formula 42A described in SCHEME 5. Selective reduction of nitrites (see SCHEMES 3-4 for preparations) of Formula 61, provides aldehydes of Formula 62. A preferred method for this reduction involves heating the nitrile with aluminum-nickel (Raney) alloy in the presence of an acid such as formic acid. Aldehydes of Formula 64 useful for the preparation of compounds of Formula 42 (SCHEME 5) may be prepared from starting nitrites of Formula 63 by treatment with a variety of reducing agents such as diisobutylaluminum hydride, tin (II) chloride/hydrogen chloride, or lithium triethoxyalanate.

A method for the preparation of proprionaldehydes of Formula 65 is described in SCHEME 28 and follows the procedures described by Kang (J. Org. Chem. 1996, 61, 2604) and by Jeffery (J. Chem. Soc. Chem. Commun. 1984, 19, 1287). An aryl iodide or bromide is coupled to allyl alcohol in the presence of a suitable palladium catalyst, preferably palladium acetate. The reaction is performed in a suitable polar, aprotic solvent, preferably dimethylformamide, with addition of a base, such as sodium bicarbonate, and an ammonium salt, such as tetrabutylammonium chloride and provides proprionaldehydes of Formula 65.

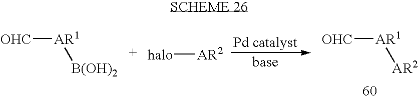

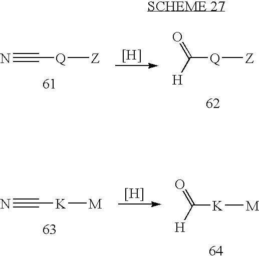

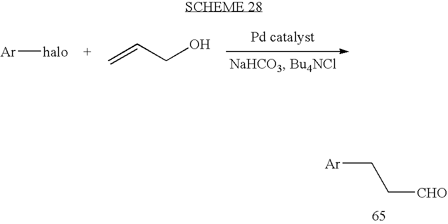

Chloromethyl Intermediates

Intermediate chloromethyl compounds can be prepared as described in SCHEMES 29 and 30. In general, the appropriate Formula 66 or 68 sulfonamide or carboxamide is treated with a formaldehyde equivalent such as paraformaldehyde in an inert organic solvent such as methylene chloride or chloroform with a suitable catalyst such as HCl, zinc chloride or trimethylsilyl chloride at temperatures ranging from about 0° C. to about 60° C. to give the Formula 67 and 69 chloromethyl derivatives, respectively.

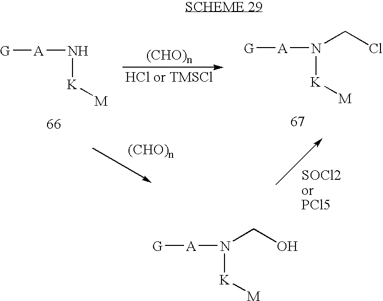

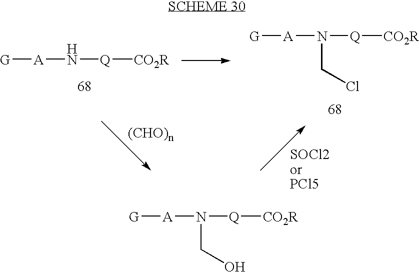

SCHEME 31 sets forth the synthesis of biaryl aldehydes of Formula 60. Fluorobenzonitriles of Formula 70 are heated with a nucleophilic group such as a pyrrazole or imidazole in a suitable solvent, preferably DMF to effect displacement of the fluoro group yielding intermediates of Formula 71. The desired biaryl aldehydes of Formula 60 are obtained by reduction of the nitrile via hydrogenation with Raney alloy in formic acid, or by reduction with a hydride source such as diisobutylaluminum hydride.

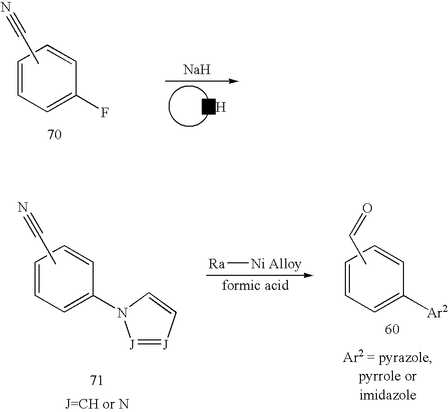

The examples and synthetic procedures set forth in this application are intended to illustrate particular embodiments of the invention and are not intended to limit the scope of the specification or claim in any manner. All documents cited in this application are hereby incorporated by reference.

### EXAMPLE 1

### 7-((4-Butyl-benzyl)-(pyridine-3-sulfonyl)-amino)-heptanoic acid

### Step A: Reductive Amination

7-(4-Butyl-benzylamino)-heptanoic acid methyl ester. A solution of 7-amino-heptanoic acid methyl ester hydrochloride, prepared of Preparation 1, (1.12 g, 5.9 mmol), 4-butyl-benzaldehyde (0.915 g, 5.65 mmol), and triethylamine (0.83 mL, 5.98 mmol) in 20 mL MeOH was stirred at room temperature for 3 hours. After cooling to 0° C., NaBH₄ (0.342 g, 9.04 mmol) was added and the reaction was stirred for 15 minutes at room temperature. The mixture was quenched with 1:1 NaHCO₃:H₂O and the MeOH was removed in vacuo. The resulting residue was diluted with CH₂Cl₂ and the organic solution was washed with water and brine, dried over MgSO₄, filtered, and concentrated in vacuo to afford the title compound of Step A (1.4 g). ¹H NMR (400 MHz, CDCl₃) δ 7.08-7.38 (m, 4H), 3.62 (s, 2H), 3.29 (s, 3H), 2.52-2.66 (m, 4H), 2.25 (t, 2H), 1.53-1.63 (m, 6H), 1.25-1.40 (m, 6H), 0.85 (t, 3H); MS 306 (M+1).

### Step B: Amide Formation

7-((4-Butyl-benzyl)-(pyridine-3-sulfonyl)-amino)-heptanoic acid methyl ester. A solution of 7-(4-butyl-benzylamino)-heptanoic acid methyl ester prepared of Example 1, Step A (0.10 g, 0.33 mmol); N,N-diisopropylethylamine (0.85 g 0.66 mmol) and pyridine-3-sulfonyl chloride hydrochloride, prepared of Preparation 2, (0.070 g, 0.33 mmol) in 3 mL CH₂Cl₂ was stirred at room temperature overnight. The mixture was diluted with CH₂Cl₂ and the organic solution was washed with water and brine, dried over MgSO₄, filtered and concentrated in vacuo. The product was purified by flash chromatography on silica gel (10% EtOAc/hexanes to 30% EtOAc/hexanes) to afford the title compound of Step B. ¹H NMR (400 MHz, CDCl₃) δ 9.01 (s, 1H), 8.75 (d, 1H), 8.04 (d, 1H), 7.41 (dd, 1H), 7.23 (m, 4H), 4.30 (s, 2H), 3.62 (s, 3H), 3.08 (t, 2H), 2.55 (t, 2H), 2.19 (t, 2H), 1.10-1.58 (m, 12H), 0.87 (t, 3H); MS 447 (M+1).

### Step C: Ester Hydrolysis

7-((4-Butyl-benzyl)-(pyridine-3-sulfonyl)-amino)-heptanoic acid. A solution of 7-((4-butyl-benzyl)-(pyridine-3-sulfonyl)-amino)-heptanoic acid methyl ester prepared of Example 1, Step B (0.040 g, 0.158 mmol), in 2 mL MeOH and 0.5 mL 2N NaOH was stirred at room temperature overnight. The mixture was quenched with 2N HCl and was diluted with CH₂Cl₂. The organic layer was washed with 1 N HCl and water, dried over MgSO₄, filtered and concentrated in vacuo. The product was purified by flash chromatography on silica gel (2% MeOH/CH₂Cl₂ to 5% MeOH/CH₂Cl₂) to afford the title compound (42 mg). ¹H NMR (400 MHz, CDCl₃) δ 9.09 (s, 1H), 8.77 (d, 1H), 8.08 (d, 1H), 7.48 (dd, 1H), 7.09 (m, 4H), 4.32 (s, 2H), 3.12 (s, 2H), 2.55 (t, 2H), 2.25 (t, 2H), 1.12-1.58 (m, 12H), 0.88 (t, 3H); MS 431 (M−1).

### EXAMPLES 1a-1an

Examples 1a-1an were prepared from the appropriate starting materials in a manner analogous to the method of Example 1, with variations in reaction time, temperature, and reagents as noted.

### EXAMPLE 1a

### 7-(Benzenesulfonyl-(4-butyl-benzyl)-amino)-heptanoic acid

¹H NMR (400 MHz, CDCl₃) δ 7.83 (d, 2H), 7.51-7.59 (m, 3H), 7.11 (m, 4H), 4.28 (s, 2H), 3.07 (t, 2H), 2.57 (t, 2H), 2.24 (t, 2H), 1.51-1.59 (m, 2H), 1.44-1.49 (m, 2H), 1.27-1.35 (m, 4H), 1.08-1.15 (m, 4H), 0.91 (t, 3H); MS 430 (M−1).

### EXAMPLE 1b

### (3-(((1-Methyl-1H-indol-3-ylmethyl)-(pyridine-3-sulfonyl)-amino)-methyl)-phenyl)-acetic acid

¹H NMR (400 MHz, (CDCl₃) δ 8.93 (s, 1H), 8.66 (s, 1H), 7.96 (d, 1H), 7.39 (d, 1H), 7.01-7.37 (m, 9H), 6.77 (s, 1H), 4.56 (s, 2H), 4.41 (s, 2H), 3.66 (s, 3H), 3.52 (s, 2H); MS 448 (M−1).

### EXAMPLE 1c

### (3-(((5-Phenyl-furan-2-ylmethyl)-(pyridine-3-sulfonyl)-amino)-methyl)-phenyl)-acetic acid

¹H NMR (400 MHz, (CDCl₃) δ 8.02 (d, 1H), 7.22-7.34 (m, 12H), 6.42 (d, 1H), 6.17 (d, 1H), 4.45 (s, 2H), 4.40 (s, 2H), 3.60 (s, 2H); MS 461 (M−1).

### EXAMPLE 1d

### (3-(((5-Benzyl-pyridin-2-ylmethyl)-(pyridine-3-sulfonyl)-amino)-methyl)-phenyl)-acetic acid

Step A: Reaction time of 3.5 h at room temperature. ¹H NMR (400 MHz, CDCl₃) δ 8.97 (s, 1H), 8.71 (d, 1H), 8.15 (s, 1H), 7.98 (d, 1H), 7.44 (d, 1H), 7.04-7.34 (m, 10H), 4.54 (s, 2H), 4.43 (s, 2H), 3.87 (s, 2H), 3.50 (s, 2H); MS 486 (M−1).

### EXAMPLE 1e

### 3-(((4-Phenethylsulfanyl-benzyl)-(pyridine-3-sulfonyl)-amino)-methyl)-phenyl)-acetic acid

Step A: Reaction time of 4 h at room temperature. ¹H NMR (400 MHz, CDCl₃) δ 8.00 (d, 1H), 7.50 (bs, 1H), 6.90-7.38 (m, 15H), 4.31 (s, 4H), 3.49 (s, 2H), 3.11 (t, 2H), 2.87 (t, 2H); MS 531 (M−1).

### EXAMPLE 1f

### (3-(((3-Hydroxy-4-propoxy-benzyl)-(pyridine-3-sulfonyl)-amino)-methyl)-phenyl)-acetic acid

Step A: Reaction time of 3.5 h at room temperature. ¹H NMR (400 MHz, CDCl₃) δ 8.95 (s, 1H), 8.72 (s, 1H), 7.98 (d, 1H), 7.37 (m, 1H), 7.13-7.23 (m, 2H), 6.94-7.00 (m, 2H), 6.55-6.68 (m, 3H), 4.55 (s, 2H), 4.31 (s, 2H), 3.95 (t, 2H), 3.52 (s, 2H), 1.78 (m, 2H), 0.99 (t, 3H).

### EXAMPLE 1g

### (3-(((4-Pentyl-benzyl)-(pyridine-3-sulfonyl)-amino)-methyl)-phenyl)-acetic acid

Step A: Reaction time of 3.5 h at room temperature. ¹H NMR (400 MHz, CDCl₃) δ 8.98 (s, 1H), 8.74 (s, 1H), 8.00 (d, 1H), 7.39 (m, 1H), 7.14-7.26 (m, 2H), 6.95-7.05 (m, 6H), 4.35 (s, 4H), 3.54 (s, 2H), 2.54 (t, 2H), 1.56 (m, 2H), 1.29 (m, 4H), 0.88 (t, 3H); MS 465 (M−1).

### EXAMPLE 1h

### (3-(((4-Methylsulfamoyl-benzyl)-(pyridine-3-sulfonyl)-amino)-methyl)-phenyl)-acetic acid

Step A: Reaction time of 3.5 h at room temperature. ¹H NMR (400 MHz, CDCl₃) δ 9.06 (s, 1H), 8.85 (s, 1H), 8.16 (d, 1H), 7.53-7.64 (m, 3H), 6.91-7.26 (m, 6H), 4.39 (s, 2H), 4.35 (s, 2H), 3.50 (s, 2H), 2.63 (s, 3H); MS 488 (M−1).

### EXAMPLE 1i

### (3-(((4-Isopropoxy-benzyl)-(pyridine-3-sulfonyl)-amino)-methyl)-phenyl)-acetic acid

Step A: Reaction time of 3.5 h at room temperature. ¹H NMR (400 MHz, CDCl₃) δ 8.97 (s, 1H), 8.74 (s, 1H), 8.03 (m, 1H), 7.42 (m, 1H), 6.94-7.25 (m, 6H), 6.72 (m, 2H), 4.48 (m, 1H), 4.32 (m, 4H), 3.52 (s, 2H), 1.29 (t, 6H); MS 453 (M−1).

### EXAMPLE 1j

### (3-(((4-Chloro-thiophen-2-ylmethyl)-(pyridine-3-sulfonyl)-amino)-methyl)-phenyl-acetic acid

Step A: Reaction time of 3.5 h at room temperature. ¹H NMR (400 MHz, CDCl₃) δ 9.01 (s, 1H), 8.79 (s, 1H), 8.07 (d, 1H), 7.45 (m, 1H), 7.20-7.29 (m, 2H), 7.12 (d, 1H), 7.10 (s, 1H), 7.07 (s, 1H), 4.46 (s, 2H), 4.42 (s, 2H), 3.60 (s, 2H); MS 435 (M−1).

### EXAMPLE 1k

### (3-(((4-Butyl-benzyl)-(4-nitro-benzenesulfonyl)-amino)-methyl)-phenyl)-acetic acid

¹H NMR (400 MHz, CDCl₃) δ 8.23 (m, 2H), 7.85 (m, 2H), 7.15-7.25 (m, 2H), 695-7.02 (m, 6H), 4.32 (m, 4H), 3.53 (s, 2H), 2.52 (m, 2H), 1.51 (m, 2H), 1.30 (m, 2H), 0.89 (t, 3H); MS 495 (M−1).

### EXAMPLE 1l

### (3-(((4-Butyl-benzyl)-(4-cyano-benzenesulfonyl)-amino)-methyl)-phenyl)-acetic acid

¹H NMR (400 MHz, CDCl₃) δ 8.21 (d, 1H), 7.67-7.84 (m, 3H), 6.89-7.24 (m, 8H), 4.46 (s, 1H), 4.38 (s, 1H), 4.32 (m, 2H), 3.54 (s, 1H), 3.38 (s, 1H), 2.55 (m, 2H), 1.58 (m, 2H), 1.33 (m, 2H), 1.29 (s, 1H), 0.89 (t, 3H); MS 475 (M-1).

### EXAMPLE 1m

### (3-(((4-Butyl-benzyl)-(3-fluoro-benzenesulfonyl)-amino)-methyl)-phenyl)-acetic acid

¹H NMR (400 MHz, CDCl₃) δ 7.58 (m, 1H), 7.45 (m, 1H), 6.92-7.24 (m, 10H), 4.29 (m, 4H), 3.52 (d, 2H), 2.52 (d, 2H), 1.52 (m, 2H), 1.29 (m, 2H), 0.90 (m, 3H); MS 468 (M-1).

### EXAMPLE 1n

### (3-(((4-Butyl-benzyl)-(5-pyridin-2-yl-thiophene-3-sulfonyl)-amino)-methyl)-phenyl)-acetic acid

Step B: N,N-diisopropylethylamine was replaced by triethylamine. ¹H NMR (400 MHz, CDCl₃) δ 7.81 (m, 2H), 7.17-7.27 (m, 6H), 6.94-7.16 (m, 6H), 4.29 (d, 4H), 3.55 (s, 2H), 2.54 (m, 2H), 1.54 (m, 2H), 1.31 (m, 2H), 0.91 (t, 3H); MS 533 (M−1).

### EXAMPLE 1o

### (3-(((4-Butyl-benzyl)-(toluene-4-sulfonyl)-amino)-methyl)-phenyl)-acetic acid

Step B: N,N-diisopropylethylamine was replaced by triethylamine. ¹H NMR (400 MHz, CDCl₃) δ 7.71 (d, 2H), 7.24-7.29 (m, 2H), 7.11-7.19 (m, 2H), 6.87-7.01 (m, 2H), 4.26(d, 4H), 3.52 (s, 2H), 2.55 (m, 2H), 2.43 (s, 3H), 1.54 (m, 2H), 1.32 (m, 2H), 0.91 (t, 3H); MS 464 (M−1).

### EXAMPLE 1p

### (3-(((2,3-Dihydro-benzo[1.4]dioxin-6-ylmethyl)-(pyridine-3-sulfonyl)-amino)-methyl)-phenyl)-acetic acid

¹H NMR (400 MHz, CDCl₃) δ 8.98(s, 1H), 8.76 (s, 1H), 8.02 (d, 1H), 7.40 (m, 1H), 7.14-7.26 (m, 2H), 7.02 (d, 1H), 6.96 (s, 1H), 6.72 (d, 1H), 6.59 (m, 2H), 4.35 (s, 2H), 4.25 (s, 2H), 4.20 (s, 4H), 3.55 (s, 2H); MS 453 (M−1).

### EXAMPLE 1q

### (3-((Benzofuran-2-ylmethyl-(pyridine-3-sulfonyl)-amino)-methyl)-phenyl)-acetic acid

¹H NMR (400 MHz, CDCl₃) δ 9.05 (s, 1H), 8.66 (s, 1H), 8.04 (d, 1H), 7.11-7.42 (m, 9H), 6.44 (s, 1H), 4.45 (s, 1H), 4.39 (s, 1H), 3.59 (s, 1H); MS 435 (M−1).

### EXAMPLE 1r

### (3-(((4-Butyl-benzyl)-(1-methyl-1H-imidazole-4-sulfonyl)-amino)-methyl)-Phenyl)-acetic acid

¹H NMR (400 MHz, CDCl₃) δ 7.58 (s, 1H), 7.28 (s, 1H), 6.99-7.26 (m, 8H), 4.33 (d, 4H), 3.65 (s, 3H), 3.52 (s, 2H), 2.54 (t, 2H), 1.54 (m, 2H), 1.32. (m, 2H), 0.91 (t, 3H); MS 454 (M−1).

### EXAMPLE 1s

### (3-(((4-Imidazol-1-yl-benzyl)-(pyridine-3-sulfonyl)-amino)-methyl)-phenyl)-acetic acid

¹H NMR (400 MHz, CD₃OD) δ 9.45 (m, 1H), 9.44 (s, 1H), 9.03 (m, 1H), 8.91 (d, 1H), 8.19 (t, 1H), 8.04 (m, 1H), 7.77 (s, 1H), 7.61 (d, 2H), 7.53 (d, 2H), 7.11 (m, 4H), 4.70 (s, 2H), 4.51 (s, 2H), 3.33 (s, 2H); MS 461 (M−1).

### EXAMPLE 1t

### (3-(((Pyridine-3-sulfonyl)-(4-pyrimidin-2-yl-benzyl)-amino)-methyl)-phenyl)-acetic acid

¹H NMR (400 MHz, CDCl₃) δ 9.10 (s, 1H), 8.80 (m, 3H), 8.14 (d, 1H), 8.02 (d, 2H), 7.47 (m, 1H), 7.06-7.25 (m, 6H), 6.83 (s, 1H), 4.40 (s, 2H), 4.33 (s, 2H), 3.41 (s, 2H); MS 473 (M−1).

### EXAMPLE 1u

### (3-(((Pyridine-3-sulfonyl)-(4-thiazol-2-yl-benzyl)-amino)-methyl)-phenyl)-acetic acid

¹H NMR (400 MHz, CDCl₃) δ 9.11 (s, 1H), 8.85 (s, 1H), 8.15 (d, 1H), 7.87 (s, 2H), 7.63 (d, 2H), 7.51 (m, 1H), 7.37 (s, 1H), 7.07-7.27 (m, 6H), 6.83 (s, 1H), 4.37 (s, 2H), 4.33 (s, 2H), 3.41 (s, 2H).

### EXAMPLE 1v

### (3-(((1-Methyl-1H-imidazole-4-sulfonyl)-(4-thiazol-2-yl-benzyl)-amino)-methyl)-phenyl)-acetic acid

Step B: N,N-diisopropylethylamine was replaced by triethylamine. ¹H NMR (400 MHz, CDCl₃) δ 7.85 (s, 1H),7.59 (m, 2H), 7.47 (s, 1H), 7.34 (s, 1H), 7.07-7.25 (m, 6H), 6.88 (s, 1H), 4.46 (s, 2H), 4.38 (s, 2H), 3.77 (s, 3H), 3.40 (s, 2H); MS 483 (M−1).

### EXAMPLE 1w

### (3-(((4-Dimethylamino-benzyl)-(pyridine-3-sulfonyl)-amino]-methyl)-phenyl)-acetic acid

Step A: Reaction time of 4 h at room temperature. Step B: N,N-diisopropylethylamine was replaced by triethylamine. ¹H NMR (400 MHz, CD₃OD) δ 8.09 (d, 1H), 7.09-7.16 (m, 2H), 6.93-6.99 (m, 7H), 6.65 (d, 2H), 5.36 (s, 2H), 4.32 (s, 2H), 4.27 (s, 2H), 2.89 (s, 6H); MS 438 (M−1).

### EXAMPLE 1x

### (3-(((4-Cyclohexyl-benzyl)-(pyridine-3-sulfonyl)-amino)-methyl)-phenoxy)-acetic acid

Step B: N,N-diisopropylethylamine was replaced by triethylamine. ¹H NMR (400 MHz, CDCl₃) δ 8.95 (s, 1H), 8.73 (d, 1H), 8.00 (d, 1H), 7.39 (m, 1H), 7.17 (t, 1H), 7.13 (d, 0.2H), 7.08 (d, 2H), 6.81 (d, 1H), 6.73 (d, 1H), 6.61 (s, 1H), 4.54 (s, 2H), 4.34 (s, 4H), 2.43 (m, 1H), 1.81 (d, 4H), 1.37 (t, 4H), 1.23 (m, 1H); MS 495 (M+1), 493 (M−1).

### EXAMPLE 1y

### (3-(((2-(3.5-Dichloro-phenoxy)-ethyl)-(pyridine-3-sulfonyl)-amino)-methyl)-phenoxy)-acetic acid

Step B: N,N-diisopropylethylamine was replaced by triethylamine. ¹H NMR (400 MHz, CDCl₃) δ 9.07 (s, 1H), 8.78 (d, 1H), 8.12 (d, 1H), 7.47 (m, 1H), 7.25 (m, 1H), 6.82-6.91 (m, 4H), 6.53 (s, 2H), 4.61 (s, 2H), 4.47 (s, 2H), 3.91 (t, 2H), 3.54 (t, 2H); MS 511 (M+1), 509 (M−1).

### EXAMPLE 1z

### (3-(((4-Dimethylamino-benzyl)-(pyridine-3-sulfonyl)-amino)-methyl)-phenoxy)-acetic acid

Step B: N,N-diisopropylethylamine was replaced by triethylamine. ¹H NMR (400 MHz, CDCl₃) δ 8.91 (s, 1H), 8.79 (m, 1H), 8.04 (d, 1H), 7.43 (m, 1H), 7.16 (t, 1H), 6.94 (d, 2H), 6.81 (d, 2H), 6.64 (d, 2H), 6.49 (s, 1H), 4.51 (s, 2H), 4.28 (s, 4H), 2.91 (s, 6H);
MS 456 (M+1), 454 (M−1).

### EXAMPLE 1aa

### (3-(((4-tert-Butyl-benzyl)-(pyridine-3-sulfonyl)-amino)-methyl)-phenoxy)-acetic acid

Step B: N,N-diisopropylethylamine was replaced by triethylamine. ¹H NMR (400 MHz, CDCl₃) δ 8.95 (s, 1H), 8.74 (s, 1H), 7.99 (d, 1H), 7.39 (m, 1H), 7.25 (m, 2H), 7.15 (t, 1H), 7.04 (d, 2H), 6.81 (d, 1H), 6.72 (d, 1H), 6.62 (s, 1H), 4.55 (s, 2H), 4.35 (s, 4H), 1.27 (s, 9H); MS 469 (M+1), 467 (M−1).

### EXAMPLE 1ab

### (3-(((3-(3-Chloro-phenyl)-propyl)-(pyridine-3-sulfonyl)-amino)-methyl)-phenoxy) acetic acid

Step B: N,N-diisopropylethylamine was replaced by triethylamine. ¹H NMR (400 MHz, CDCl₃) δ 8.98 (s, 1H), 8.77 (d, 1H), 8.07 (d, 1H), 7.48 (m, 1H), 7.21 (m., 2H), 6.9,1 (s, 1H), 6.86 (m, 3H), 6.78 (s, 1H), 4.61 (s, 2H), 4.31 (s, 2H), 3.15 (t, 2H), 2.43 (t, 2H), 1.68 (m, 2H); MS 475 (M+1), 473 (M−1).

### EXAMPLE 1ac

### (3-(((4-tert-Butyl-benzyl)-(1-methyl-1H-imidazole-4-sulfonyl)-amino)-methyl)-phenoxy)-acetic acid

Step B: N,N-diisopropylethylamine was replaced by triethylamine. ¹H NMR (400 MHz, CDCl₃) δ 7.66 (s, 1H), 7.08-7.31 (m, 6H), 6.70-6.78 (m, 3H), 4.54 (s, 2H), 4.35 (s, 4H), 3.68 (s, 3H), 1.27 (s, 9H); MS 469.9 (M−1).

### EXAMPLE 1ad

### (3-(((4-Cyclohexyl-benzyl)-(pyridine-3-sulfonyl)-amino)-methyl)-phenyl)-acetic acid

Step B: N,N-diisopropylethylamine was replaced by triethylamine. ¹H NMR (400 MHz, CDCl₃) δ 8.98 (bs, 1H), 8.75 (bs, 1H), 7.98 (d, 1H), 7.39 (bs, 1H), 6.97-7.25 (m, 8H), 4.36 (d, 4H), 3.54 (s, 2H), 2.44 (s, 1H), 1.72-1.82 (m, 4H), 1.24-1.36 (m, 5H); MS 476.9 (M−1).

### EXAMPLE 1ae

### (3-(((11-Methyl-1H-imidazole-4-sulfonyl)-(4-phenoxy-benzyl)-amino)-methyl)-phenyl)-acetic acid

Step B: N,N-diisopropylethylamine was replaced by triethylamine. ¹H NMR (400 MHz, CDCl₃) δ 7.52 (s, 1H), 7.06-7.37 (m, 10H), 6.94 (d, 2H), 6.83 (d, 2H), 4.38 (s, 4H), 3.71 (s, 3H), 1.72-1.82 (m, 4H), 3.56 (s, 2H); MS 490 (M−1).

### EXAMPLE 1af

### (3-(((4-Phenoxy-benzyl)-(pyridine-3-sulfonyl)-amino)-methyl)-phenyl)-acetic acid

¹H NMR (400 MHz, CDCl₃) δ 9.00 (bs, 1H), 8.76 (bs, 1H), 8.04 (d, 1H), 7.41 (t, 1H), 7.35 (m, 1H), 6.86-7.32 (m, 10H), 6.84 (d, 2H), 4.37 (d, 4H), 3.54 (s, 2H); MS 487 (M-1).

### EXAMPLE 1aq

### (3-(((4-(2-Oxo-Pyrrolidin-1-yl)-benzyl)-(pyridine-3-sulfonyl)-amino)-methyl)-phenyl)-acetic acid

Step B: N,N-diisopropylethylamine was replaced by triethylamine. ¹H NMR (400 MHz, CDCl₃) δ 9.06 (bs, 1H), 8.80 (bs, 1H), 8.14 (m, 1H), 7.47 (m, 1H), 6.96-7.26 (m, 7H), 4.28 (m, 4H), 3.78 (m, 2H), 3.35 (m, 2H), 2.59 (m, 2H), 2.11 (m, 2H); MS 478 (M−11).

### EXAMPLE 1ah

### (3-((Benzo[1,3]dioxol-5-ylmethyl-(pyridine-3-sulfonyl)-amino)-methyl)-phenyl)-acetic acid

¹H NMR (400 MHz, CDCl₃) δ 8.98 (s, 1H), 8.76 (s, 1H), 8.04 (d, 1H), 7.41(m, 1H), 7.14-7.20 (m, 2H), 7.00 (d, 1H), 6.94 (s, 1H), 6.64 (t, 2H), 6.55 (d, 1H), 4.34 (s, 2H), 4.26 (s, 2H), 3.54 (s, 2H); MS 439 (M−1).

### EXAMPLE 1ai

### (3-(((1-Methyl-1H-imidazole-4-sulfonyl)-(4-pyrimidin-5-yl-benzyl)-amino)-methyl)-phenyl)-acetic acid

Step B: N,N-diisopropylethylamine was replaced by triethylamine. ¹H NMR (400 MHz, CDCl₃) δ 9.18 (s, 1H), 8.91 (s, 2H), 7.05-7.54 (m, 11H), 4.49 (s, 2H), 4.40 (s, 2H), 3.75 (s, 3H), 3.55 (s, 2H); MS 476 (M−1).

### EXAMPLE 1aj

### (3-(((Pyridine-3-sulfonyl)-(4-pyrimidin-5-yl-benzyl)-amino)-methyl)-phenyl)-acetic acid

¹H NMR (400 MHz, CD₃OD) δ 9.17 (s, 1H), 9.01 (s, 1H), 8.77 (s, 1H), 7.57 (m, 4H), 7.45 (d, 2H), 7.05-7.16 (m, 5H), 4.48 (s, 2H), 4.43 (s, 2H), 3.45 (s, 2H).

### EXAMPLE 1ak

### (3-(((4-Pyrazin-2-yl-benzyl)-(pyridine-3-sulfonyl)-amino)-methyl)-phenyl)-acetic acid

¹H NMR (400 MHz, DMSO-d₆) δ 9.18 (s, 1H), 9.02 (s, 1H), 8.83 (d, 1H), 8.68 (s, 1H), 8.57 (s, 1H), 8.25 (d, 1H), 7.96 (d, 2H), 7.60 (m, 1H), 7.26 (d, 2H), 7.15 (m, 2H), 7.05 (m, 2H), 4.42 (s, 2H), 4.41 (s, 2H).

### EXAMPLE 1al

### (3-(((1-Methyl-1H-imidazole-4-sulfonyl)-(4-pyrimidin-2-yl-benzyl)-amino)-methyl)-phenyl)-acetic acid

¹H NMR (400 MHz, CDCl₃) δ 8.78 (d, 2H), 7.94 (d, 2H), 7.54 (s, 1H), 7.44 (s, 1H), 7.22-7.03 (m, 6H), 6.87 (s, 1H), 4.45 (s, 2H), 4.39 (s, 2H), 3.73 (s, 3H), 3.38 (s, 2H); MS 476 (M−1).

### EXAMPLE 1am

### (3-(((4-Butyl-benzyl)-phenylmethanesulfonyl-amino)-methyl)-phenyl)-acetic acid

¹H NMR (400 MHz, CDCl₃) δ 7.31-6.96 (m, 13H), 4.13 (s, 2H), 4.05 (s, 2H), 4.03 (s, 2H), 3.62 (s, 2H), 2.60 (t, 2H), 1.58 (m, 2H), 1.33 (m, 2H), 0.91 (t, 3H); MS 464 (M−1).

### EXAMPLE 1an

### 5-(3-((Pyridine-3-sulfonyl)-(4-thiazol-2-yl-benzyl)-amino)-propyl)-thiophene-2-carboxylic acid

Step A: Triethylamine was replaced with N,N-diisopropylethylamine. ¹H NMR (400 MHz, CDCl₃) δ 9.18 (d, 1H), 8.82 (d, 1H), 8.05 (d, 1H), 7.73-7.20 (m, 8H), 6.60 (d, 1H), 4.35 (s, 2H), 3.22 (t, 2H), 2.70 (t, 2H), 1.85-1.70 (m, 2H).

### EXAMPLE 2

### (3-(((2-(3-Chloro-phenoxy)-ethyl)-(Pyridine-3-sulfonyl)-amino]-methyl)-phenyl)-acetic acid

### Step A: Alkylation

(3-(((2-(3-Chloro-phenoxy)-ethyl)-(pyridine-3-sulfonyl)-amino)-methyl)-phenyl)-acetic acid methyl ester. To a solution of sodium hydride (60% in mineral oil, 0.016 g, 0.3996 mmol) in 2 mL DMF was added (3-((pyridine-3-sulfonylamino)-methyl)-phenyl)-acetic acid methyl ester (from Preparation 14, 0.096 g, 0.333 mmol) at 0° C. and the reaction was stirred at room temperature for 30 minutes. After cooling to 0° C., 1-(2-bromo-ethoxy)-3-chloro-benzene (from Preparation 29, 0.094 g, 0.399 mmol) was added and the reaction was stirred at room temperature overnight. The DMF was removed in vacuo. The residue was diluted with EtOAc and the organic solution was washed with water and brine, dried over MgSO₄, filtered and concentrated in vacuo. The product was purified by flash chromatography on silica gel (0.5% MeOH/CH₂Cl₂ to 2% MeOH/CH₂Cl₂) to afford the title compound of Step A (0.025 g). MS 475 (M+1).

### Step B: Ester Hydrolysis

(3-(((2-(3-Chloro-Phenoxy)-ethyl)-(pyridine-3-sulfonyl)-amino)-methyl)-phenyl)-acetic acid. A solution of the compound of Example 2, Step A (0.025 g, 0.053 mmol), in 2 mL MeOH and 0.5 mL 2N NaOH was stirred at room temperature overnight. The mixture was quenched with 2N HCl and was diluted with CH₂Cl₂. The organic layer was washed with 1 N HCl and water, dried over MgSO₄, filtered, and concentrated in vacuo. The product was purified by flash chromatography on silica gel (2% MeOH/CH₂Cl₂ to 5% MeOH/CH₂Cl₂) to afford the title compound (20 mg). ¹H NMR (400 MHz, CDCl₃) δ 9.05 (s, 1H), 8.77 (d, 1H), 8.11 (d, 1H), 7.43 (m, 1H), 7.08-7.27 (m, 5H), 6.89 (d, 1H), 6.62 (s, 1H), 6.55 (d, 1H), 4.51 (s, 2H), 3.95 (t, 2H), 3.59 (s, 4H); MS 495 (M−2).

### EXAMPLES 2a-2c

Examples 2a-2c were prepared from the appropriate starting materials in a manner analogous to the method of Example 2.

### EXAMPLE 2a

### Trans-(3-(((3-(3,5-Dichloro-phenyl)-allyl)-(pyridine-3-sulfonyl)-amino)-methyl)-phenyl)-acetic acid

¹H NMR (400 MHz, CDCl₃) δ 9.08 (bs, 1H), 8.81 (bs, 1H), 8.11 (d, 1H), 7.48 (bs, 1H), 7.12-7.28 (m, 4H), 6.98 (s, 2H), 6.19 (d, 1H), 5.86 (m, 1H), 4.38 (s, 2H), 3.93 (d, 2H), 3.58 (s, 2H).

### EXAMPLE 2b

### (3-(((2-(3.5-Dichloro-phenoxy)-ethyl)-(pyridine-3-sulfonyl)-amino)-methyl)-phenyl)-acetic acid

¹H NMR (400 MHz, CDCl₃) δ 8.96 (bs, 1H), 8.70 (bs, 1H), 8.04 (d, 1H), 7.41 (m, 1H), 7.24-7.09 (m, 4H), 6.86 (s, 1H), 6.47 (s, 2H), 4.44 (s, 2H), 3.86 (m, 2H), 3.49 (s, 2H), 3.31 (m, 2H).

### EXAMPLE 2c

### (3-(((4-(1-Hydroxy-hexyl)-benzyl)-(pyridine-3-sulfonyl)-amino)-methyl)-phenyl)-acetic acid

¹H NMR (400 MHz, CDCl₃) δ 8.91 (bs, 1H), 8.72 (bs, 1H), 8.03 (d, 1H), 7.40 (bs, 1H), 7.16-6.99 (m, 7H), 6.81 (s, 1H), 4.57 (t, 1H), 4.29 (s, 4H), 3.43 (m, 2H), 1.70 (m, 1H), 1.61 (m, 1H), 1.32-1.16 (m, 8H), 0.82 (t, 3H).

### EXAMPLE 3

### 5-(3-((2-Benzylsulfanyl-ethyl)-(pyridine-3-sulfonyl)-amino)-propyl)-thiophene-2 carboxylic acid

### Step A: Reductive Amination

5-(3-(2-Benzylsulfanyl-ethylamino)-propyl)-thiophene-2-carboxylic acid tert-butyl ester. Step A was performed in a manner analogous to the method of Step A of Example 1.

### Step B: Amide Formation

5-(3-((2-Benzylsulfanyl-ethyl)-(pyridine-3-sulfonyl)-amino)-propyl)-thiophene-2-carboxylic acid tert-butyl ester. Step B was performed in a manner analogous to the method of Step B of Example 1, except triethylamine was used in place of N,N-diisopropylethylamine.

### Step C: Ester Hydrolysis

5-(3-((2-Benzylsulfanyl-ethyl)-(pyridine-3-sulfonyl)-amino)-propyl)-thiophene-2-carboxylic acid.TFA. A solution of 5-(3-((2-benzylsulfanyl-ethyl)-(pyridine-3-sulfonyl)-amino-propyl)-thiophene-2-carboxylic acid tert-butyl ester prepared of Example 3, Step B (0.038 g) in 1 mL CH₂Cl₂ was cooled to 0° C. and 1 mL TFA was added. The mixture was warmed to room temperature and was stirred for 1 h. The CH₂Cl₂ and TFA were removed by evaporation, azeotroping with added CH₂Cl₂ to yield the title compound (46.3 mg). MS 475 (M−1).

Examples 3a-3i were prepared from the appropriate starting materials in a manner analagous to the method of Example 3 with variations thereto noted.

### EXAMPLE 3a

### 5-(3-((2-(3-Chloro-phenylsulfanyl)-ethyl)-(pyridine-3-sulfonyl)-amino)-propyl) thiophene-2-carboxylic acid

¹H NMR (400 MHz, CD₃OD) δ 8.93 (s, 1H), 8.78 (d, 1H), 8.21 (d, 1H), 7.64 (m, 1H), 7.57 (s, 1H), 7.35 (s, 1H), 7.19-7.28 (m, 3H), 6.87 (s, 1H), 3.16-3.35 (m, 6H), 2.87 (t, 2H), 1.89 (t, 2H); MS 497,499 (M+).

### EXAMPLE 3b

### (3-(((Pyridine-3-sulfonyl)-(4-thiazol-2-yl-benzyl)-amino)-methyl)-phenoxy)-acetic acid.2TFA

¹H NMR (400 MHz, CDCl₃) δ 9.40 (bs, 1H), 8.98 (s, 1H), 8.84 (s, 1H), 8.28 (m, 1H), 8.10 (s, 1H), 7.78 (m, 2H), 7.68 (m, 1H), 7.51 (s, 1H), 7.24 (m, 3H), 7.12 (t, 1H), 6.77 (m, 1H), 6.48 (s, 1H), 4.53 (s, 2H), 4.45 (s, 2H), 4.34 (s, 2H); MS 494 (M−1).

### EXAMPLE 3c

### (3-(((Pyridine-3-sulfonyl)-(4-pyrimidin-2-yl-benzyl)amino)-methyl)-phenoxy)-acetic acid.2HCl

The TFA salt was converted to the HCl salt by stirring in 2 equivalents 1 N HCl followed by removal of water and drying in vacuo. ¹H NMR (400 MHz, CD₃OD) 8.9.00 (d, 2H), 8.78 (d, 1H), 8.25 (d, 2H), 8.08 (t, 1H), 7.60 (t, 1H), 7.42 (m, 3H), 7.11 (m, 1H), 6.81 (d, 1H), 6.72 (m, 3H), 4.65 (s, 2H), 4.60 (s, 2H), 4.49 (s, 2H).

### EXAMPLE 3d

### (3-(((1-Methyl-1H-imidazole-4-sulfonyl)-(4-thiazol-2-yl-benzyl)-amino)-methyl)-Phenoxy)-acetic acid.2TFA

¹H NMR (400 MHz, CD₃OD) δ 7.93 (s, 1H), 7.85 (d, 1H), 7.76 (d, 2H), 7.70 (s, 1H), 7.60 (d, 1H), 7.26 (d, 2H), 7.09 (t, 1H), 6.75 (d, 2H), 6.68 (s, 1H), 4.51 (s, 2H), 4.41 (s, 2H), 4.35 (s, 2H), 3.76 (s, 3H); MS 498 (M+).

### EXAMPLE 3e

### (3-(((Pyridine-3-sulfonyl)-(4-pyridin-2-yl-benzyl)-amino)-methyl)-phenoxy)-acetic acid. HCl

No triethylamine was used in Step A. The TFA salt was converted to the HCl salt by stirring in 2 equivalents 1 N HCl followed by removal of water and drying in vacuo. MS 490 (M+1), 488 (M−1).

### EXAMPLE 3f

### (3-(((1-Methyl-1H-imidazole-4-sulfonyl)-(4-pyridin-2-yl-benzyl)-amino)-methyl)-phenoxy)-acetic acid.HCl

No triethylamine was used in Step A. The TFA salt was converted to the HCl salt by stirring in 2 equivalents 1 N HCl followed by removal of water and drying in vacuo. MS 493(M+1), 491 (M−1).

### EXAMPLE 3g

### (3-(((Pyridine-3-sulfonyl)-(4-pyridin-3-yl-benzyl)-amino)-methyl)-phenoxy)-acetic acid.HCl

No triethylamine was used in Step A. The TFA salt was converted to the HCl salt by stirring in 2 equivalents 1 N HCl followed by removal of water and drying in vacuo. MS 490 (M+1), 488 (M−1).

### EXAMPLE 3h

### (3-(((1-Methyl-1H-imidazole-4-sulfonyl)-(4-pyridin-3-yl-benzyl)-amino)-methyl)-Phenoxy)-acetic acid.HCl

No triethylamine was used in Step A. The TFA salt was converted to the HCl salt by stirring in 2 equivalents 1 N HCl followed by removal of water and drying in vacuo. MS 493 (M+1), 491 (M−1).

### EXAMPLE 3i

### (3-(((Pyridine-3-sulfonyl)-(4-pyridin-4-yl-benzyl)-amino)-methyl)-phenoxy)-acetic acid.HCl

No triethylamine was used in Step A. The TFA salt was converted to the HCl salt by stirring in 2 equivalents 1 N HCl followed by removal of water and drying in vacuo MS 490 (M+1), 488 (M−1).

### EXAMPLE 4

### 5-(3-((3-(3-Chloro-phenyl)-propyl)-(pyridine-3-sulfonyl)-amino)-propyl)-thiophene-2-carboxylic acid

### Step A: Sulfonamide Formation

5-(3-((3-(3-Chloro-phenyl)-propyl)-(pyridine-3-sulfonyl)-amino)-propyl)-thiophene-2-carboxylic acid methyl ester. A solution of 5-(3-(3-(3-chloro-phenyl)-propylamino)-propyl)-thiophene-2-carboxylic acid methyl ester (from Preparation 8, 0.0855 g, 0.243 mmol), triethylamine (0.0541 g 0.534 mmol), and pyridine-3-sulfonyl chloride hydrochloride (from Preparation 2, 0.0572 g, 0.267 mmol) in 10 mL CH₂Cl₂ combined at 0° C. was stirred at room temperature overnight. The organic solution was washed with water, saturated NaHCO₃ and brine, dried over MgSO₄, filtered and concentrated in vacuo to afford the title compound of Step A as an oil. MS 494 (M+1).

### Step B: Ester Hydrolysis

5-(3-((3-(3-Chloro-phenyl)-propyl)-(pyridine-3-sulfonyl)-amino)-propyl)-thiophene-2-carboxylic acid. A solution of 5-(3-((3-(3-chloro-phenyl)-propyl)-(pyridine-3-sulfonyl)-amino)-propyl)-thiophene-2-carboxylic acid methyl ester prepared of Example 4, Step B (0.119 g, 0.241 mmol), in 5 mL EtOH and 0.72 mL 1N NaOH was stirred at room temperature overnight. The reaction mixture was adjusted to pH 6.2 and the layers were separated. The organic solution was washed with water, dried over MgSO₄, filtered and concentrated in vacuo to afford the title compound (16 mg). ¹H NMR (400 MHz, CDCl₃) δ 8.00 (d, 1H, J=8), 7.70 (d, 1H, J=4), 7.30-7.60 (m, 6H), 6.75 (d, 1H, J=4), 3.20 (m, 4H), 2.95 (t, 2H, J=7), 2.60 (t, 2H, J=7), 1.70-2.00 (m, 4H); MS 478 (M+1), 476 (M−1).

### EXAMPLES 4a-4h

Examples 4a-4h were prepared from the appropriate starting in a manner analogous to the method of Example 4.

### EXAMPLE 4a

### 5-(3-((3-(3-Chloro-phenyl)-propyl)-(4-methoxy-benzenesulfonyl)-amino)-propyl)-thiophene-2-carboxylic acid

¹H NMR (400 MHz, CDCl₃) δ 7.70 (d, 1H, J=7), 7.00-7.40 (m, 8H), 6.80 (d, 1H, J=4), 3.89 (s, 3H), 3.10 (m, 4H), 2.95 (t, 2H, J=7), 2.50 (t, 2H, J=7), 1.70-2.00 (m, 2H); MS 508 (M+1), 506 (M−1).

### EXAMPLE 4b

### 5-(3-((Benzo[1,2.5]thiadiazole-4-sulfonyl)-(3-(3-chloro-phenyl)-propyl)-amino)-propyl)-thiophene-2-carboxylic acid

¹H NMR (400 MHz, CDCl₃) δ 7.00-7.70 (m, 8H), 6.70 (d, 1H, J=4), 3.05 (m, 4H), 2.90 (t, 2H, J=7), 2.54 (t, 2H, J=7), 1.72-1.92 (m, 2H); MS 536 (M+), 535 (M−1).

### EXAMPLE 4c

### 5-(3-(Benzenesulfonyl-(3-(3-chloro-phenyl)-propyl)-amino)-propyl)-thiophene-2-carboxylic acid

¹H NMR (400 MHz, CDCl₃) δ 6.70-7.92 (m, 11H), 3.26 (m, 4H), 3.05 (m, 4H), 2.73 (m, 2H), 2.50 (m, 2H), 1.70 (m, 2H); MS 578(M+1), 576 (M−1).

### EXAMPLE 4d

### 5-(3-((3-(3-Chloro-phenyl)-propyl)-phenylmethanesulfonyl-amino)-propyl)-thiophene-2-carboxylic acid

¹H NMR (400 MHz, CDCl₃) δ 7.50 (d, 1H, J=4), 7.00-7.40 (m, 9H), 6.85 (d, 1H, J=4), 3.00 (m, 4H), 2.60 (m, 2H), 2.40 (m, 2H), 1.60-1.80 (m, 2H); MS 490 (M−1).

### EXAMPLE 4e

### 5-(3-((3-(3-Chloro-phenyl)-propyl)-(pyridine-3-sulfonyl)-amino)-propyl)-furan-2-carboxylic acid

¹H NMR (400 MHz, CDCl₃) δ 9.00 (m, 1H), 8.70 (m, 1H), 8.00 (d, 1H, J=6), 7.50 (m, 1H), 6.80-7.04 (m, 6H), 3.20 (m, 4H), 2.78 (m, 2H), 2.50 (m, 2H), 1.62-2.00 (m, 4H);
MS 463 (M+1), 461. (M−1).

### EXAMPLE 4f

### 5-(3-((3-(3-Chloro-phenyl)-propyl)-(naphthalene-2-sulfonyl)-amino)-propyl)-thiophene-2-carboxylic acid

¹H NMR (400 MHz, CDCl₃) δ 8.40 (d, 1H, J=2), 7.00-8.00 (m, 11H), 6.80 (d, 1H, J=4), 3.20 (m, 4H), 2.82 (t, 2H, J=7), 2.60 (t, 2H, J=7), 1.80-2.00 (m, 2H); MS 528.9 (M+1).

### EXAMPLE 4a

### 5-(3-((3-(3-Chloro-phenyl)-propyl)-(naphthalene-1-sulfonyl)-amino)-propyl)-thiophene-2-carboxylic acid

¹H NMR (400 MHz, CDCl₃) δ 8.60 (d, 1H. J=5), 6.95-8.22 (m, 11H), 6.70 (d, 1H, J=₄), 3.20 (m, 4H), 2.40 (t, 2H, J=7), 1.72-1.95 (m, 4H); MS 528.9 (M+1).

### EXAMPLE 4h

### 5-(3-((2-Acetylamino-4-methyl-thiazole-5-sulfonyl)-(3-(3-chloro-phenyl)-propyl)-amino)-propyl)-thiophene-2-carboxylic acid

¹H NMR (400 MHz, CDCl₃) δ 7.61 (d, 1H, J=4), 7.00-7.30 (m, 4H), 3.60 (d, 1H, J=3.8), 2.80 (t, 2H, J=7.0), 2.60 (t, 2H, J=6.8), 2.40 (s, 3H), 2.30 (s, 3H), 1.70-2.00 (m, 4H); MS 556 (M+1), 554 (M−1).

### EXAMPLE 5

### 5-(3-((3-(3-Chloro-phenyl)-propyl)-(pyridine-3-carbonyl)-amino)-propyl)-thiophene-2-carboxylic acid

### Step A: Amide Formation

5-(3-((3-(3-Chloro-phenyl)-propyl)-(pyridine-3-carbonyl)-amino)-Propyl)-thiophene-2-carboxylic acid methyl ester. A solution of 5-(3-(3-(3-chloro-phenyl)-propylamino)-propyl)-thiophene-2-carboxylic acid methyl ester (from Preparation 8, 0.075 g, 0.213 mmol), DCC (0.0483 g 0.234 mmol) and nicotinic acid (0.0289 g, 0.234 mmol) in 10 mL CH₂Cl₂ was stirred at room temperature overnight. The mixture was filtered and the filtrate was concentrated in vacuo. The residue was dissolved in 15 mL EtOAc and the insolubles were removed via filtration. The organic solution was washed with water followed by brine, dried over MgSO₄, filtered, and concentrated in vacuo to afford the title compound of Step A as an oil (113 mg). MS 457 (M+).

### Step B: Ester Hydrolysis

Step B was performed in a manner analogous to the method of Step B of Example 4.

¹H NMR (400 MHz, CDCl₃) δ 8.60 (d, 1H, J=8), 6.80-7.70 (m, 8H), 6.60 (d, 1H, J=4), 3.25 (m, 4H), 2.80 (m, 2H), 2.45 (m, 2H), 1.60-2.05 (m, 4H); MS 443 (M+1), 441 (M-1).

### EXAMPLES 5a-5b

Examples 5a-5b were prepared from the appropriate starting in a manner analogous to the method of Example 5.

### EXAMPLE 5a

### 5-(3-((3-(3-Chloro-phenyl)-propyl)-(pyridin-2-yl-acetyl)-amino)-propyl)-thiophene-2-carboxylic acid

¹H NMR (400 MHz, CDCl₃) δ 8.60 (m, 1H), 7.00-7.80 (m, 8H), 6.60 (m, 1H), 4.00 (s, 2H), 3.32 (m, 4H), 2.72 (m, 2H), 2.50 (m, 2H), 1.70-2.00 (m, 4H); MS 457 (M+1), 455 (M−1).

### EXAMPLE 5b

### 5-(3-((3-(3-Chloro-phenyl)-propyl)-(Pyridin-3-yl-acetyl)-amino)-propyl)-thiophene-2-carboxylic acid

¹H NMR (400 MHz, CDCl₃) δ 7.60-7.80 (m, 2H), 7.00-7.50 (m, 7H), 6.70 (d, 1H, J=4), 3.60 (s, 2H), 3.10-3.40 (m, 4H), 2.80 (m, 2H), 2.60 (m, 2H), 1.70-2.00 (m, 4H); MS 457 (M+1), 455 (M−1).

### EXAMPLE 6

### 5-(3-((2-Chloro-benzenesulfonyl)-(3-(3-chloro-phenyl)-propyl)-amino)-propyl)-thiophene-2-carboxylic acid

### Step A: Amide Formation

5-(3-((2-Chloro-benzenesulfonyl)-(3-(3-chloro-phenyl)-propyl)-amino)-propyl)-thiophene-2-carboxylic acid tert-butyl ester. A stock solution of 5-(3-(3-(3-chloro-phenyl)-propylamino)-propyl)-thiophene-2-carboxylic acid tert-butyl ester (from Preparation 9, 0.10 g, 0.254 mmol) in 10 mL CH₂Cl₂ was prepared and 1 mL of the solution (0.010 g, 0.0254 mmol) was added to a 1 dram vial. To this was added triethylamine (0.78 mL, 0.056 mmol) and 2-chloro-benzenesulfonyl chloride (0.0059 g, 0.028 mmol). The reaction was stirred overnight at room temperature and was diluted with 2 mL CH₂Cl₂. The organic solution was washed with 3 mL of 5.5% aqueous HCl solution (2×) and 3 mL saturated bicarbonate solution (2×). The organic layer was dried with MgSO₄ and was concentrated to yield the title compound of Step A (10 mg).

### Step B: Ester Hydrolysis

5-(3-((2-Chloro-benzenesulfonyl)-(3-(3-chloro-phenyl)-propyl)-amino)-propyl)-thiophene-2-carboxylic acid. A, solution of 5-(3-((2-chloro-benzenesulfonyl)-(3-(3-chloro-phenyl)-propyl)-amino)-propyl)-thiophene-2-carboxylic acid tert-butyl ester prepared of Example 6, Step A (0.010 g, 0.010 mmol) in 4N HCl in 1,4 dioxane (3 mL) and the reaction was stirred overnight at room temperature. HCl (g) was bubbled in until reaction was determined to be complete by thin layer chromatography. The reaction mixture was concentrated in vacuo. The resulting organic residue was azeotroped with CCl₄ to produce a powder (5 mg). ¹H NMR (400 MHz, CDCl₃) δ 8.00 (d, 1H. J=4), 7.00-7.72 (m, 8H,), 6.75 (d, 1H, J=4), 3.20-3.40 (m, 4H), 2.81 (m, 2H), 2.52 (m, 2H), 1.90 (m, 2H), 1.80 (m, 2H), 1.20 (m, 2H); MS 509.9 (M−1).

### EXAMPLES 6a-6j

Examples 6a-6j were prepared from the appropriate starting material in a manner analogous to the method of Example 6.

### EXAMPLE 6a

### 5-(3-((3-(3-Chloro-phenyl)-propyl)-(2,5-dimethyl-benzenesulfonyl)-amino)-propyl)-thiophene-2 carboxylic acid

¹H NMR (400 MHz, CDCl₃) δ 7.70 (d, 1H, J=7), 7.00-7.40 (m, 7H), 6.80 (d, 1H, J=4), 3.32 (m, 4H), 2.50 (s, 3H), 2.36 (s, 3H), 1.84 (m, 2H), 1.75 (m, 2H), 1.22 (m, 2H); MS 506.1 (M+1), 504.1 (M−1).

### EXAMPLE 6b

### 5-(3-((3-(3-Chloro-phenyl)-propyl)-(2.4-dioxo-1,2,3,4-tetrahydro-quinazoline-6-sulfonyl)-amino)-propyl)-thiophene-2-carboxylic acid

¹H NMR (400 MHz, CDCl₃) δ 6.80-7.92 (m, 9H), 3.20 (m, 4H), 2.80 (m, 2H), 1.75-2.00 (m, 4H), 1.20 (m, 2H); MS 594.0 (M−1+Cl).

### EXAMPLE 6c

### 5-(3-((4-(2-Carboxy-benzoylamino)-butane-1-sulfonyl)-(3-(3-chloro-phenyl)-propyl)-amino)-propyl)-thiophene-2-carboxylic acid

¹H NMR (400 MHz, CDCl₃) δ 7.70 (d, 1H, J=6), 7.62 (d, 1H, J=4), 7.55 (d, 1H, J=8), 7.45-7.20 (m, 6H), 6.80-6.90 (m, 10H), 3.22 (m, 4H), 2.70 (m, 2H), 2.60 (m, 2H), 1.80-2.00 (m, 4H), 1.22 (m, 2H); MS 620.1 (M−1).

### EXAMPLE 6d

### 5-(3-((3-(3-Chloro-phenyl)-propyl)-(4-(3,5-dioxo-4,5-dihydro-3H-[1,2,4]triazin-2-yl)-benzenesulfonyl)-amino)-propyl)-thiophene-2-carboxylic acid

¹H NMR (400 MHz, CDCl₃) δ 7.60-7.92 (m, 4H), 6.80 (m, 7H), 3.22 (m, 4H), 2.80 (m, 2H), 2.60 (m, 2H), 1.82 (m, 2H), 1.22 (m, 2H); MS 587.1 (M−1).

### EXAMPLE 6e

### 5-(3-((3-(3-Chloro-Phenyl)-propyl)-(2-methoxycarbonyl-benzenesulfonyl)-amino)-propyl)-thiophene-2-carboxylic acid

¹H NMR (400 MHz, CDCl₃) δ 7.75 (d, 1H, J=4), 7.00-7.70 (m, 8H), 6.85 (d, 1H, J=4), 3.90 (s, 3H), 3.31 (m, 4H), 2.70 (m, 2H), 2.50 (m, 2H), 1.82-2.00 (m, 4H), 1.20 (m, 2H); MS 534.1 (M−1).

### EXAMPLE of

### 5-(3-((4-Bromo-benzenesulfonyl)-(3-(3-chloro-phenyl)-propyl)-amino)-propyl)-thiophene-2-carboxylic acid

¹H NMR (400 MHz, CDCl₃) δ 7.75 (d, 1H, J=4), 7.00-7.70 (m, 8H), 6.80 (d, 1H, J=4), 3.10 (m, 4H), 2.86 (m, 2H), 2.55 (m, 2H), 1.90 (m, 2H), 1.80 (m, 2H); MS 557.9 (M+1), 555.9 (M−1).

### EXAMPLE 6g

### 5-(3-((3-(3-Chloro-phenyl)-propyl)-(4-(1,1-dimethyl-propyl)-benzenesulfonyl)-amino)-Propyl)-thiophene-2-carboxylic acid

¹H NMR (400 MHz, CDCl₃) δ 7.95 (d, 1H, J=4), 7.00-7.80 (m, 8H), 6.80 (d, 1H, J=4), 3.20 (m, 4H), 2.80 (m, 2H), 2.50 (m, 2H), 1.30 (s, 3H), 1.70-1.90 (m, 4H), 1.55 (m, 2H), 0.60 (t, 3H, J=7); MS 548 (M+1).

### EXAMPLE 6h

### 5-(3-((3-(3-Chloro-phenyl)-propyl)-(3.5-dimethyl-isoxazole-4-sulfonyl)-amino)-propyl)-thiophene-2-carboxylic acid

¹H NMR (400 MHz, CDCl₃) δ 6.95-7.40 (m, 4H), 6.80 (d, 1H, J=8), 6.75 (d, 1H, J=8), 2.91 (m, 2H), 2.60 (s, 3H), 2.40 (m, 2H), 2.20 (s, 3H), 1.72-1.92 (m, 4H), 1.20 (m, 2H); MS 495 (M−1).

### EXAMPLE 6i

### 5-(3-((3-(3-Chloro-phenyl)-propyl)-(2,5-dimethoxy-benzenesulfonyl)-amino)-propyl)-thiophene-2-carboxylic acid

¹H NMR (400 MHz, CDCl₃) δ 7.70 (d, 1H, J=4), 7.00-7.50 (m, 7H), 6.80 (d, 1H, J=4), 4.00 (s, 3H), 3.80 (s, 3H), 3.25 (m, 4H), 2.85 (m, 2H), 2.52 (m, 2H), 1.70-2.00 (m, 2H); MS 538.1 (M+1), 536.1 (M−1).

### EXAMPLE 6j

### 5-(3-((3-(3-Chloro-phenyl)-propyl)-(2-fluoro-benzenesulfonyl)-amino)-propyl)-thiophene-2-carboxylic acid

¹H NMR (400 MHz, CDCl₃) δ 7.00-8.00 (m, 9H), 6.80 (d, 1H, J=7.2), 3.30 (m, 4H), 2.85 (m, 2H), 2.55 (m, 2H), 1.70-2.00 (m, 4H), 1.20 (m, 2H); MS 494.1 (M−1).

### EXAMPLE 7

### 5-(3-(1-(3-(3-Chloro-phenyl)-propyl)-3-ethyl-ureido)-propyl)-thiophene-2-carboxylic acid

### Step A: Isocyanate Addition

5-(3-(1-(3-(3-Chloro-phenyl)-propyl)-3-ethyl-ureido)-propyl)-thiophene-2-carboxylic acid tert-butyl ester. A stock solution of 5-(3-(3-(3-chloro-phenyl)-propylamino)-propyl)-thiophene-2-carboxylic acid tert-butyl ester (from Preparation 9, 0.10 g, 0.254 mmol) in 10 mL CH₂Cl₂ was prepared and 1 mL (0.010 g, 0.0254 mmol) was added to a 1 dram vial. Triethylamine (0.7 mL, 0.051 mmol) and ethyl isocyanate (0.004 g, 0.051 mmol) were added and the mixture was stirred overnight at room temperature. The solution was diluted with 2 mL CH₂Cl₂. The organic solution was washed with 3 mL of 5.5% aqueous HCl solution (2×) followed by 3 mL saturated bicarbonate solution (2×). The organic layer was dried with MgSO₄ and was concentrated to yield the title compound of Step A (10 mg).

### Step B: Ester Hydrolysis

Step B was performed in a manner analogous to the method of Step B of Example 6.

¹H NMR (400 MHz, CDCl₃) δ 7.70 (d, 1H, J=4), 7.00-7.40 (m, 4H), 6.80 (d, 1H, J=4), 3.20 (m, 6H), 2.80 (m, 2H), 2.60 (m, 2H), 1.80-2.00 (m, 4H), 1.05 (t, 3H, J=7); MS 409.1 (M+1), 407.1 (M−1).

### EXAMPLES 7a-7j

Examples 7a-7j were prepared from the appropriate starting materials in a manner analogous to the method of Example 7.

### EXAMPLE 7a

### 5-(3-(1-(3-(3-Chloro-phenyl)-propyl)-3-isopropyl-ureido)-propyl)-thiophene-2-carboxylic acid

¹H NMR (400 MHz, CDCl₃) δ 7.70 (d, 1H, J=4), 7.00-7.40 (m, 4H), 6.80 (d, 1H, J=4), 3.20 (m, 4H), 2.85 (m, 2H), 2.60 (m, 2H), 1.75-2.00 (m, 4H), 1.05 (d, 6H, J=7), MS 423.1 (M+1), 421.1 (M−1).

### EXAMPLE 7b

### 5-(3-(1-(3-(3-Chloro-phenyl)-propyl)-3-phenyl-ureido)-propyl)-thiophene-2-carboxylic acid

¹H NMR (400 MHz, CDCl₃) δ 7.75 (d, 1H, J=7), 7.00-7.50 (m, 9H), 6.80 (d, 1H, J=4), 3.20 (m, 4H), 2.90 (m, 2H), 2.60 (m, 2H), 1.80-2.00 (m, 4H); MS 457.1 (M+1), 455.2 (M−1).

### EXAMPLE 7c

### 5-(3-(1-(3-(3-Chloro-phenyl)-propyl)-3-(3,4-dichloro-phenyl)-ureido)-propyl)-thiophene-2-carboxylic acid

¹H NMR (400 MHz, CDCl₃) δ 6.80-7.60 (m, 9H), 3.20 (m, 4H), 2.90 (m, 2H), 2.60 (m, 2H), 1.86-2.00 (m, 4H); MS 527.0 (M+1), 525.0 (M−1).

### EXAMPLE 7d

### 5-(3-(1-(3-(3-Chloro-Phenyl)-propyl)-3-propyl-ureido)-propyl)-thiophene-2-carboxylic acid

¹H NMR (400 MHz, CDCl₃) δ 7.70 (d, 1H, J=4), 7.00-7.30 (m, 4H), 6.80 (d, 1H, J=4), 3.20-3.30 (m, 5H), 2.95 (t, 2H, J=7), 2.60 (t, 2H, J=7), 1.70-2.00 (m, 4H), 0.95 (t, 3H, J=7); MS 423 (M+1), 421 (M−1).

### EXAMPLE 7e

### 5-(3-(3-(4-Chloro-phenyl)-1-(3-(3-chloro-phenyl)-propyl)-ureido)-propyl)-thiophene-2-carboxylic acid

¹H NMR (400 MHz, CDCl₃) δ 7.70 (d, 1H, J=4), 7.00-7.30 (m, 8H), 6.80 (d, 1H, J=4), 3.22 (m, 4H), 2.90 (m, 2H), 2.65 (m, 2H), 1.69-2.02 (m, 4H); MS 491(M+1), 489 (M-1).

### EXAMPLE 7f

### 5-(3-(1-(3-(3-Chloro-phenyl)-propyl)-3-(2,3-dichloro-phenyl)-ureido)-propyl) thiophene-2-carboxylic acid

¹H NMR (400 MHz, CDCl₃) δ 7.70 (bs, 1H), 7.00-7.30 (m, 7H), 6.80 (bs, 1H), 3.20 (m, 4H), 2.80 (m, 2H), 2.60 (m, 2H), 1.75-2.00 (m, 4H); MS 527 (M+1), 525.1 (M−1).

### EXAMPLE 7g

### 5-(3-(1-(3-(3-Chloro-phenyl)-propyl)-3-(3,5-dichloro-phenyl)-ureido)-propyl)-thiophene-2-carboxylic acid

¹H NMR (400 MHz, CDCl₃) δ 7.70 (d, 1H, J=₄), 7.00-7.30 (m, 7H), 6.80 (d, 1H, J=4), 3.20 (m, 4H), 2.80 (m, 2H), 2.60 (m, 2H), 1.70-2.00 (m, 4H); MS 527.1 (M+1), 525.1 (M−1).

### EXAMPLE 7h

### 5-(3-(1-(3-(3-Chloro-phenyl)-propyl)-3-(2,6-difluoro-phenyl)-ureido)-propyl)-thiophene-2-carboxylic acid

¹H NMR (400 MHz, CDCl₃) δ 7.70 (d, 1H, J=4), 7.00-7.30 (m, 7H), 6.80 (d, 1H, J=4), 3.20 (m, 4H), 2.86 (m, 2H), 2.65 (m, 2H), 1.73-1.95 (m 4H); MS 493.1 (M+1), 491.1 (M−1).,

### EXAMPLE 7i

### 5-(3-(1-(3-(3-Chloro-phenyl)-propyl)-3-(4-fluoro-phenyl)-ureido)-propyl)-thiophene-2 carboxylic acid

¹H NMR (400. MHz, CDCl₃) δ 7.70 (bs, 1H), 7.00-7.60 (m, 8H), 6.80 (bs, 1H), 3.30 (m, 4H), 2.90 (m, 2H), 2.60 (m, 2H), 1.80-2.00 (m, 4H); MS 475.1 (M+1), 473.1 (M−1).

### EXAMPLE 7j

### 5-(3-(3-Butyl-1-(3-(3-chloro-phenyl)-propyl)-ureido)-propyl)-thiophene-2-carboxylic acid

¹H NMR (400 MHz, CDCl₃) δ 7.70 (bs, 1H), 7.00-7.20 (m, 4H), 6.80 (bs, 1H), 3.20 (m, 6H), 2.90 (m, 2H), 2.60 (m, 2H), 1.70-2.00 (m, 4H), 0.95 (t, 3H, J=6.8); MS 437.2 (M+1), 435.2 (M−1).

### EXAMPLE 8

### 5-(3-((3-(3-Chloro-phenyl)-propyl)-(Pyrrolidine-1-carbonyl)-amino)-propyl)-thiophene-2-carboxylic acid

### Step A: Amide Formation

5-(3-(1-(3-(3-Chloro-phenyl)-propyl)-3-ethyl-ureido)-propyl)-thiophene-2-carboxylic acid tert-butyl ester. A stock solution of 5-(3-(3-(3-chloro-phenyl)-propylamino)-propyl)-thiophene-2-carboxylic acid tert-butyl ester (from Preparation 9, 0.10 g, 0.254 mmol) in 10 mL CH₂Cl₂ was prepared and 1 mL (0.010 g, 0.0254 mmol) was added to a 1 dram vial. Triethylamine (0.7 mL, 0.051 mmol) and ethyl isocyanate (0.004 g, 0.051 mmol) were added and the reaction was stirred overnight at room temperature. The reaction was diluted with 2 mL CH₂Cl₂ and the organic solution was washed with 3 mL of 5.5% aqueous HCl solution (2×) followed by 3 mL saturated bicarbonate solution (2×). The organic layer was dried with MgSO₄ and concentrated to yield the title compound of Step A (10 mg).

### Step B: Ester Hydrolysis

Step B was performed in a manner analogous to the method of Step B of Example 6.

¹H NMR (400 MHz, CDCl₃) δ 7.70 (d, 1H, J=4), 7.00-7A40 (m, 4H), 6.80 (d, 1H, J=4), 3.20 (m, 8H), 2.80 (m, 2H), 2.60 (m, 2H), 1.70-2.00 (m, 8H), 1.20 (m, 4H); MS 435.1 (M+1), 433.2 (M−1).

### EXAMPLES 8a-8c

Examples 8a-8c were prepared from the appropriate starting material in a manner analogous to the method of Example 8.

### EXAMPLE 8a

### 5-(3-((3-(3-Chloro-phenyl)-Propyl)-(morpholine-4-carbonyl)-amino)-propyl)-thiophene-2-carboxylic acid

¹H NMR (400 MHz, CDCl₃) δ 7.65 (d, 1H, J=4), 7.00-7.40 (m, 4H), 6.80 (d, 1H, J=4), 3.60 (m, 4H), 3.00-3.20 (m, 8H), 2.80 (m, 2H), 2.60 (m, 2H), 1.70-2.00 (m, 4H); MS 451.1 (M+1), 449.2 (M−1).

### EXAMPLE 8b

### 5-(3-((3-(3-Chloro-phenyl)-propyl)-isopropoxycarbonyl-amino)-propyl)-thiophene-2-carboxylic acid

¹H NMR (400 MHz, CDCl₃) δ 6.70 (d, 1H, J=4), 7.00-7.30 (m, 4H), 6.80 (d, 1H, J=4), 3.20 (m, 4H), 2.80 (t, 2H, J=6.7), 2.60 (t, 2H, J=6.7), 1.80-2.00 (m, 4H), 1.01 (d, 6H); MS 424 (M+1), 422 (M−1).

### EXAMPLE 8c

### 5-(3-((3-(3-Chloro-phenyl)-propyl)-Propoxycarbonyl-amino)-propyl)-thiophene-2-carboxylic acid

¹H NMR (400 MHz, CDCl₃) δ 7.70 (bs, 1H), 7.00-7.30 (m, 4H), 6.80 (bs, 1H), 4.00 (t, 2H, J=6.8), 3.30 (m, 4H), 2.80 (m, 2H), 2.60 (m, 2H), 1.40-2.00 (m, 6H), 0.90 (t, 3H, J=7); MS 424 (M+1), 422.2 (M−1).

### EXAMPLE 9

### (3-(((4-Butyl-benzyl)-(pyridine-3-sulfonyl)-amino)-methyl)-phenyl)-acetic acid

### Step A: Reductive Amination

(3-((4-Butyl-benzylamino)-methyl)-phenyl)-acetic acid methyl ester. A solution of 4-butyl-benzylamine (from Preparation 15, 0.918 g, 6 mmol) in MeOH was added to 4N HCl in dioxane (0.75 mL, 3 mmol) followed by addition of (3-formyl-phenyl)-acetic acid methyl ester (from Preparation 13, 0.534 g, 3.0 mmol). NaCNBH₃ (0.194 mL, 3 mmol) was added and the reaction was stirred at room temperature overnight. The mixture was diluted with EtOAc and 2N NaOH was added. The organic solution was dried over MgSO₄, filtered, and concentrated in vacuo. The product was purified via flash chromatography (50% hexanes, 50% EtOAc, 0.1% Et₃N) to afford the title compound of Step A. ¹H NMR (400 MHz, CDCl₃) δ 7.08-7.38 (m, 8H), 3.75 (s, 2H), 3.73 (s, 2H), 3.70 (s, 3H), 3.62 (s, 2H), 2.61 (t, 2H), 1.58 (m, 2H), 1.37 (m, 2H), 0.92 (t, 3H); MS 326 (M+1).

### Step B: Amide Formation

(3-(((4-Butyl-benzyl)-(pyridine-3-sulfonyl)-amino)-methyl)-phenyl)-acetic acid methyl ester. Step B was performed in a manner analogous to the method of Step B of Example 1 to provide the title compound.

### Step C: Ester Hydrolysis

(3-(((4-Butyl-benzyl)-(pyridine-3-sulfonyl)-amino)-methyl)-phenyl)-acetic acid. Step C was performed in a manner analogous to the method of Step C of Example 1 to provide the title compound. ¹H NMR (400 MHz, CDCl₃) δ 8.99 (bs, 1H), 8.74 (bs, 1H), 7.99 (d, 1H), 7.36 (bs, 1H), 7.20-7.25 (m, 2H), 6.95-7.19 (m, 6H), 4.33 (s, 0.4H), 3.3.54 (s, 2H), 2.54 (m, 2H), 1.54 (m, 2H), 1.32 (m, 2H), 0.91 (t, 3H).

### EXAMPLES 9a-9d

Examples 9a-9d were prepared from the appropriate starting materials in a manner analogous to the method of Example 9.

### EXAMPLE 9a

### (3-((Benzenesulfonyl-(4-butyl-benzyl)-amino)-methyl)-phenyl)-acetic acid

¹H NMR (400 MHz, CDCl₃) δ 7.83 (d, 2H), 7.46-7.58 (m, 3H), 7.24 (s, 1H), 7.14 (m, 2H), 6.86-6.98 (m, 5H), 4.29 (d, 4H), 3.51 (s, 2H), 2.52 (t, 2H), 1.53 (m, 2H), 1.30 (m, 2H), 0.90 (t, 2H); MS 450 (M−1).

### EXAMPLE 9b

### (3-(((4-Butyl-benzyl)-(thiophene-2-sulfonyl)-amino)-methyl)-phenyl)-acetic acid

¹H NMR (400 MHz, CDCl₃) δ 7.53 (m, 2H), 7.16 (m, 2H), 6.89-7.14 (m, 7H), 4.27 (d, 0.4H), 3.52 (s, 2H), 2.49 (t, 2H), 1.51 (m, 2H), 1.29 (m, 2H), 0.88 (t, 2H); MS 456 (M-1).

### EXAMPLE 9c

### (3-(((4-Acetylamino-benzenesulfonyl)-(4-butyl-benzyl)-amino)-methyl)-phenyl)-acetic acid

¹H NMR (400 MHz, CDCl₃) δ 7.69 (m, 2H), 7.49 (d, 2H), 7.06-7.23 (m, 6H), 6.91 (d, 1H), 6.68 (s, 1H), 4.30 (d, 4H), 3.44 (s, 2H), 2.54 (t, 2H), 2.17 (s, 3H), 1.54 (m, 2H), 1.29 (m, 2H), 0.89 (t, 2H); MS 507(M−1).

### EXAMPLE 9d

### (3-(((Benzo[1,2,5]oxadiazole-4-sulfonyl)-(4-butyl-benzyl)-amino)-methyl)-phenyl)-acetic acid

¹H NMR (400 MHz, CDCl₃) δ 7.94 (d, 1H), 7.88 (d, 2H), 7.36 (t, 1H), 7.07 (s, 2H), 6.90-6.96 (m, 6H), 53 (d, 4H), 3.46 (s, 2H), 2.46 (t, 2H), 1.47 (m, 2H), 1.26 (m, 2H), 0.88 (t, 2H); MS 4.92 (M−1).

### EXAMPLE 10

### (3-(((1-Methyl-1H-imidazole-4-sulfonyl)-(4-pyrimidin-2-yl-benzyl)-amino)-methyl)-phenoxy)-acetic acid.HCl

### Step A: Reductive Amination

(3-((4-Pyrimidin-2-yl-benzylamino)-methyl)-phenoxy)-acetic acid t-butyl ester. Step A was performed in a manner analogous to the method of Step A of Example 1.

### Step B: Amide Formation

(3-(((1-Methyl-1H-imidazole-4-sulfonyl)-(4-pyrimidin-2-yl-benzyl)-amino)-methyl)-phenoxy)-acetic acid tert-butyl ester. Step B was performed in a manner analogous to the method of Step B of Example 1 using triethylamine in place of N,N-diisopropylethylamine as base.

### Step C: Ester Hydrolysis

(3-(((1-Methyl-1H-imidazole-4-sulfonyl)-(4-pyrimidin-2-yl-benzyl)-amino)-methyl)-phenoxy)-acetic acid.HCl. A solution of (3-(((1-methyl-1H-imidazole-4-sulfonyl)-(4-pyrimidin-2-yl-benzyl)-amino)-methyl)-phenoxy)-acetic acid tert-butyl ester prepared of Example 10, Step B (0.094 g, 0.17 mmol) in 1N HCl in diethyl ether was stirred for 20 minutes as a precipitate formed. To the mixture was added 1 mL water and 1 mL dioxane and the reaction was stirred for 3 hours. The solvent was removed in vacuo, azeotroping with ethanol to yield the title compound as a solid (54 mg). ¹H NMR (400 MHz, CD₃OD) δ 9.09 (m, 2H), 8.95 (bs, 1H), 8.24 (d, 2H), 8.04 (s, 1H), 7.71 (s, 1H), 7.44 (d, 2H), 7.13 (m, 1H), 6.82 (d, 1H), 6.76 (d, 1H), 6.69 (s, 1H), 4.61 (s, 2H), 4.54 (s, 2H), 4.46 (s, 2H), 3.92 (s, 3H); MS 494 (M+1).

### Preparation 1

7-Amino-heptanoic acid methyl ester hydrochloride. A solution of 7-amino-heptanoic acid (3.0 g, 21.0 mmol), in 25 mL MeOH and 2.4 mL concentrated HCl was heated at reflux for 4 hours and was stirred at room temperature for 60 h. The mixture was concentrated in vacuo to afford the title compound (3.3 g). ¹H NMR (400 MHz, CD₃OD) δ 3.62 (s, 3H), 2.89 (m, 2H), 2.31 (t, 2H), 1.62 (m, 4H), 1.37 (m, 4H).

### Preparation 2

Pyridine-3-sulfonyl chloride hydrochloride. The title compound was prepared using the method described by Karaman, R. and coworkers J. Am. Chem. Soc. 114, 12, 1992, 48894898.

### Preparation 3

3-(3-Chloro-phenyl)-propionaldehyde. A solution of. 1-chloro-3-iodobenzene (9.63 g, 40.38 mmol), allyl alcohol (5.86 g, 100.96 mmol), sodium bicarbonate (8.48 g, 100.96 mmol), tetrabutylammonium chloride (11.22 g, 40.38 mmol), and Pd(OAc)₂ (317 mg, 1.413 mmol) in 25 mL DMF was stirred at 50° C. for 16 h. The mixture was cooled to room temperature, diluted with water, and the aqueous solution was washed with EtOAc. The organic solution was washed with water followed by brine, dried over MgSO₄, filtered and concentrated in vacuo. The product was purified via flash chromatography on silica gel (9:1 hexanes:EtOAc) to afford the title compound as an oil (5.04 g).

### Preparation 4

### 5-(3-Oxo-propyl)-thiophene-2-carboxylic acid tert-butyl ester

### Step A: Ester Formation

5-Bromo-thiophene-2-carboxylic acid tert-butyl ester. To a solution of anhydrous MgSO₄ (11.60 g, 96.4 mmol) in 100 mL CH₂Cl₂ was added concentrated H₂SO₄ (1.45 mL, 24.1 mmol) and the mixture was stirred for 15 minutes followed by addition of 5-bromo-thiophene-2-carboxylic acid (5.0 g, 24.1 mmol). After stirring for 1 minute, tert-butanol (11.6 g, 20 mmol) was added and the reaction was stirred at room temperature for 16 h. The reaction was quenched with saturated NaHCO₃. The layers were separated, the aqueous layer was extracted with CH₂Cl₂, and the combined organic layers were dried over MgSO₄. The organic solution was concentrated to give a clear oil which was purified via medium pressure chromatography (3% EtOAc in hexanes) to afford the title compound of Step A(4.97 g). ¹H NMR (400 MHz, CDCl₃) δ 7.45 (d, 1H), 7.02 (d, 1H), 1.54 (s, 9H).

### Step B: Aldehyde Formation

5-(3-Oxo-propyl)-thiophene-2-carboxylic acid tert-butyl ester. To a solution of 5-bromo-thiophene-2-carboxylic acid tert-butyl ester prepared of the method of Preparation 4, Step A (0.50 g, 1.89 mmol) in 5 mL DMF was added allyl alcohol (0.51 mL, 7.57 mmol) followed by NaHCO₃ (0.397 g, 4.72 mmol), tetrabutylammonium chloride (0.525 g, 1.89 mmol), and palladium acetate (0.021 g, 0.094 mmol). The reaction was placed in an oil bath heated to 65° C. and was heated to 90° C. for 2 h. The mixture was diluted with EtOAc and 25 mL water and the solids were removed by filtration through Celite®. The layers were separated, and the organic solution was washed with water (4×), dried over MgSO₄ and concentrated to a dark yellow oil which was purified via medium pressure chromatography (7:1 hexanes:EtOAc) to afford the title compound (0.190 g). ¹H NMR (400, MHz, CDCl₃) δ 9.80 (s, 1H), 7.51 (d, 1H), 6.78 (d, 1H), 3.14 (t, 2H), 2.86 (t, 2H), 1.54, (s, 9H).

### Preparation 5

### 5-(3-Amino-propyl)-thiophene-2-carboxylic acid methyl ester

### Step A

5-(3-tert-Butoxycarbonylamino-prop-1-vnyl)-thiophene-2-carboxylic acid methyl ester. A mixture of prop-2-ynyl-carbamic acid tert-butyl ester (from Preparation 41, 1.67 g, 0.011 mmol), 5-bromo-thiophene-2-carboxylic acid methyl ester (2.50 g, 0.011 mmol), tetrakistriphenylphosphine(0) palladium (0.622 g, 0.0538 mmol), CuI (0.102 g, 0.538 mmol) and triethylamine (1.57 mL, 0.011 mmol) in 50 mL acetonitrile was heated at reflux for 16 h. The reaction was cooled to room temperature, diluted with 75 mL EtOAc, washed with 5.5% HCl, water and brine, dried over MgSO₄, filtered and concentrated in vacuo to an oil. The product was purified via flash chromatography (9:1 to 4:1 hexanes:EtOAc) to afford the title compound of Step A as an oil (2.06 g). MS 313 (M+18).

### Step B

5-(3-tert-Butoxycarbonylamino-propyl)-thiophene-2-carboxylic acid methyl ester. A solution of 5-(3-tert-butoxycarbonylamino-prop-1-ynyl)-thiophene-2-carboxylic acid methyl ester prepared of Preparation 5, Step A (2.06 g) and 10% Pd/C (1.03 g) in 50 mL MeOH was hydrogenated on a Parr shaker at 50 psi H₂ for 16 h. The reaction was filtered through Celite® with the aid of MeOH and the filtrate was concentrated in vacuo to afford the title compound of Step B as a solid (1.93 g). MS 317 (M+18).

### Step C

5-(3-Amino-propyl)-thiophene-2-carboxylic acid methyl ester. A solution of 5-(3-tert-butoxycarbonylamino-propyl)-thiophene-2-carboxylic acid methyl ester prepared of Preparation 5, Step B (0.118 g, 0.5 mmol) in 50 mL MeOH was cooled to 0° C. and was saturated with HCl (g). The reaction was stirred at room temperature for 90 minutes. The solution was concentrated to a solid which was partitioned between EtOAc and saturated NaHCO₃. The layers were separated, and the combined organic layers were washed with brine, dried over MgSO₄, filtered and concentrated in vacuo to afford the title compound as an oil (399 mg). MS 200 (M+1).

### Preparation 6

5-(3-Amino-propyl)-furan-2-carboxylic acid methyl ester hydrochloride salt. The compound of Preparation 6 was prepared from the appropriate starting materials in a manner analogous to the method of Preparation 5 with the following exceptions: (1) the hydrogenation performed in Step B was carried out for 5.5 h; and (2) in Step C, the reaction was stirred for 16 h at room temperature and was concentrated in vacuo to provide the title compound as the hydrochloride salt.

### Preparation 7

### 5-(3-Amino-propyl)-thiophene-2-carboxylic acid tert-butyl ester

### Step A

Prop-2-ynyl-carbamic acid benzyl ester. To a solution of propargylamine (6.4 g, 71.2 mmol) in pyridine (100 mL) was added benzylchloroformate (13.37 g, 78.2 mmol) in 100 mL CH₂Cl₂ over 0.5 h. The reaction was stirred for 16 h and the volatiles were removed in vacuo. The residue was dissolved in EtOAc and the organic solution was washed with water (2×). The organic solution was washed with dilute aqueous HCl followed by saturated NaHCO₃. The organic solution was dried over MgSO₄, filtered, and concentrated in vacuo to provide the title compound of Step A (4.43 g).

### Step B

5-(3-Benzyloxycarbonylamino-prop-1-ynyl)-thiophene-2-carboxylic acid tert-butyl ester. The title compound of Step B was prepared from the appropriate starting material in a manner analagous to the method used in Step A of Preparation 5.

### Step C

5-(3-Amino-propyl)-thiophene-2-carboxylic acid tert-butyl ester. To a solution of 5-(3-benzyloxycarbonylamino-prop-1-ynyl)-thiophene-2-carboxylic acid tert-butyl ester prepared of Preparation 7, Step B (1.0 g, 2.69 mmol) in 15 mL MeOH and 2.69 mL 1 N HCl (aq) was added Pd(OH)₂. The mixture was hydrogenated on a Parr shaker at 45 psi H₂ for 16 h. The mixture was filtered through Celite®, the catalyst was replaced, and the reaction was shaken for another 6 h. The mixture was filtered through Celite® and concentrated in vacuo. The residue was chased with CCl₄ and was triturated with Et₂O. The product was isolated as a solid (360 mg).

### Preparation 8

5-(3-(3-(3-Chloro-phenyl)-propylamino)-propyl)-thiophene-2-carboxylic acid methyl ester. A solution of 5-(3-amino-propyl)-thiophene-2-carboxylic acid methyl ester (from Preparation 5, Step C, 0.118 g, 0.5 mmol) and N,N-diisopropylethylamine (0.071 g, 0.55 mmol) in 10 mL MeOH was stirred at room temperature for 30 minutes and 3-(3-chloro-phenyl)-propionaldehyde (from Preparation 3, 0.093 g, 0.55 mmol) was added. The mixture was stirred for 90 minutes. The reaction was cooled to 0° C., NaBH₄ (30.3 mg, 0.801 mmol) was added and the mixture was stirred for 30 minutes. The reaction was quenched with 1:1 NaHCO₃:H₂O and was washed with CH₂Cl₂. The CH₂Cl₂ extracts were washed with brine, dried over MgSO₄, filtered, and concentrated in vacuo to afford the title compound as an oil (171 mg). MS 352 (M+1).

### Preparations 9-10

The compounds of Preparations 9 and 10 were prepared from the appropriate starting materials in a manner analogous to the method of Preparation 8.

### Preparation 9

### 5-(3-(3-(3-Chloro-phenyl)-propylamino)-propyl)-thiophene-2-carboxylic acid tert-butyl ester

### Preparation 10

5-(3-(3-(3-Chloro-phenyl)-propylamino)-propyl)-furan-2-carboxylic acid methyl ester MS 336 (M+1).

### Preparation 11

(3-Formyl-phenoxy)-acetic acid methyl ester. A mixture of (3-formyl-phenoxy)-acetic acid (3.6 g, 20.0 mmol), potassium carbonate (3.30 g, 23.9 mmol) and methyl iodide (1.86 g, 30.0 mmol) in 25 mL DMF was heated to 110° C. for 2 hours and was stirred at room temperature for 16 h. The mixture was diluted with water and the aqueous solution was extracted with EtOAc. The organic solution was washed with water, dried over MgSO₄, filtered and concentrated in vacuo. The product was purified via silica gel chromatography (4:1 hexanes: EtOAc) to afford the title compound as a pale yellow oil (3.4 g). ¹H NMR (400 MHz, CDCl₃) δ 9.94 (s, 1H), 7.48 (m, 2H), 7.33 (s, 1H), 7.23 (m, 1H), 4.68 (s, 2H), 3.79 (s, 3H).

### Preparation 12

### 3-(3-Chloro-phenyl)-propylamine

### Step A

3-(3-Chloro-phenyl)-acrylamide. A solution of 3-(3-chloro-phenyl)-acrylic acid (Aldrich, 15.0 g, 82.15 mmol) in 50 mL thionyl chloride was heated at reflux for 30 minutes. The excess thionyl chloride was removed via distillation at atmospheric pressure. The residue was azeotroped with benzene in vacuo to give 17.288 g of an orange oil. The oil was dissolved in 25 mL CH₂Cl₂ and the solution was added slowly to liquid NH₃ (20 mL, 80.07 mmol) in CHCl₃ (50 mL) at −78° C. The resulting suspension was warmed to room temperature and was concentrated in vacuo to afford the title compound of Step A as a gray solid (19.38 g). ¹H NMR (400 MHz, CD₃OD) δ 7.57 (s, 1H), 7.45 (m, 2H), 7.36 (m, 1H), 6.64 (d, 1H); MS 182 (M+1), 180 (M−1).

### Step B

3-(3-Chloro-phenyl)-propylamine. A 1.0 M solution of LiAlH₄ in THF (6.0 mL) was added dropwise to a suspension of 3-(3-chloro-phenyl)-acrylamide prepared of Preparation 12, Step A (1.0 g, 5.51 mmol) in 30 mL THF at 0° C. The reaction was warmed to room temperature and was stirred for 5 h. An additional 4 mL of 1 M LiAlH₄ was added and the reaction was stirred for 18 h. An additional 2 mL of 1, M LiAlH₄ was added and the reaction was stirred for 24 h. The reaction mixture was quenched by dropwise, addition of water. The mixture was concentrated in vacuo to remove THF and was diluted with water. The aqueous solution was extracted with EtOAc. The organic solution was washed with water, dried over MgSO₄, filtered and concentrated in vacuo. The residue was dissolved in CHCl₃ and the organic solution was washed with 1 M HCl. The aqueous solution was basified to pH 11 with 1 M NaOH and the product was extracted into CHCl₃. The organic solution was dried over MgSO₄, filtered and concentrated in vacuo to afford the title compound as a yellow oil (0.134 g). ¹H NMR (400 MHz, CDCl₃) δ 7.20-7.22 (m, 3H),7.16 (m, 1H), 2.74 (t, 2H), 2.61 (t, 2H), 1.74 (m, 2H); MS 170 (M+1).

### Preparation 13

### (3-Formyl-phenyl)-acetic acid., methyl ester

### Step A

(3-Cyano-phenyl)-acetic acid methyl ester. Nitrogen was bubbled through a mixture of (3-bromo-phenyl)-acetic acid methyl ester (22.85 g, 99.78 mmol), Zn(CN)₂ (7.25 g, 61.75 mmol), and DMF (0.100 mL) for about 5 minutes followed by addition of tetrakistriphenylphosphine(0) palladium (4.60 g, 3.98 mmol): The mixture was heated for 3 h at 80° C. and was cooled to room temperature. Aqueous 2N NH₄OH was added and the product was extracted into EtOAc (3×). The organic solution was washed with 2N NH₄OH (2×) followed by brine (2×). The organic solution was dried (MgSO₄), filtered, and concentrated in vacuo. Purification by flash chromatography (6:1 hexanes:EtOAc) provided the title compound of Step A as an oil (15.19 g). ¹H NMR (400 MHz, CDCl₃) δ 7.57-7.41 (m, 4H), 3.706 (s, 3H), 3.703 (s, 2H).

### Step B

(3-Formyl-phenyl)-acetic acid methyl ester. A mixture of (3-cyano-phenyl)-acetic acid methyl, ester prepared of Preparation 13, Step A (1.56 g, 8.91 mmol), aluminum-nickel alloy (1.63 g) and 75% formic acid (25, mL) was heated at reflux for 1.75 h. The mixture was cooled to room temperature and the solids were removed by filtration through Celite® with the aid of boiling EtOH. Water was added, and the aqueous solution was washed with CH₂Cl₂ (3×). Aqueous saturated NaHCO₃ was carefully added to the organic solution until the pH was about 8-9. The organic solution was washed with brine, dried over MgSO₄, and concentrated. Purification by flash chromatography (5:1 hexanes:EtOAc) provided the title compound as a clear and colorless oil (870 mg). ¹H NMR (400 MHz, CDCl₃) δ 9.98 (s, 1H), 7.77 (m, 2H), 7.55-7.46 (m, 2H), 3.68 (s, 5H).

### Preparation 14

(3-((Pyridine-3-sulfonylamino)-methyl)-phenyl)-acetic acid methyl ester. To a solution of (3-aminomethyl-phenyl)-acetic acid methyl ester hydrochloride (from Preparation 18, 0.56 g) and diisopropylamine (2.2 mL) in 10 mL dichloromethane was added pyridine-3-sulfonyl chloride (from Preparation 2, 0.601 g, 2.83 mmol) and the reaction was stirred at room temperature for 16 h. Aqueous 1N HCl was added and the solution was washed with CH₂Cl₂. The organic solution was washed with saturated NaHCO₃, dried over MgSO₄, filtered and concentrated in vacuo to afford the title compound. Purification via flash chromatography on silica gel (2:1 hexanes:EtOAc) afforded the title compound as a white solid. ¹H NMR (400 MHz, CDCl₃) δ 8.91 (s, 1H), 8.71 (d, 1H), 8.04 (d, 1H), 7.37 (m, 1H),7.05-7.24 (m, 4H), 5.87 (bs, 1H), 4.14 (s, 2H), 3.62 (s, 3H), 3.52 (s, 2H).

### Preparation 15

### Method A

4-Butylbenzylamine. A solution of 4-butylbenzonitrile (3.63 g, 22.8 mmol) in THF (10 mL) was placed in a three-neck round bottom flask equipped with a Vigreux column and short-path distillation head. The solution was heated to reflux and BH₃-methyl sulfide complex (2.0 M in THF, 15 mL, 30 mmol) was added dropwise over 15 minutes. Methyl sulfide was distilled off from the reaction mixture over 1 h and the solution was cooled to room temperature. Aqueous HCl (6N, 25 mL) was added slowly via an addition funnel and the mixture was heated at reflux for 30 minutes. The reaction was cooled to 0° C. and NaOH (7.0 g) was added portionwise. The aqueous solution was washed with EtOAc (3×) and the organic solution was dried (MgSO₄), filtered, and concentrated to provide the title compound of Method A (4.01 g). ¹H NMR (400 MHz, CDCl₃) δ 7.34 (m, 2H), 7.24 (m, 2H), 4.04 (s, 2H), 2.62 (t, 2H), 1.58 (m, 2H), 1.34 (m, 2H), 0.92 (t, 3H).

### Method B

4-Butylbenzylamine hydrochloride. A solution of 4-butylbenzonitrile (30.09 g) in EtOH (380 mL) and HCl (4N in dioxane, 50 mL, 200 mmol) was hydrogenated at 50 psi on a Parr shaker in the presence of 10% palladium on carbon (6.09 g). The catalyst was removed via filtration through Celite® and the solution was concentrated in vacuo. The residue was suspended in Et₂O and filtered to provide 4-butylbenzylamine hydrochloride as an off-white solid (32.47 g). ¹H NMR (400 MHz, CD₃OD) δ 7.33 (d, 2H), 7.22 (d, 2H), 4.04 (s, 2H), 2.60 (t, 2H), 1.56 (m, 2H), 1.31 (m, 2H), 0.89 (t, 3H).

Using the appropriate starting materials, the compounds of Preparations 16-18 were prepared in a manner analogous to the method of Preparation 15.

### Preparation 16

2-(3.5-Dichloro-phenoxy)-ethylamine. The title compound was prepared following Method A of Preparation 15.

### Preparation 17

2-(3-Chloro-phenoxy)-ethylamine. The title compound was prepared following Method A of Preparation 15.

### Preparation 18

(3-Aminomethyl-phenyl)-acetic acid methyl ester hydrochloride. The title compound was prepared from (3-cyano-phenyl)-acetic acid methyl ester (from Preparation 13, Step A) using the procedure described for Preparation 15, Method B except the hydrogenation was performed in MeOH. The catalyst was removed via filtration and the organic solution was concentrated in vacuo. The resulting solid was stirred in EtOAc and filtered to provide the title compound as a white solid. ¹H NMR (400 MHz, CD₃OD) L 7.42-7.32 (m, 4H), 4.09 (s, 2H), 3.69 (s, 2H), 3.67 (s, 3H); MS 180 (M+1).

### Preparation 19

### trans-1-(3-Bromo-propenyl)-3.5-dichloro-benzene

### Step A

1-(3,5-Dichloro-phenyl)-prop-2-en-1-ol. A solution of 3,5-dichlorobenzaldehyde (7.5 g, 43 mmol) in THF (75 mL) was cooled to 0° C. and vinylmagnesium bromide (1M in THF, 48 mL, 48 mmol) was added dropwise. The reaction was warmed to room temperature and was stirred for 16 h. Aqueous HCl (1N) and EtOAc were added. The aqueous solution was washed with EtOAc and the organic solution was dried (MgSO₄), filtered, and concentrated. The residue was used in the next step without further purification.

### Step B

The residue prepared in Step A was dissolved in Et₂O and HBr gas was slowly bubbled into the solution for about 15 minutes. The reaction was stirred at room temperature for 24 h and water and EtOAc were added. The aqueous solution was extracted with EtOAc and the organic solution was dried (MgSO₄), filtered, and concentrated. Purification by flash chromatography (hexanes) provided the title compound of Preparation 19 (6.91 g). ¹H NMR (400 MHz, CDCl₃) δ 7.24 (s, 3H), 6.53 (d, 1H), 6.40 (m, 1H), 4.10 (m, 2H).

### Preparation 20

### (3-Aminomethyl-phenoxy)-acetic acid tert-butyl ester

### Step A

(3-Formyl-phenoxy)-acetic acid tert-butyl ester. To a solution of 3-hydroxybenzaldehyde (5.00 g, 40.9 mmol) in DMF (40 mL) was added 1 M potassium tert-butoxide in tert-butanol (40.9 mL, 40.9 mmol). The reaction was stirred for 2 minutes and tert-butyl bromoacetate (6.61 mL, 40.9 mmol) was added. The reaction was stirred for 1 hour and was quenched with 200 mL water. The product was extracted into EtOAc and the organic solution was washed with water, dried over MgSO₄, filtered, and concentrated in vacuo. Purification via flash chromatography on silica gel (9:1 hexanes:EtOAc) afforded the title compound of Step A as a clear oil (3.53 g). ¹H NMR (400 MHz, CDCl₃) δ 9.94 (s, 1H), 7.48 (m, 2H), 7.32 (s, 1H), 7.21 (m, 1H), 4.56 (s, 2H), 1.45 (s, 9H).

### Step B

(3-(Hydroxyimino-methyl)-phenoxy)-acetic acid tert-butyl ester. To a solution of (3-formyl-phenoxy)-acetic acid tert-butyl ester prepared of Preparation 20, Step A (2.05 g, 8.68 mmol) in MeOH (30 mL) was added NH₂OH.HCl (0.66 g, 9.54 mmol) and pyridine (3.5 mL, 43.4 mmol) and the reaction was stirred for 2 hours. The MeOH was removed in vacuo and the residue was diluted with EtOAc and 1N HCl. The layers were separated and the aqueous solution was washed with EtOAc. The combined organic layers were dried over MgSO₄, filtered and concentrated in vacuo to afford the title compound of Step B (1.99 g). ¹H NMR (400 MHz, CDCl₃) δ 8.07 (s, 1H), 7.23-7.28 (m, 2H), 7.12 (m, 1H), 6.93 (d, 1H), 4.51 (s, 2H), 1.46 (s, 9H).

### Step C

(3-Aminomethyl-phenoxy)-acetic acid tert-butyl ester. To a solution of (3-(hydroxyimino-methyl)-phenoxy)-acetic acid tert-butyl ester prepared of Preparation 20, Step B (2.25 g, 5.96 mmol) in EtOH (10 mL) was added Raney Nickel (about 1 g, washed with water followed by EtOH) in 100 mL EtOH. Additional EtOH (90 mL) was required for the transfer. Ammonium hydroxide (10 mL) was added and the mixture was shaken under 45 psi of H₂ for 4 hours. The catalyst was removed via filtration through Celite® and the solution was concentrated to a clear oil. Purification via flash chromatography on silica gel (96.5/3.5/0.1 to 9/1/0.1 CH₂Cl₂/MeOH/NH₄OH) afforded the title compound as a yellow oil. ¹H NMR (400 MHz, CDCl₃) δ 7.23 (m, 1H), 6.92 (m, 2H), 6.72 (d, 1H), 4.50 (s, 2H), 3.82 (s, 2H), 1.96 (m, 2H), 1.46 (s, 9H); MS 238 (M+1).

### Preparation 21

### 4-Pyrimidin-2-yl-benzaldehyde

A solution of 2-bromopyrimidine (1.00 g, 6.3 mmol) and tetrakistriphenylphosphine(0) palladium (0.218 g, 0.189 mmol) in ethylene glycol dimethyl ether (30 mL) was stirred at room temperature for 10 minutes. A solution of 4-formylbenzene boronic acid (1.14 g, 7.61 mmol) and sodium bicarbonate (1.58 g, 18.9 mmol) in 15 mL water was added and the reaction was heated at reflux for 16 h. The mixture was diluted with water and CH₂Cl₂. The layers were separated, and the aqueous solution was washed with CH₂Cl₂. The combined organic layers were dried over MgSO₄, filtered, and concentrated in vacuo. The residue was purified via flash chromatography on silica gel (10% to 30% hexanes in EtOAc) to afford the title compound (0.979 g). ¹H NMR (400 MHz, CDCl₃) δ 10.11 (s, 1H), 8.83 (s, 2H), 8.82 (s, 1H), 7.98 (s, 2H), 7.23 (s, 2H).

### Preparations 22-27

Preparations 22-27 were prepared from the appropriate starting materials in a manner analogous to the method of Preparation 21.

### Preparation 22

### 4-Pyridin-2-yl-benzaldehyde

¹H NMR (400 MHz, CDCl₃) δ 10.09 (s, 1H), 8.72 (s, 1H),8.16 (s, 2H), 7.95 (s, 2H), 7.79 (s, 2H), 7.29 (m, 1H); MS 184 (M+1).

### Preparation 23

### 4-Pyridin-3-yl-benzaldehyde

¹H NMR (400 MHz, CDCl₃) δ 10.04 (s, 1H), 8.88 (s, 1H),8.64 (s, 1H), 7.97 (s, 2H), 7.91 (m, 1H), 7.75 (m, 2H), 7.39 (m, 1H); MS 184 (M+1).

### Preparation 24

### 4-Pyridin-4-yl-benzaldehyde

¹H NMR (400 MHz, CDCl₃) δ 10.03 (s, 1H), 8.70 (s, 2H),7.99 (s, 2H), 7.79(s, 2H), 7.52 (s, 2H); MS 184 (M+1).

### Preparation 25

### 4-Thiazol-2-yl-benzaldehyde

MS 189 (M+).

### Preparation 26

### 4-Pyrimidin-5-yl-benzaldehyde

¹H NMR (400 MHz, CDCl₃) δ 10.03 (s, 1H), 9.26 (s, 1H), 9.00 (s, 2H), 8.03 (m, 2H), 7.76 (m, 2H).

### Preparation 27

### 4-Pyrazin-2-yl-benzaldehyde

¹H NMR (400 MHz, CDCl₃) δ 10.03 (s, 1H), 9.10 (s, 1H), 8.69 (s, 1H), 8.59 (s, 1H), 8.21 (d, 2H), 8.03 (d, 2H).

### Preparation 28

1-(2-Bromo-ethoxy)-3,5-dichloro-benzene. To a solution of NaOH (2.45 g, 61.3 mmol) in water (20 mL) was added 3,5-dichlorophenol (5 g, 30.7 mmol). The solution was heated at reflux for 1 h and was cooled to room temperature. 1,2-Dibromoethane (11.52 g, 61.3 mmol) was added and the reaction was heated at reflux for 24 h. The cooled solution was diluted with EtOAc and the organic solution was washed sequentially with HCl (1N, 1×), water (1×), and brine (1×). The organic solution was dried (MgSO₄), filtered, and concentrated. Purification by flash chromatography (hexanes to 5% EtOAc in hexanes) provided the title compound (3.79 g). ¹H NMR (400 MHz, CDCl₃) δ 6.98 (m, 1H), 6.82 (m, 2H), 4.25 (t, 2H), 3.61 (t, 2H).

### Preparation 29

1-(2-Bromo-ethoxy)-3-chlorobenzene. The compound of Preparation 29 was prepared from the appropriate starting materials in a manner analogous to the method of Preparation 28.

### Preparation 30

### 4-[(1-Acetyloxy)-hexyl]-benzyl bromide

### Step A: Griqnard Reaction and Protection

4-((1-Acetyloxy)-hexyl)-toluene. Pentylmagnesium bromide (2.0 M in Et₂O, 25 mL, 50 mmol) was added slowly to p-tolylbenzaldehyde (5.0 mL, 42.4 mmol) in THF (50 mL) at 0° C. The reaction was warmed to room temperature and was stirred for 3 h. Aqueous 1 N HCl was added and the aqueous solution was extracted with EtOAc. The organic solution was washed with brine, dried over MgSO₄, filtered, and concentrated. The residue was dissolved in pyridine (35 mL) and Ac₂O (10 mL) was added. The reaction was stirred for 24 h and was diluted with water. The product was extracted into EtOAc (3×) and the organic solution was washed with 1N HCl followed by brine, dried over MgSO₄, filtered, and concentrated. The product was purified by flash chromatography (10% EtOAc/hexanes) to afford 4-((1-acetyloxy)-hexyl)-toluene (2.082 g). ¹H NMR (400 MHz, CDCl₃) δ 7.12-7.28 (m, 4H), 5.69 (t, 1H), 2.33 (s, 3H), 2.04 (s, 3H), 1.88 (m, 1H), 1.74 (m, 1H), 1.27 (m, 6H), 0.86 (m, 3H); MS 252 (M+18).

### Step B: Benzylic Bromination

A mixture of 4-[(1-acetyloxy)-hexyl]-toluene prepared of Preparation 30, Step A (2.082 g, 8.89 mmol), N-bromosuccinimide (1.58 g, 8.89 mmol), and catalytic 2,2′-azobisisobutyronitrile in carbon tetrachloride (30 mL) was heated at reflux for 2 h. The reaction was cooled and was washed with aqueous NaHCO₃ (saturated), dried over MgSO₄, filtered, and concentrated. The product was purified by flash chromatography (5% EtOAc/hexanes) to afford the title compound of Preparation 30 (2.67 g). ¹H NMR (400 MHz, CDCl₃) δ 7.34-7.40 (m, 4H), 5.70 (t, 1H), 4.47 (s, 2H), 2.06 (s, 3H), 1.86 (m, 1H), 1.73 (m, 1H), 1.27 (m, 6H), 0.85 (m, 3H).

### Preparation 31

1-Methyl-1H-indole-2-carbaldehyde. The title compound can be prepared using the method described by Comins and coworkers in J. Org. Chem., 52, 1, 104-9, 1987.

### Preparation 32

5-Phenyl-furan-2-carbaldehyde. The title compound can be prepared using the method described by D'Auria and coworkers in Heterocycles, 24, 6, 1575-1578, 1986.

### Preparation 33

4-Phenethylsulfanyl-benzaldehyde. The title compound can be prepared using the method described by Clark and coworkers in EP 332331.

### Preparation 34

3-Hydroxy-4-propoxy-benzaldehyde. The title compound can be prepared using the method described by Beke in Acta Chim. Acad. Sci. Hung., 14, 325-8, 1958.

### Preparation 35

4-Formyl-N-methyl-benzenesulfonamide. The title compound can be prepared using the method described by Koetschet in Helv. Chim. Acta., 12, 682, 1929.

### Preparation 36

4-Chloro-thiophene-2-carbaldehyde. The title compound can be prepared using the method described by Raggon and coworkers in Org. Prep. Proced. Int.; EN, 27, 2, 233-6, 1995.

### Preparation 37

4-Cyclohexyl-benzylamine. The title compound can be prepared using the method described by Meglio and coworkers in Farmaco Ed. Sci.; IT; 35, 3, 191-202, 1980.

### Preparation 38

4-Imidazol-1-yl-benzaldehyde. The title compound can be prepared using the method described by Sircar and coworkers in J. Med. Chem. 30, 6, 1023-9, 1987.

### Preparation 39

4-(2-Oxo-pyrrolidin-1-yl)-benzaldehyde. The title compound can be prepared using the method described by Kukalenko in Chem. Heterocycl. Compd. (Engl. Transl.), 8, 43, 1972.

### Preparation 40

2-(3-Chloro-phenylsulfanyl)-ethylamine. The title compound can be prepared using the method described by Elz and coworkers in Fed. Rep. Ger. Sci. Pharm., 56, 4, 229-234, 1988.

### Preparation 41

Prop-2-vnyl-carbamic acid t-butyl ester. The title compound can be prepared using the method described in J. Chem. Soc. Perkin Trans. 1, 1985, 2201-2208. Unless otherwise specified, all reactions were performed under an inert atmosphere such as nitrogen (N₂).

NMR spectra were recorded on a Varian XL-300 (Varian Co., Palo Alto, Calif.), a Bruker AM-300 spectrometer (Bruker Co., Billerica, Mass.) or a Varian Unity 400 at about 23° C. at 300 or 400 MHz for proton and 75.4 MHz for carbon nuclei. Chemical shifts are expressed in parts per million downfield from trimethylsilane. The peak shapes are denoted as follows: s, singlet; d, doublet; t, triplet, q, quartet; m, multiplet; bs=broad singlet. Resonances designated as exchangeable did not appear in a separate NMR experiment where the sample was shaken with several drops of D₂O in the same solvent. Atmospheric pressure chemical ionization (APCI) mass spectra were obtained on a Fisons Platform II Spectrometer. Chemical ionization mass spectra were obtained on a Hewlett-Packard 5989 instrument (Hewlett-Packard Co., Palo Alto, Calif.) (ammonia ionization, PBMS). Where the intensity of chlorine or bromine-containing ions are described the expected intensity ratio was observed (approximately 3:1 for ³⁵Cl/³⁷Cl-containing ions) and 1:1 for ⁷⁹Br/⁸¹Br-containing ions) and the intensity of only the lower mass ion is given.

Column chromatography was performed with either Baker Silica Gel (40 μm) (J. T. Baker, Phillipsburg, N.J.) or Silica Gel 60 (EM Sciences, Gibbstown, N.J.) in glass columns under low nitrogen pressure. Radial Chromatography was performed using a Chromatotron® (model 7924T, Harrison Research). Medium pressure chromatography was performed on a Flash 40 Biotage System (Biotage Inc, Dyax Corp., Charlottesville, Va.). Unless otherwise specified, reagents were used as obtained from commercial sources. Dimethylformamide, 2-propanol, acetonitrile, methanol, tetrahydrofuran, and dichloromethane, when used as reaction solvents, were the anhydrous grade supplied by Aldrich Chemical Company (Milwaukee, Wis.). The terms “concentrated” and “coevaporated” refer to removal of solvent at water aspirator pressure on a rotary evaporator with a bath temperature of less than 45° C. Reactions conducted at “0-2020 C.” or “0-25° C.” were conducted with initial cooling of the vessel in an insulated ice bath which was allowed to warm to room temperature over several hours. The abbreviation “min” and “h” stand for “minutes” and “hours” respectively. DTT means dithiothreitol. DMSO means dimethyl sulfoxide. EDTA means ethylenediamine tetraacetic acid.

Some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

### Biological Assays

### Determination of cAMP Elevation in 293-S Cell Lines Stably Overexpressing Recombinant Human EP2 and EP₄ Receptors

cDNAs representing the complete open reading frames of the human EP₂ and EP₄ receptors are generated by reverse transcriptase polymerase chain reaction using oligonucleotide primers based on published sequences (1, 2) and RNA from primary human kidney cells (EP₂) or primary human lung cells (EP₄) as templates. cDNAs are cloned into the multiple cloning site of pcDNA3 (Invitrogen Corporation, 3985B Sorrento Valley Blvd., San Diego, Calif. 92121) and used to transfect 293-S human embryonic kidney cells via calcium phosphate co-precipitation. G418-resistant colonies are expanded and tested for specific [³H]PGE₂ binding. Transfectants demonstrating high levels of specific [³H]PGE₂ binding are further characterized by Scatchard analysis to determine Bmax and Kds for PGE₂. The lines selected for compound screening have approximately 338,400 receptors per cell and a Kd=12 nM for PGE₂ (EP₂), and approximately 256,400 receptors per cell and a Kd=2.9 nM for PGE₂ (EP₄). Constituitive expression of both receptors in parental 293-S cells is negligible. Cells are maintained in RPMI supplemented with fetal bovine serum (10% final) and G418 (700 ug/ml final).

cAMP responses in the 293-S/EP₂ and 293-S/EP₄ lines are determined by detaching cells from culture flasks in 1 ml of Ca++ and Mg++ deficient PBS via vigorous pounding, adding serum-free RPMI to a final concentration of 1×10⁶ cells/ml, and adding 3-isobutyl-1-methylxanthine (IBMX) to a final concentration of 1 mM. One milliliter of cell suspension is immediately aliquoted into individual 2 ml screwcap microcentrifuge and incubated for 10 minutes, uncovered, at 37° C., 5% CO₂, 95% relative humdity. The compound to be tested is then added to cells at 1:100 dilutions such that final DMSO or ethanol concentrations is 1%. Immediately after adding compound, the tubes are covered, mixed by inverting two times, and incubated at 37° C. for 12 minutes. Samples are then lysed by incubation at 100° C. for 10 minutes and immediately cooled on ice for 5 minutes. Cellular debris is pelleted by centrifugation at 1000×g for 5 minutes, and cleared lysates are transferred to fresh tubes. cAMP concentrations are determined using a commercially available cAMP radioimmunoassay. kit RIA (NEK-033, DuPont/NEN Research Products, 549 Albany St., Boston, Mass. 02118) after diluting cleared lysates 1:10 in cAMP RIA assay buffer (included in kit). Typically, one treats cells with 6-8 concentrations of the compound to be tested in 1 log increments. EC50 calculations are performed on a calculator using linear regression analysis on the linear portion of the dose response curves.

### REFERENCES

1. Regan, J. W. Bailey, T. J. Pepperl, D. J. Pierce, K. L. Bogardus, A. M. Donello, J. E. Fairbairn, C. E. Kedzie, K. M. Woodward, D. F. and Gil, D. W. 1994 Cloning of a Novel Human Prostaglandin Receptor with Characteristics of the Pharmaclogically, Defined EP₂ Subtype. Mol. Pharmacology 46:213-220.
2. Bastien, L., Sawyer, N., Grygorczyk, R., Metters, K., and Adam, M. 1994 Cloning, Functional Expression, and Characterization of the Human Prostaglandin E2 Receptor EP2 Subtype. J. Biol. Chem. Vol 269, 16:11873-11877.

### Assay for Binding to Prostaglandin E₂ Receptors

Membrane Preparation: All operations are performed at 4 IC. Transfected cells expressing prostaglandin E₂ type 1 receptors (EP₁), type 2 (EP₂), type 3 (EP₃) or type 4 (EP₄) receptors are harvested and suspended to 2 million cells per ml in Buffer A [50 mM Tris-HCl (pH 7.4), 10 mM MgCl₂, 1 mM EDTA, 1 mM Pefabloc peptide, (Boehringer Mannheim Corp., Indianapolis, Ind.), 10 uM Phosporamidon peptide, (Sigma, St. Louis, Mo.), 1 uM pepstatin A peptide, (Sigma, St. Louis, Mo.), 10 uM elastatinal peptide, (Sigma, St. Louis, Mo.), 100 uM antipain peptide, (Sigma, St. Louis, Mo.)]. The cells are lysed by sonification with a Branson Sonifier (Model #250, Branson Ultrasonics Corporation, Danbury, Conn.) in 2 fifteen second bursts. Unlysed cells and debris are removed by centrifugation, at 100×g for 10 min. Membranes are then harvested by centrifugation at 45,000×g for 30 minutes. Pelleted membranes are resuspended to 3-10 mg protein per ml, protein concentration being determined of the method of Bradford [Bradford, M., Anal. Biochem., 72, 248 (1976)]. Resuspended membranes are then stored frozen at −80° C. until use.

Binding Assay: Frozen membranes prepared as above are thawed and diluted to 1 mg protein per ml in Buffer A above. One volume of membrane preparation is combined with 0.05 volume test compound or buffer and one volume of 3 nM ³H-prostaglandin E₂ (#TRK 431, Amersham, Arlington Heights, Ill.) in Buffer A. The mixture (205 μL total volume) is incubated for 1 hour at 25° C. The membranes are then recovered by filtration through type GF/C glass fiber filters (#1205-401, Wallac, Gaithersburg, Md.) using a Tomtec harvester (Model Mach 11/96, Tomtec, Orange, Conn.). The membranes with bound ³H-prostaglandin E₂ are trapped by the filter, while the buffer and unbound ³H-prostaglandin E₂ pass through the filter into waste. Each sample is then washed 3 times with 3 ml of [50 mM Tris-HCl (pH 7.4), 10 mM MgCl₂, 1 mM EDTA]. The filters are then dried by heating in a microwave oven. To determine the amount of ³H-prostaglandin bound to the membranes, the dried filters are placed into plastic bags with scintillation fluid and counted in a LKB 1205 Betaplate reader (Wallac, Gaithersburg, Md.). IC50s are determined from the concentration of test compound required to displace 50% of the specifically bound ³H—, prostaglandin E₂.

The full length EP, receptor is made as disclosed in Funk et al., Journal of Biological Chemistry, 1993, 268, 26767-26772. The full length EP₂ receptor is made as disclosed in Regan et al., Molecular Pharmacology, 1994, 46, 213-220. The full length EP₃ receptor is made as disclosed in Regan et al., British Journal of Pharmacology, 1994, 112, 377-385. The full length EP₄ receptor is made as disclosed in Bastien, Journal of Biological Chemistry, 1994, 269, 11873-11877. These full length receptors are used to prepare 293S cells expressing the EP₁, EP₂, EP₃ and EP₄ receptors.

293S cells expressing either the human EP₁, EP₂, EP₃ or EP₄ prostaglandin E₂ receptors are generated according to methods known to those skilled in the art. Typically, PCR (polymerase chain reaction) primers corresponding to the 5′ and 3′ ends of the published full length receptor are made according to the well known methods disclosed above and are used in an RT-PCR reaction using the total RNA from human kidney (for EP₁), human lung (for EP₂), human lung (for EP₃) or human lymphocytes (for EP₄) as a source. PCR products are cloned by the TA overhang method into pCR2.1 (Invitrogen, Carlsbad, Calif.) and identity of the cloned receptor is confirmed by DNA sequencing.

293S cells (Mayo, Dept. of Biochemistry, Northwestern Univ.) are transfected with the cloned receptor in pcDNA3 by electroporation. Stable cell lines expressing the receptor are established following selection of transfected cells with G418.

Clonal cell lines expressing the maximal number of receptors are chosen following a whole cell ³H-PGE₂ binding assay using unlabeled PGE₂ as a competitor.

| Example | |
|---|---|
| Number | EP₂ (nM) |
| 1 | 395 |
| 1a | 2200 |
| 1b | 160 |
| 1c | 24 |
| 1d | 885 |
| 1e | 760 |
| 1f | 52 |
| 1g | 109 |
| 1h | 745 |
| 1i | 72 |
| 1j | 385 |
| 1k | 1755 |
| 1l | 600 |
| 1m | 245 |
| 1n | 140 |
| 1o | 550 |
| 1p | 44 |
| 1q | 39 |
| 1r | 308 |
| 1s | 165 |
| 1t | 8 |
| 1u | 7 |
| 1v | 9 |
| 1w | 24 |
| 1x | 160 |
| 1y | 13 |
| 1z | 19 |
| 1aa | 335 |
| 1ab | 110 |
| 1ac | 335 |
| 1ad | 195 |
| 1ae | 121 |
| 1af | 106.5 |
| 1ag | 169 |
| 1ah | 180.5 |
| 1ai | 125 |
| 1aj | 25.5 |
| 1ak | 6.19 |
| 1al | 17 |
| 1an | 94 |
| 2 | 45 |
| 2a | 37 |
| 2b | 385 |
| 3 | 650 |
| 3a | 440 |
| 3b | 7.83 |
| 4 | 225 |
| 4a | 795 |
| 4b | 440 |
| 4c | 885 |
| 4d | 175 |
| 4e | 410 |
| 4f | 705 |
| 4g | 720 |
| 4h | 210 |
| 5 | 980 |
| 5a | 355 |
| 5b | 640 |
| 6 | 315 |
| 6a | 325 |
| 6c | 300 |
| 6d | 170 |
| 6e | 325 |
| 6f | 490 |
| 6g | 350 |
| 6h | 140 |
| 6i | 270 |
| 6j | 375 |
| 7a | 400 |
| 7b | 140 |
| 7c | 260 |
| 7d | 52 |
| 7f | 344 |
| 7g | 412 |
| 7h | 172 |
| 8 | 71 |
| 8a | 180 |
| 8b | 266 |
| 9 | 17 |
| 9a | 84.5 |
| 9b | 94.5 |
| 9c | 490 |
| 9d | 202.5 |
| 11a | 8.2 |
| 11b | 12.75 |
| 11c | 13.2 |
| 11d | 45 |
| 11e | 6.3 |
| 11f | 25.68 |
| 11g | 25.04 |
| 11h | 7.5 |
| 11l | 89 |
| 11m | 10 |
| 11n | 10 |
| 11o | 10 |
| 11p | 10 |
| 11q | 10 |
| 11r | 10 |
| 11s | 10 |
| 11t | 10 |
| 11u | 10.5 |
| 11v | 10 |
| 11w | 36.5 |
| 11x | 63.5 |
| 11y | 10 |
| 11z | 13 |
| 12a | 65 |
| 12b | 14.3 |
| 12c | 25 |
| 12d | 515 |
| 12e | 118.5 |
| 12f | 10 |
| 12g | 10 |
| 12h | 10 |
| 12i | 16 |
| 12k | 31 |
| 12m | 38 |
| 12n | 10 |
| 12o | 10 |
| 12p | 29 |
| 12q | 10 |
| 12r | 10 |
| 12s | 10 |
| 12t | 57 |
| 12u | 63 |
| 12v | 10 |
| 12w | 47 |
| 12x | 10 |
| 12y | 55 |
| 12z | 200 |
| 13a | 10 |
| 13b | 22 |
| 13c | 10 |
| 13d | 10 |
| 13e | 10 |
| 13f | 10 |
| 13g | 340 |
| 13h | 10 |
| 13i | 10.4 |
| 13j | 26 |
| 13k | 99 |
| 13l | 47.5 |
| 13m | 155 |
| 130 | 10 |
| 13r | 10 |
| 13s | 10 |
| 13t | 230 |
| 13u | 7.99 |
| 13v | 25.83 |
| 13w | 12 |
| 13x | 15.5 |
| 13y | 10 |
| 13z | 24.5 |
| 14a | 13.5 |
| 14b | 10 |
| 14c | 68 |
| 14d | 10 |
| 14e | 13 |
| 15a | 10 |
| 15b | 10 |
| 15c | 35 |
| 15d | 10 |
| 15e | 39 |
| 15f | 10 |
| 15g | 10 |
| 16a | 110 |
| 16b | 215 |

1. A method of treating pulmonary hypertension, the method comprising administering to a patient in need thereof a therapeutically effective amount of an EP₂ selective receptor agonist.

2. The method of claim 1 wherein the EP₂ selective receptor agonist is a compound of Formula I 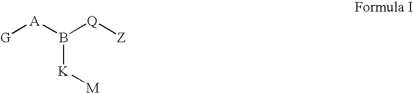
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of, W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic, ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are, each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N, N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

3. The method of claim 2 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

4. A method of facilitating joint fusion, the method comprising administering to a patient in need thereof a therapeutically effective amount of an EP₂ selective receptor agonist.

5. The method of claim 4 wherein the EP₂ selective receptor agonist is a compound of Formula I 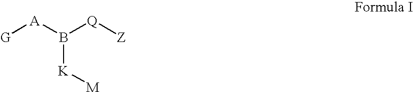
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, wherein containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

6. The method of claim 5 wherein the EP₂ selective receptor agonist-is (3-(((4-tert-butyl-benzyl)-(pyridine-3-sulfonyl)-amino)-methyl)-phenoxy)-acetic acid or a pharmaceutically acceptable salt thereof.

7. A method of facilitating tendon or ligament repair, the method comprising administering to a patient in need thereof a therapeutically effective amount of an EP₂ selective receptor agonist.

8. The method of claim 7 wherein the EP₂ selective receptor agonist is a compound of Formula I 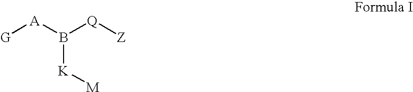
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene; R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a b, fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and. R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or, mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R₂, R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

9. The method of claim 8 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

10. A method of reducing the occurrence of secondary fracture, the method comprising administering to a patient in need thereof a therapeutically effective amount of an EP₂ selective receptor agonist.

11. The method of claim 10 wherein the EP₂ selective receptor agonist is a compound of Formula I 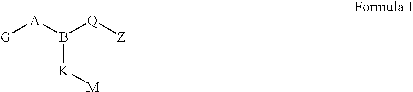
or a prodrug thereof, or a pharmaceutically acceptable salt thereof, wherein
A is SO₂ or CO;
G is Ar, Ar¹-V-Ar, Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the, amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-Q₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

12. The method of claim 11 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

13. A method of treating avascular necrosis, the method comprising administering to a patient in need thereof a therapeutically effective amount of an EP₂ selective receptor agonist.

14. The method of claim 13 wherein the EP₂ selective receptor agonist is a compound of Formula I 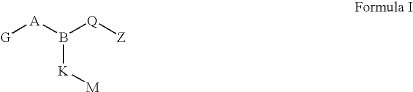
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
Bis N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C—.C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar ⁵, —Ar⁴—S—Ar ⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully, saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N , N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully, saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′
—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, Oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

15. The method of claim 14 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

16. A method of facilitating cartilage repair, the method comprising administering to a patient in need thereof a therapeutically effective amount of an EP₂ selective receptor agonist.

17. The method of claim 16 wherein the EP₂ selective receptor agonist is a compound of Formula I 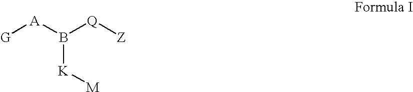
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro, or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of, W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₆-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-Q₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴-V-Ar⁵, —Ar⁴—S—Ar⁵, —Ar⁴—SO—Ar⁵, —Ar⁴—S₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N, N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-Q₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(c₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

18. The method of claim 17 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

19. A method of facilitating bone healing after limb transplantation, the method comprising administering to a patient in need thereof a therapeutically effective amount of an EP₂ selective receptor agonist.

20. The method of claim 19 wherein the EP₂ selective receptor agonist is a compound of Formula I 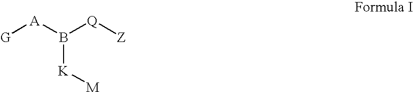
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹¹, R², R³, R⁴, R⁵, R¹¹, R R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

21. The method of claim 20 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

22. A method of facilitating liver regeneration, the method comprising administering to a patient in need thereof a therapeutically effective amount of an EP₂ selective receptor agonist.

23. The method of claim 22 wherein the EP₂ selective receptor agonist is a compound of Formula I 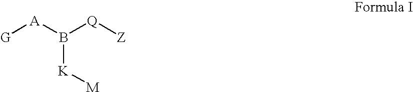
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene; R¹R²-amino, oxy(C₁-C₆)alkylene, amino substituted with Ar, or amino substituted with Ar(C₁-C₄)alkylene and R¹¹, wherein R¹¹ is H or. (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said, alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl, —(C₁-C₄)alkylene-ethenylene-(C₁-C₄)alkylene-, said alkylenes and said ethenylene each optionally substituted with up to four substituents each independently selected from fluoro or (C₁-C₄)alkyl, —(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

24. The method of claim 23 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

25. A method of facilitating wound healing, the method comprising administering to a patient in need thereof a therapeutically effective amount of an EP₂ selective receptor agonist.

26. The method of claim 25 wherein the EP₂ selective receptor agonist is a compound of Formula I 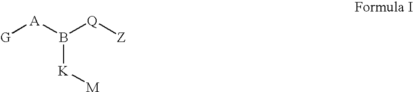
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the, amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl, —(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl, —(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and, said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³?-Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-CB)alkylene; and M is Ar³ or Ar⁴—Ar⁵, then A is sulfonyl.

27. The method of claim 26 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

28. A method of reducing the occurence of gastric ulceration, the method comprising administering to a patient in need thereof a therapeutically effective amount of an EP₂ selective receptor agonist.

29. The method of claim 28 wherein the EP₂ selective receptor agonist is a compound of Formula I, 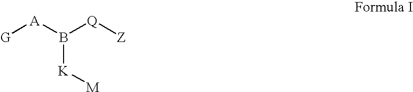
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl, —(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X-(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen., sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C, —C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N; N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

30. The method of claim 29 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

31. A method of treating hypertension, the method comprising administering to a patient in need thereof a therapeutically effective amount of an EP₂ selective receptor agonist.

32. The method of claim 31 wherein the EP₂ selective receptor agonist is a compound of Formula I 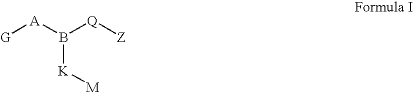
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
Bis N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro, or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴-V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are, each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (Q₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

33. The method of claim 32 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

34. A method of facilitating the growth of tooth enamel, or finger or toe nails, the method comprising administering to a patient in need thereof a therapeutically effective amount of an EP₂ selective receptor agonist.

35. The method of claim 34 wherein the EP₂ selective receptor agonist is a compound of Formula I 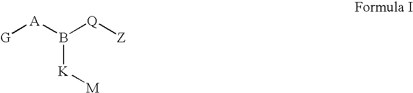
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar¹, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
Bis N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl, 7(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X-(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or, tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-° C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′
—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹,R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

36. The method of claim 35 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

37. A method of treating glaucoma, the method comprising administering to a patient in need thereof a therapeutically effective amount of an EP₂ selective receptor agonist.

38. The method of claim 37 wherein the EP₂ selective receptor agonist is a compound of Formula I 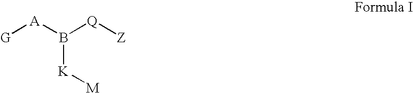
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or, two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N, N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵ R¹¹, R³¹, R⁴¹ and R⁵¹, when containing an alkyl, alkylene, alkenylene or alkynylene moiety, are optionally mono-, di- or tri-substituted on carbon independently, with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

39. The method of claim 38 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

40. A method of treating ocular hypertension, the method comprising administering to a patient in need thereof a therapeutically effective amount of an EP₂ selective receptor agonist.

41. The method of claim 40 wherein the EP₂ selective receptor agonist is a compound of Formula I 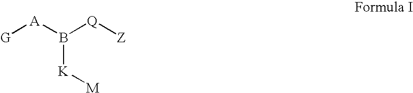
or a prodrug thereof, or a pharmaceutically acceptable salt thereof, wherein
A is SO₂ or CO;
G is Ar, Ar¹-V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′
—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

42. The method of claim 41 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.

43. A method of repairing damage caused by metastatic bone disease, the method comprising administering to a patient in need thereof a therapeutically effective amount of an EP₂ selective receptor agonist.

44. The method of claim 43 wherein the EP₂ selective receptor agonist is a compound of Formula I 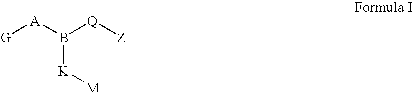
or a prodrug thereof, or a pharmaceutically acceptable salt thereof, wherein
A is SO₂ or CO;
G is Ar, Ar¹ —V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
Bis N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen., sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully, saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′- or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

45. The method of claim 44 wherein the EP₂ selective receptor agonist is (3-(((4-tert-butyl-benzyl)-(pyridine-3-sulfonyl)-amino)-methyl)-phenoxy)-acetic acid or a pharmaceutically acceptable salt thereof.
